(19) United States
(12) Patent Application Publication
(10) Pub. No.: US 2025/0186450 A1
(43) Pub. Date: Jun. 12, 2025

(54) COMBINATION THERAPY COMPRISING DGK INHIBITORS and PD-1/PD-L1 INHIBITORS

(71) Applicant: Incyte Corporation, Wilmington, DE (US)

(72) Inventors: Xiaodi Ren, Wilmington, DE (US); Rodrigo Hess, Swarthmore, PA (US); Joshua Hummel, Hockessin, DE (US); Jacob J. Lacharity, Chesterbrook, PA (US); Xiaolei Li, New Castle, DE (US); Sharada Manns, Wilmington, DE (US); Ding-Quan Qian, Newark, DE (US); Xiaozhao Wang, Moorestown, NJ (US); Bo Wei, Claymont, DE (US)

(21) Appl. No.: 18/969,874

(22) Filed: Dec. 5, 2024

Related U.S. Application Data
(60) Provisional application No. 63/610,797, filed on Dec. 15, 2023.
(60) Provisional application No. 63/607,013, filed on Dec. 6, 2023.

(51) Int. Cl. A61K 31/52; A61K 31/496; A61K 39/00; A61P 35/00; C07K 16/28

(52) U.S. Cl. CPC A61K 31/52; A61K 31/496; A61P 35/00; C07K 16/2818; A61K 2039/505

(57) ABSTRACT

The present application provides methods of treating cancer using a combination of a DGK inhibitor and an inhibitor of PD-1 and/or PD-L1.

### SEQUENCE LISTING

This application contains a Sequence Listing that has been submitted electronically as an XML file named “20443-0844001_SL_ST26.XML.” The XML file, created on Dec. 3, 2024, is 16,098 bytes in size. The material in the XML file is hereby incorporated by reference in its entirety.

### TECHNICAL FIELD

Disclosed herein are combination therapies comprising a DGK inhibitor and an inhibitor of PD-1/PD-L1, and methods of using the same to treat disorders such as cancer.

### BACKGROUND

Some cancer patients have poor long-term prognosis and/or are resistant to one or more types of treatment commonly used in the art. Therefore, a need remains for effective therapies for cancer with increased efficacy and improved safety profiles in this difficult-to-treat patient population.

### SUMMARY

The present application provides, inter alia, a method of treating a cancer in a subject, comprising administering to the subject:
(i) a diacylglycerol kinase (DGK) inhibitor; and
(ii) an inhibitor of PD-1/PD-L1.

The present invention further provides a diacylglycerol kinase (DGK) inhibitor and an inhibitor of PD-1/PD-L1 for use in any of the methods described herein.

The present invention further provides use of a diacylglycerol kinase (DGK) inhibitor and an inhibitor of PD-1/PD-L1 for the preparation of a medicament for use in any of the methods described herein.

The present application further provides a method of treating comprising administering to the subject a compound of Formula I.

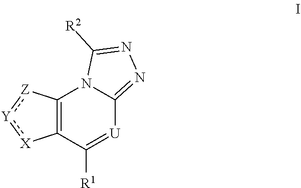

or a pharmaceutically acceptable salt thereof, wherein constituent members are defined herein, and wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H).

The present invention further provides a compound of Formula I, or a pharmaceutically acceptable salt thereof, for use a method of treating cancer in a subject, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H).

The present invention further provides use of a compound of Formula I, or a pharmaceutically acceptable salt thereof, for the preparation of a medicament for use in treating cancer in a subject, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H).

### DESCRIPTION OF DRAWINGS

FIG. 1 shows a graph depicting the tumor volume of CT26 tumor bearing mice administered (i) vehicle; (ii) 250 μg/mouse of RMP1-14 (“ANTI-PD-1”); (iii) 10 mg/kg of Compound 1; (iv) 3 mg/kg of Compound 1; (v) 10 mg/kg of Compound 1 and 250 μg/mouse of RMP1-14; or (vi) 3 mg/kg of Compound 1 and 250 μg/mouse of RMP1-14. Kruskal-Wallis Multiple Comparison Test shows statistical significance between group (v) and group (ii) (p=0.0024).

FIG. 2 shows a graph depicting the tumor volume of CT26 tumor bearing mice administered (i) vehicle; (ii) 250 μg/mouse of RMP1-14 (“ANTI-PD-1”); (iii) 10 mg/kg of Compound 1 QD; (iv) 10 mg/kg of Compound 1 Q2D; (v) 3 mg/kg of Compound 1 QD; (vi) 3 mg/kg of Compound 1 Q2D; (vii) 10 mg/kg of Compound 1 QD and 250 μg/mouse of RMP1-14; (viii) 10 mg/kg of Compound 1 Q2D and 250 μg/mouse of RMP1-14; (ix) 3 mg/kg of Compound 1 QD and 250 μg/mouse of RMP1-14; or (x) 3 mg/kg of Compound 1 Q2D and 250 μg/mouse of RMP1-14. Kruskal-Wallis Multiple Comparison Test shows statistical significance between all groups tested against group (ii) (*p<0.05; ***p<0.001; ****p<0.0001).

FIG. 3 shows a graph depicting the tumor volume of CT26 tumor bearing mice administered (i) vehicle; (ii) 250 μg/mouse of RMP1-14 (“ANTI-PD-1”); (iii) 10 mg/kg of Compound 1; (iv) 1 mg/kg of Compound 1; (v) 10 mg/kg of Compound 1 and 250 μg/mouse of RMP1-14; or (vi) 1 mg/kg of Compound 1 and 250 μg/mouse of RMP1-14. Kruskal-Wallis Multiple Comparison Test shows statistical significance between groups (v) and group (ii) (**p=0.0086), as well us between group (vi) and group (ii) (*p=0.026).

FIG. 4 shows a graph depicting the tumor volume of CT26 Clone299 tumor bearing mice administered (i) vehicle; (ii) 25 mg/kg of Compound A; (iii) 10 mg/kg of Compound 1; (iv) 3 mg/kg of Compound 1; (v) 10 mg/kg of Compound 1 and 25 mg/kg of Compound A; or (vi) 3 mg/kg of Compound 1 and 25 mg/kg of Compound A. Kruskal-Wallis Multiple Comparison Test shows statistical significance between groups (v) and group (ii) (**p=0.0068), as well us between group (v) and group (iii) (*p=0.0151).

FIGS. 5A-5B show analysis of production of IFNγ in freshly purified human CD3+ T cells co-cultured with allogenic dendritic cells differentiated from monocytes and treated with 4 nM Compound 2, 0.7 nM retifanlimab (FIG. 5A) or 0.7 nM pembrolizumab (FIG. 5B) or Compound 2 in combination with retifanlimab or pembrolizumab. Supernatant IFNγ was analyzed by HTRF and analyzed by One-way ANOVA. **p=0.0024, ***p=0.0001, ****p<0.0001.

FIGS. 6A-6B show results of combinational effect of Compound 2 with anti-PD-1 in an in vivo CT26 model. Mice were monitored for tumor growth and overt tolerability over the course of the experiment. One-way ANOVA multiple comparison analysis was used to determine statistical differences between treatment groups. * p=<0.05. ** p=<0.01.

FIG. 7 shows results of combinational effect of Compound 2 with anti-PD-1 in an in vivo MC38 model. The activity of Compound 2 was determined in mice bearing MC38 tumors (N=8/group), with treatment starting on Day 7. Tumor growth inhibition of Compound 2 at 0.5 mg/kg+anti-PD-1 was statistically significant when compared with the vehicle control group for both QD and Q2D, as well as all 1.5 mg/kg+anti-PD-1 combination groups against the vehicle control. Significance was also observed when comparing Compound 2 1.5 mg/kg+anti-PD-1 QD (p=0.0027), Q2D (p=0.0057), and Q3D (p=0.0103) with the anti-PD-1 control group. * p=<0.05. ** p=<0.01. *** p=<0.001.

FIG. 8 shows in vivo effects of Compound 2 plus anti-PD-L1 (atezolizumab) assessed in 8 to 10 weeks old human PD-1/human PD-L1 dual knock-in mice. The activity of Compound 2 was determined in mice bearing CT26 Clone299 (N=8/group), with treatment starting on Day 12. Tumor growth inhibition of Compound 2 at 1.5 mg/kg Q2D+anti-PD-L1 was statistically significant when compared with the anti-PD-L1 control group. * p=<0.05.

FIGS. 9A-9B show in vivo effects of combining Compound 3 plus anti-PD-1 (RMP1-14) in MC38 syngeneic tumor model in 6 to 8 weeks old C57Bl/6 mice. The activity of Compound 3 was determined in mice bearing MC38 tumors (N=8/group), with treatment starting on Day 7. Tumor growth inhibition of Compound 3 was observed for all groups when compared to vehicle control group. Compound 3 at 30 mg/kg+anti-PD-1 was statistically significant when compared with the anti-PD-1 control group. * p=<0.05. ** p=<0.01. ****p=<0.0001.

### DETAILED DESCRIPTION

The present application provides, inter alia, a method of treating a cancer in a subject, comprising administering to the subject:
(i) an inhibitor of diacylglycerol kinase (DGK); and
(ii) an inhibitor of PD-1/PD-L1.

The methods described herein are useful for the treatment of diseases or disorders, such as those that would benefit from the stimulation of the immune system, such as cancer and infectious diseases, and wherein the inhibitor is administered as a single agent or in combination with an antagonist of the PD1/PD-L1 axis.

### DGK Inhibitors

The DGK inhibitors (i.e., “the inhibitors of DGK”) provided herein are useful in providing a means of preventing the growth or inducing apoptosis of cancer cells. Such compounds are also useful in treating cancer cells exhibiting alterations in diacylglycerol-regulating enzymes and effectors. It is therefore anticipated that the compounds of the disclosure are useful in treating or preventing cancer, such as solid tumors.

Diacylglycerol kinases (DGKS) are a family of enzymes that regulate many biological processes, including cellular proliferation, migration, immunity and pathogenesis of diseases such as cancer. In mammalian systems, there are ten DGK family members classified into five subtypes based on shared common domains (Sakane F. et al., Int. J. Mol. Sci., 2020. 21: p 6794-6829). The diverse and specific cellular function of individual DGK isoforms is regulated through their tissue restricted expression, localization within cells and interactions with regulatory proteins (Joshi, R. P. and Koretzky, G. A., Int. J. Mol. Sci., 2013. 14: p 6649-6673).

In T lymphocytes, DGKα and ζ are the dominant DGK isoforms expressed (Krishna, S. and Zhong, X.-P., Front Immunol., 2013. 4:178). Specifically, in response to T cell receptor (TCR) activation, phospholipase Cγ1 (PLCγ1) hydrolyzes membrane phospholipid PIP2 to produce diacylglycerol (DAG) (Krishna, S. and Zhong, X.-P., Front Immunol., 2013. 4:178; Riese, M. J. et al., Front Cell Dev Biol., 2016. 4:108). In turn, DAG functions as a second messenger to recruit RasGRP1 and PKCθ to the cell membrane and thereby initiates multiple downstream signaling events resulting in T cell activation. To prevent hyperactivation of T cells, DGKα and ζ tightly regulate the levels of intracellular DAG by phosphorylating DAG to produce phosphatidic acid (PA). Both mouse and human cell line genetic studies support the important regulatory role of DGKα and ζ in T cell activation. Knockout or depletion of DGKα and ζ has been reported to enhance T cell activation, cytokine production and proliferation. Furthermore, knockout of both DGKα and ζ show even greater T-cell activation over individual knockouts, indicating a non-redundant role of these two isoforms (Riese, M. J. et al., Cancer Res., 2013. 73:p 3566-3577; Jung, I.-Y. et al., Cancer Res., 2018. 78: p 4692-4703). Thus, DGKα and ζ, by regulating cellular DAG levels link lipid metabolism and intracellular signaling cascades and function as key regulators of T cell activation.

Cytotoxic T lymphocytes (CTLs) are a major component of the adaptive immune system that recognize and kill cells with bacterial or viral infections, or cells displaying abnormal proteins, such as tumor antigens. However, cancer cells can evolve to utilize multiple mechanisms that mimic peripheral immune tolerance to avoid immune surveillance and killing by CTLs. Such mechanisms include downregulation of antigen presentation, suppression of T cell function through increased expression of inhibitory molecules, as well as increased production of immunosuppressive proteins in the tumor microenvironment (Speiser, D. E. et al., Nat. Rev. Immunol., 2016. 16: p. 599-611, Gonzalez H. et al., Genes & Dev., 2018. 32:p 1267-1284). Immune checkpoint therapy (ICT) by blocking inhibitory molecules such as PD(L)-1 and CTLA4, can restore T cell activity and have been clinically useful in treating many different types of cancers. However, only subsets of patients respond to ICT due to primary or acquired resistance (Sharma, P. et al., Cell. 2017. 168: p 707-723). Thus, despite the significant recent clinical successes of immunotherapies to treat cancer, resistance remains a challenge (Sharma, P., et al., Cancer Discov., 2021. 11: p 838-857).

Overexpression of DGKα and ζ has been observed in tumor infiltrating lymphocytes (TILs) from human tumors and proposed to suppress T cell function. Importantly, significant immune-mediated antitumor activity has been shown in DGKα and DGKζ deficient mouse models (Merida, I. et al., Adv. Biol. Regul., 2017. 63:p 22-31, Prinz, P. U. et al., J. Immunol., 2012. 188:p 5990-6000). Furthermore, DGKα and DGKζ deficient T cells are resistant to several immunosuppressive factors within the tumor microenvironment such as TGFβ, PGE2 and adenosine, and to other T cell inhibitory pathways such as PD(L)-1 mediated immune suppression (Riese, M. J. et al., Cancer Res., 2013. 73:p 3566-77; Jung, I.-Y. et al. (2018) Cancer Res., 2018. 78:p 4692-4703; Arranz-Nicolas, J. et al., Cancer Immunol. Immunother., 2018. 67:p 965-980; Riese, M. J. et al., Front. Cell Dev. Biol., 2016. 4:108). Thus DGKα and DGKζ are attractive targets as immunotherapies alone or in combination with current ICT therapies such as PD(L)-1 and CTLA4. By targeting T cell lipid metabolism, DGKα and DGKζ inhibition can potentially restore antitumor immunity in subsets of patient who have primary or acquired immune resistance and are consequently refractory to current ICTs. In addition to its function in T lymphocytes, DGKα and DGKζ, by regulating DAG level in cancer cells, have also been reported to directly contribute to cancer proliferation, migration, invasion and survival. Thus, DGK inhibition may have direct antitumor effect by interfering with tumor intrinsic oncogenic survival pathways (Cooke, M. and Kaznietz, M. G., Sci. Signal., 2022. 15:eabo0264).

The DGK inhibitors described herein can be selective. By “selective,” it is meant that the DGK inhibitor binds to or inhibits DGKα or DGKζ with greater affinity or potency, respectively, compared to at least one other DGK isoforms, or kinase, etc. In some embodiments, selectivity can be at least about 2-fold, 5-fold, 10-fold, at least about 20-fold, at least about 50-fold, at least about 100-fold, at least about 200-fold, at least about 500-fold or at least about 1000-fold. The DGK inhibitors of the present disclosure can also be dual antagonists (i.e., inhibitors), e.g. inhibit both DGKα and DGKζ kinases. In some embodiments, the DGK inhibitors of the invention are selective inhibitors of DGKα (e.g., over one or more other DGK isoforms, or kinase, etc.). In some embodiments, the DGK inhibitors of the invention are selective inhibitors of DGKζ (e.g., over one or more other DGK isoforms, or kinase, etc.). Selectivity can be measured by methods routine in the art. In some embodiments, selectivity can be tested at the Kₘ ATP concentration of each enzyme. In some embodiments, the selectivity of DGK inhibitors of the invention can be determined by cellular assays associated with particular DGK kinase activity.

Based on compelling evidence that DGKα and DGKζ negatively regulate signaling pathways downstream of the T cell receptor, developing DGK inhibitors can boost T cell effector function and inhibit tumor progression. DGK inhibitors can be used to treat, alone or in combination with other therapies, cancers including solid tumors and hematological malignancies, including renal cell carcinoma, mesothelioma, glioblastoma multiforme, colorectal cancer, melanoma, pancreatic cancer (Chen, S. S. et al., Front. Cell Dev. Biol., 2016. 4:130; Gu, J. et al., Oncoimmunol., 2021. 10, e1941566; Jung I.-Y. et al., Cancer Res., 2018. 78:p 4692-4703; Sitaram, P., et al., Int. JMol. Sci., 2019. 20:p 5821-5848; Wesley, E. M., et al., Immunohorizons, 2018. 2:p 107-118). Furthermore, pharmacological inhibition of DGK provides benefit to control viral infections, and can be used to treatment such viral infections including Coronavirus infection, HIV infection, hepatitis virus infection in preclinical model (Harabuchi, S. et al., Front. Immunol., 2022. 13:1032113).

In addition, DGKα has been shown to enhance esophageal squamous cell carcinoma (ESCC), and human hepatocellular carcinoma (HCC) progression (Chen, J. et al., Oncogene, 2019. 38: p 2533-2550; Takeishi, K. et al., J. Hepatol., 2012. 57:p 77-83), to support colon and breast cancer growth in three-dimensional (3D) culture (Torres-Ayuso, P. et al., Oncotarget, 2014. 5:p 9710-9726), to enhance mammary carcinoma invasiveness (Rainero, E. et al., PLOS ONE, 2014. 9(6): e97144) and promote metastasis of non-small cell lung cancer (NSCLC) (Fu, L. et al., Cancer letters, 2022. 532: 215585) whereas DGKζ has been implicated as a potential oncogene in osteosarcoma proliferation (Yu, W. et al., Front. Oncol., 2019. 8:655) and contributed to enhanced invasion of human metastatic colon cancer cells (Cai, K. et al., BMC Cancer, 2014. 14:208). It has also been reported DGK inhibition has the potential to reduce immunopathology in X-linked lymphoproliferative disease patient (Velnati, S. et al., Eur. J. Med. Chem., 2019. 164: p 378-390; Ruffo, E. et al., Sci. Transl. Med. 2016. 8 (321):321ra7). DGK inhibitors of the present application may have selective activities towards one or both DGKα and DGKζ. These DGK inhibitors alone or in combination with PD-1/PD-L1 inhibitors described herein can be used in treatment of cancer.

In some embodiments, the DGK inhibitor provided herein is a compound of

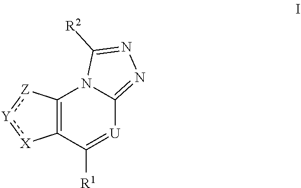

or a pharmaceutically acceptable salt thereof, wherein:
each [symbol] is a single or double bond, wherein at least one [symbol] is a double bond;
U is CH or N; [symbol]
X is CR⁴, N, NR⁴, S, or O;
Y is CR⁵ or N;
Z is CR⁶, NR⁶, or S;
R¹ is Cy¹ or L-Cy¹;
L is NR^{c7}, O, C₁₋₃ alkyl, C₂₋₃ alkenyl, or C₂₋₃ alkynyl;
Cy¹ is a C₃₋₁₀ cycloalkyl, 5-15 membered heteroaryl, or 4-15 membered heterocycloalkyl, wherein the C₃₋₁₀ cycloalkyl, 5-15 membered heteroaryl or 4-15 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, or 4 independently selected R¹ substituents;
each R^{1B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, and OR^{a12}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1B} are each optionally substituted with 1, 2, 3, or 4 independently selected R^{1C} substituents;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, and OR^{a13};
each R^{a13} is independently selected from H, C₁₋₆ alkyl, and C₁₋₆ haloalkyl;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, halo, C₁₋₆ alkyl, C₁₋₆ haloalkyl, C₂₋₆ alkenyl, and C₂₋₆ alkynyl;
R⁵ is selected from H, D, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, C₂₋₆ alkynyl, C₃₋₁₀ cycloalkyl-C₁₋₆ alkyl-, or (4-10 membered heterocycloalkyl)-C₁₋₆ alkyl-, wherein the C₁₋₆ alkyl, C₁₋₆ haloalkyl, C₂₋₆ alkenyl, C₂₋₆ alkynyl, C₃₋₁₀ cycloalkyl-C₁₋₆ alkyl-, and (4-10 membered heterocycloalkyl)-C₁₋₆ alkyl- are each optionally substituted by 1, 2, 3, or 4 independently selected R^{6A} substitutents;
each R^{6A} is independently selected from halo, C₁₋₆ alkyl, C₁₋₆ haloalkyl, C₂₋₆ alkenyl, C₂₋₆ alkynyl, CN, and NR^{c61}R^{d61};
each R^{c61} and R^{d61} is independently selected from H and C₁₋₆ alkyl; and
R^{c7} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments of Formula I.
each [symbol] is a single or double bond, wherein at least one [symbol] is a double bond;
U is CH or N;
X is CR⁴, N, NR⁴, S, or O;
Y is CR⁵ or N;
Z is CR⁶, NR⁶, or S;
R¹ is Cy¹ or L-Cy¹;
L is NR^{c7}, O, C₁₋₃ alkyl, C₂₋₃ alkenyl, or C₂₋₃ alkynyl;
Cy¹ is a C₃₋₁₀ cycloalkyl, 5-15 membered heteroaryl, or 4-15 membered heterocycloalkyl, wherein the C₃₋₁₀ cycloalkyl, 5-15 membered heteroaryl or 4-15 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, or 4 independently selected R¹ substituents;
each R^{1B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, and OR^{a12}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of RB are each optionally substituted with 1, 2, 3, or 4 independently selected R^{1C} substituents;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, and CN;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, halo, C₁₋₆ alkyl, C₁₋₆ haloalkyl, C₂₋₆ alkenyl, and C₂₋₆ alkynyl;
R⁵ is selected from H, D, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, and C₂₋₆ alkynyl; and
R^{c7} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments of the previous embodiment, R⁵ is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents.

In some embodiments of Formula I:
U is CH or N;
X is CR⁴, N, NR⁴, S, or O;
Y is CR⁵ or N;
Z is CR⁶, NR⁶, or S;
R¹ is Cy¹;
Cy¹ is a C₃₋₇ cycloalkyl, 5-6 membered heteroaryl, or 4-7 membered heterocycloalkyl, wherein the C₃₋₇ cycloalkyl, 5-6 membered heteroaryl, and 4-7 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{1A} are each optionally substituted with 1, 2, 3, or 4 independently selected R¹ substituents;
each R^{1B} is independently selected from C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, and OR^{a12}, wherein the C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of RB are each optionally substituted with 1, 2, 3, or 4 independently selected R^{1C} substituents;
each R^{1C} is independently selected from halo, C₁₋₆ alkyl, C₁₋₆ haloalkyl, C₂₋₆ alkenyl, C₂₋₆ alkynyl, CN, and OR^{a13};
each R^{a12} is independently selected from H, C₁₋₆ alkyl, C₁₋₆ haloalkyl, C₂₋₆ alkenyl, and C₂₋₆ alkynyl;
each R^{a13} is independently selected from H, C₁₋₆ alkyl, and C₁₋₆ haloalkyl;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents;
R⁴ is H or C₁₋₆ alkyl;
R⁵ is selected from H, D, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, C₂₋₆ alkynyl, C₃₋₇ cycloalkyl-C₁₋₆ alkyl-, or (4-7 membered heterocycloalkyl)-C₁₋₆ alkyl-, wherein the C₁₋₆ alkyl, C₃₋₇ cycloalkyl-C₁₋₆ alkyl-, and (4-7 membered heterocycloalkyl)-C₁₋₆ alkyl- are each optionally substituted by NR^{c61}R^{d61}; and
each R^{c61} and R^{d61} is independently selected from H and C₁₋₆ alkyl.

In some embodiments of the previous embodiment, R⁵ is selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R⁵ are each optionally substituted with 1, 2, 3, or 4 independently selected R^{5A} substituents.

In some embodiments of Formula I:
each [symbol] is a single or double bond, wherein at least one [symbol] is a double bond;
U is CH or N;
X is CR⁴, N, NR⁴, S, or O;
Y is CR⁵ or N;
Z is CR⁶, NR⁶, or S;
R¹ is Cy¹ or L-Cy¹;
L is NR^{c7}, O, C₁₋₃ alkyl, C₂₋₃ alkenyl, or C₂₋₃ alkynyl;
Cy¹ is a C₃₋₁₀ cycloalkyl, 5-10 membered heteroaryl, or 4-10 membered heterocycloalkyl, wherein the C₃₋₁₀ cycloalkyl, 5-10 membered heteroaryl, or 4-10 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, or 4 independently selected R¹ substituents;
each R^{1B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, and OR^{a12}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of RB are each optionally substituted with 1, 2, 3, or 4 independently selected R^{1C} substituents;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, and CN;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, halo, C₁₋₆ alkyl, C₁₋₆ haloalkyl, C₂₋₆ alkenyl, and C₂₋₆ alkynyl;
R⁵ is selected from H, D, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, and C₂₋₆ alkynyl; and
R^{c7} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments of the previous embodiment, R⁵ is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents.

In some embodiments of Formula I:
U is CH or N;
X is CH, CCH₃, N, —NCH₂CH₃, S, or O;
Y is CR⁵ or N;
Z is CH, NCH₃, NCH₂CH₂N(CH₃)₂, NCH₂-cyclopropyl, NCH₂-tetrahydrofuranyl, or S;
R¹ is Cy¹;
Cy¹ is a 4-7 membered heterocycloalkyl which is optionally substituted with 1, 2, 3, or 4 independently selected R^{1A} substituents;
each R^{1A} is independently selected from C₁₋₆ alkyl, each of which are optionally substituted with 1, 2, 3, or 4 independently selected R^{1B} substituents;
each R^{1B} is independently selected from C₁₋₆ alkyl, phenyl, C₃₋₇ cycloalkyl, 5-6 membered heteroaryl, 4-7 membered heterocycloalkyl, CN, and OR^{a12}, wherein the C₁₋₆ alkyl, phenyl, C₃₋₇ cycloalkyl, 5-6 membered heteroaryl, and 4-7 membered heterocycloalkyl of R^{1B} are each optionally substituted with 1, 2, 3, or 4 independently selected R^{1C} substituents;
each R^{1C} is independently selected from halo, C₁₋₆ alkyl, C₁₋₆ haloalkyl, C₂₋₆ alkenyl, C₂₋₆ alkynyl, CN, and OR^{a13};
each R^{a12} is independently selected from H, C₁₋₆ alkyl, C₁₋₆ haloalkyl, C₂₋₆ alkenyl, and C₂₋₆ alkynyl;
each R^{a13} is independently H, C₁₋₆ alkyl, or C₁₋₆ haloalkyl;
R⁵ is selected from H, D, C₁₋₆ alkyl, C₁₋₆ haloalkyl, and 5-6 membered heteroaryl, wherein the C₁₋₆ alkyl and 5-6 membered heteroaryl of R⁵ are each optionally substituted with 1, 2, 3, or 4 independently selected R^{5A} substituents; and
each R^{SA} is independently selected from halo, C₁₋₆ alkyl, C₁₋₆ haloalkyl, C₂₋₆ alkenyl, C₂₋₆ alkynyl, and CN.

In some embodiments of Formula I:
U is CH or N;
X is CH, CCH₃, N, —NCH₂CH₃, S, or O;
Y is CR⁵ or N;
Z is CH, NCH₃, NCH₂CH₂N(CH₃)₂, NCH₂-cyclopropyl, NCH₂-tetrahydrofuranyl, or S;
R¹ is Cy¹;
Cy¹ is a 4-7 membered heterocycloalkyl which is optionally substituted with 1, 2, 3, or 4 independently selected R^{1A} substituents;
each R^{1A} is independently selected from C₁₋₆ alkyl, each of which are optionally substituted with 1, 2, 3, or 4 independently selected R^{1B} substituents;
each R^{1B} is independently selected from C₁₋₆ alkyl, phenyl, C₃₋₇ cycloalkyl, 5-6 membered heteroaryl, 4-7 membered heterocycloalkyl, CN, and OR^{a12}, wherein the phenyl, C₃₋₇ cycloalkyl, 5-6 membered heteroaryl, and 4-7 membered heterocycloalkyl of R^{1B} are each optionally substituted with 1, 2, 3, or 4 independently selected R^{1C} substituents;
each R^{1C} is independently selected from halo, C₁₋₆ alkyl, C₁₋₆ haloalkyl, C₂₋₆ alkenyl, C₂₋₆ alkynyl, CN, and OR^{a13};
each R^{a12} is independently selected from H, C₁₋₆ alkyl, C₁₋₆ haloalkyl, C₂₋₆ alkenyl, and C₂₋₆ alkynyl;
each R^{a13} is independently H, C₁₋₆ alkyl, or C₁₋₆ haloalkyl;
R⁵ is selected from H, C₁₋₆ alkyl, C₁₋₆ haloalkyl, and 5-6 membered heteroaryl, wherein the C₁₋₆ alkyl and 5-6 membered heteroaryl of R⁵ are each optionally substituted with 1, 2, 3, or 4 independently selected R^{5A} substituents; and
each R^{5A} is independently selected from halo, C₁₋₆ alkyl, C₁₋₆ haloalkyl, C₂₋₆ alkenyl, C₂₋₆ alkynyl, and CN.

In some embodiments of Formula I:
U is CH or N;
X is CR⁴, N, NR⁴, S, or O;
Y is CR⁵ or N;
Z is CR⁶, NR⁶, or S;
R¹ is Cy¹;
Cy¹ is a C₃₋₇ cycloalkyl, 5-6 membered heteroaryl, or 4-7 membered heterocycloalkyl, wherein the C₃₋₇ cycloalkyl, 5-6 membered heteroaryl, and 4-7 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{1A} are each optionally substituted with 1, 2, 3, or 4 independently selected R¹ substituents;
each R^{1B} is independently selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, and OR^{a12}, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1B} are each optionally substituted with 1, 2, 3, or 4 independently selected R^{1C} substituents;
each R^{1C} is independently selected from halo, C₁₋₆ alkyl, C₁₋₆ haloalkyl, C₂₋₆ alkenyl, C₂₋₆ alkynyl, and CN;
each R^{a12} is independently selected from H, C₁₋₆ alkyl, C₁₋₆ haloalkyl, C₂₋₆ alkenyl, and C₂₋₆ alkynyl;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents;
R⁴ is H or C₁₋₆ alkyl;
R⁵ is selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, and 4-7 membered heterocycloalkyl, wherein the C₁₋₆ alkyl, C₂₋₆ alkenyl, C₂₋₆ alkynyl, phenyl, C₃₋₇ cycloalkyl, 5-6 membered heteroaryl, and 4-7 membered heterocycloalkyl of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, and C₂₋₆ alkynyl.

In some embodiments of Formula I:
U is CH or N;
X is CH, CCH₃, N, —NCH₂CH₃, S, or O;
Y is CR⁵ or N;
Z is CH, NCH₃, or S;
R¹ is Cy¹;
Cy¹ is a 4-7 membered heterocycloalkyl which is optionally substituted with 1, 2, 3, or 4 independently selected R^{1A} substituents;
each R^{1A} is independently selected from C₁₋₆ alkyl, each of which are optionally substituted with 1, 2, 3, or 4 independently selected R^{1B} substituents;
each R^{1B} is independently selected from phenyl, C₃₋₇ cycloalkyl, 5-6 membered heteroaryl, 4-7 membered heterocycloalkyl, CN, and OR^{a12}, wherein the phenyl, C₃₋₇ cycloalkyl, 5-6 membered heteroaryl, and 4-7 membered heterocycloalkyl of R^{1B} are each optionally substituted with 1, 2, 3, or 4 independently selected R^{1C} substituents;
each R^{1C} is independently selected from halo, C₁₋₆ alkyl, C₁₋₆ haloalkyl, C₂₋₆ alkenyl, C₂₋₆ alkynyl, and CN;
each R^{a12} is independently selected from H, C₁₋₆ alkyl, C₁₋₆ haloalkyl, C₂₋₆ alkenyl, and C₂₋₆ alkynyl;
R⁵ is selected from H, C₁₋₆ alkyl, C₁₋₆ haloalkyl, and 5-6 membered heteroaryl, wherein the C₁₋₆ alkyl and 5-6 membered heteroaryl of R⁵ are each optionally substituted with 1, 2, 3, or 4 independently selected R^{5A} substituents; and
each R^{5A} is independently selected from halo, C₁₋₆ alkyl, C₁₋₆ haloalkyl, C₂₋₆ alkenyl, C₂₋₆ alkynyl, and CN.

In some embodiments, the compound of Formula I is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof.

In some embodiments, the compound of Formula I is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof.

In some embodiments, the compound of Formula I is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula I is 4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula I is 4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula I is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the DGK inhibitor provided herein is a compound of

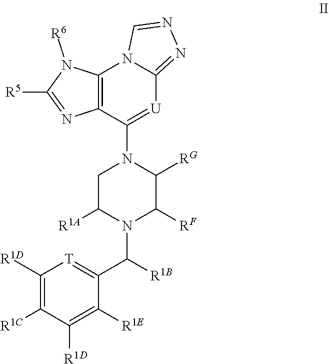

or a pharmaceutically acceptable salt thereof, wherein:
U is CH or N;
T is CH, C—R^{1E}, or N;
R^{1A} is C₁₋₃ alkyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from halo, C₁₋₃ haloalkyl, and C₁₋₃ alkoxy;
each R^{1D} is independently H, fluoro, chloro, or trifluoromethyl;
or R^{1C} and R^{1D}, together with the atoms of the phenyl or piperazyl ring form a 10 membered bicyclic heteroaryl, which is substituted with C₁₋₃ haloalkyl;
each R^{1E} independently is H or fluoro;
R^{1F} is H; or
R^{1A} and R^{1F}, together with the atoms of the piperazyl ring, form an 8 membered bridged heterocycloalkyl;
R^{1G} is H or methyl;
R⁵ is H or C₁₋₃ alkyl; and
R⁶ is C₃₋₇ cycloalkyl-C₁₋₃ alkyl-, wherein the C₃₋₇ cycloalkyl-C₁₋₃ alkyl- is optionally substituted with 1 or 2 OH substituents; or
R⁶ is (4-7 membered heterocycloalkyl)-C₁₋₃ alkyl-; or
R⁶ is di(C₁₋₆ alkyl)amino-C₁₋₃ alkyl-.

In some embodiments, the DGK inhibitor provided herein is a compound of Formula II:

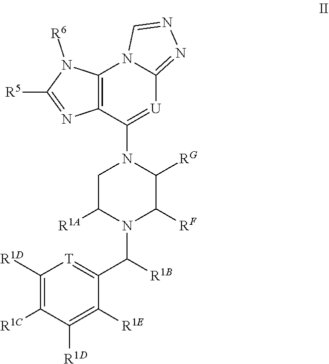

or a pharmaceutically acceptable salt thereof, wherein:
U is CH or N;
T is CH, C—R^{1E}, or N;
R^{1A} is C₁₋₃ alkyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from halo, C₁₋₃ haloalkyl, and C₁₋₃ alkoxy;
each R^{1D} is independently H, fluoro, chloro, or trifluoromethyl;
or R^{1C} and R^{1D}, together with the atoms of the phenyl or piperazyl ring form a 10 membered bicyclic heteroaryl, which is substituted with C₁₋₃ haloalkyl;
each R^{1E} independently is H or fluoro;
R^{1F} is H; or
R^{1A} and R^{1F}, together with the atoms of the piperazyl ring, form an 8 membered bridged heterocycloalkyl;
R^{1G} is H or methyl;
R⁵ is H or C₁₋₃ alkyl; and
R⁶ is C₃₋₇ cycloalkyl-C₁₋₃ alkyl-, wherein the C₃₋₇ cycloalkyl-C₁₋₃ alkyl- is optionally substituted with 1 or 2 OH substituents; or
R⁶ is (4-7 membered heterocycloalkyl)-C₁₋₃ alkyl-.

In some embodiments of Formula II:
U is CH and T is CH or C—R^{1E}; or
U is N and T is CH or C—R^{1E}; or
U is N and T is N;
R^{1A} is methyl or ethyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from fluoro, chloro, bromo, difluoromethyl, trifluoromethyl, and methoxy;
one R^{1D} is H and a second R^{1D} is selected from fluoro, chloro, and trifluoromethyl;
or R^{1C} and R^{1D}, together with the atoms of the phenyl or piperazyl ring form a 10 membered bicyclic heteroaryl, which is substituted with trifluoromethyl; each R^{1E} is independently H or fluoro;
R^{1F} is H;
or R^{1A} and R^{1F}, together with the atoms of the piperazyl ring, form an 8 membered bridged heterocycloalkyl;
R^{1G} is H or methyl;
R⁵ is H or methyl; and
R⁶ is selected from (dimethylamino)ethyl, (hydroxycyclopentyl)methyl, and tetrahydrofuranylmethyl.

In some embodiments of Formula II:
U is CH and T is CH or C—R^{1E}; or
U is N and T is CH or C—R^{1E}; or
U is N and T is N;
R^{1A} is methyl or ethyl;
R^{1B} is selected from H, C₁₋₆ alkyl, C₁₋₆ haloalkyl, phenyl, pyridinyl, and C₃₋₇ cycloalkyl, wherein the phenyl, pyridinyl, and C₃₋₇ cycloalkyl are each optionally substituted with 1, 2, or 3 substituents independently selected from halo and C₁₋₃ haloalkyl;
R^{1C} is selected from fluoro, chloro, bromo, difluoromethyl, trifluoromethyl, and methoxy;
one R^{1D} is H and a second R^{1D} is selected from fluoro, chloro, and trifluoromethyl;
or R^{1C} and R^{1D}, together with the atoms of the phenyl or piperazyl ring form a 10 membered bicyclic heteroaryl, which is substituted with trifluoromethyl;
each R^{1E} is independently H or fluoro;
R^{1F} is H;
or R^{1A} and R^{1F}, together with the atoms of the piperazyl ring, form an 8 membered bridged heterocycloalkyl;
R^{1G} is H or methyl;
R⁵ is H or methyl; and
R⁶ is (hydroxycyclopentyl)methyl or tetrahydrofuranylmethyl.

In some embodiments, the compound of Formula II is selected from:
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
   or a pharmaceutically acceptable salt thereof.

In some embodiments, the compound of Formula II is 4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine, or a pharmaceutically acceptable salt thereof.

In some embodiments, the compound of Formula II is 4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine, or a pharmaceutically acceptable salt thereof.

In some embodiments, the compound of Formula II is 4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine, or a pharmaceutically acceptable salt thereof.

In some embodiments, the compound of Formula II is 4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine, or a pharmaceutically acceptable salt thereof.

In some embodiments, the compound of Formula II is 4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine, or a pharmaceutically acceptable salt thereof.

In some embodiments, the compound of Formula II is 4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine, or a pharmaceutically acceptable salt thereof.

In some embodiments, the compound of Formula II is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof.

In some embodiments, the DGK inhibitor provided herein is a compound selected from Table A, or a pharmaceutically acceptable salt thereof.

TABLE A

| Name | Structure |
|---|---|
| 4-((2S,5R)-4-(bis(4-fluorophenyl)methyl)- 2,5-dimethylpiperazin-1-yl)-1-(((R)- tetrahydrofuran-2-yl)methyl)-1H- [1,2,3]triazolo[4,5-e][1,2,4]triazolo[4,3- a]pyrimidine (“Compound 1”) | 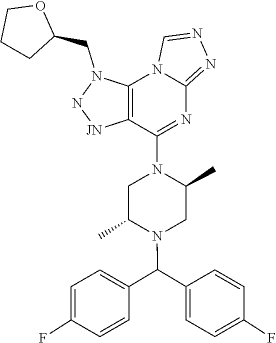 |
| 4-((2S,5R)-4-((3,3-difluorocyclobutyl)(4- (trifluoromethyl)phenyl)methyl)-2,5- dimethylpiperazin-1-yl)-2-methyl-1-(((S)- tetrahydrofuran-2-yl)methyl)-1H- [1,2,4]triazolo[3,4-b]purine | 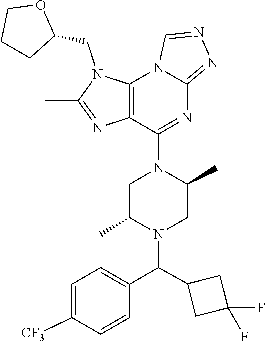 |
| 4-((2S,5R)-4-((3-chloro-4- fluorophenyl)(3,3- difluorocyclobutyl)methyl)-2,5- dimethylpiperazin-1-yl)-2-methyl-1-(((S)- tetrahydrofuran-2-yl)methyl)-1H- [1,2,4]triazolo[3,4-b]purine | 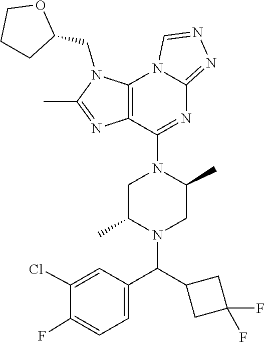 [SMILES: Cc1nc2c(N3C[C@@H](C)N(C(c4ccc(F)c(Cl)c4)C4CC(F)(F)C4)C[C@@H]3C)nc3nncn3c2n1C[C@@H]1CCCO1] |
| 4-((2S,5R)-4-((4-chloro-3- fluorophenyl)(3,3- difluorocyclobutyl)methyl)-2,5- dimethylpiperazin-1-yl)-2-methyl-1-(((S)- tetrahydrofuran-2-y1)methyl)-1H- [1,2,4]triazolo[3,4-b]purine | 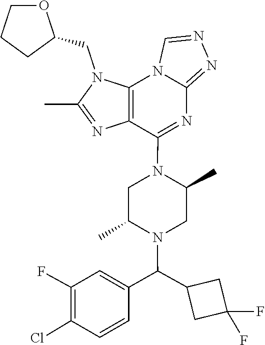 [SMILES: Cc1nc2c(N3C[C@@H](C)N(C(c4ccc(Cl)c(F)c4)C4CC(F)(F)C4)C[C@@H]3C)nc3nncn3c2n1C[C@@H]1CCCO1] |
| 4-((2S,5R)-4-(bis(4-chlorophenyl)methyl)- 2,5-dimethylpiperazin-1-yl)-2-methyl-1- (((S)-tetrahydrofuran-2-yl)methyl)-1H- [1,2,4]triazolo[3,4-b]purine | 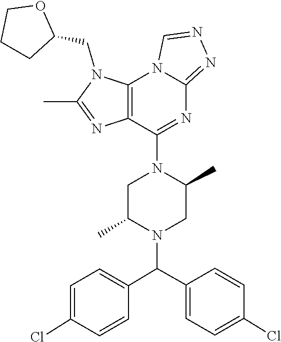 [SMILES: Cc1nc2c(N3C[C@@H](C)N(C(c4ccc(Cl)cc4)c4ccc(Cl)cc4)C[C@@H]3C)nc3nncn3c2n1C[C@@H]1CCCO1] |
| 4-((2S,5R)-4-((3,3-difluorocyclobutyl)(4- (trifluoromethyl)phenyl)methyl)-2,5- dimethylpiperazin-1-yl)-1-(((S)- tetrahydrofuran-2-y1)methyl)-1H- [1,2,4]triazolo[3,4-b]purine | 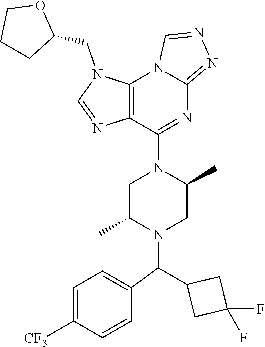 |
| 4-((2S,5R)-4-((4-chlorophenyl)((S)-2,2- difluorocyclopropyl)methyl)-2,5- dimethylpiperazin-1-yl)-1-(((S)- tetrahydrofuran-2-y1)methyl)-1H- [1,2,4]triazolo[3,4-b]purine | 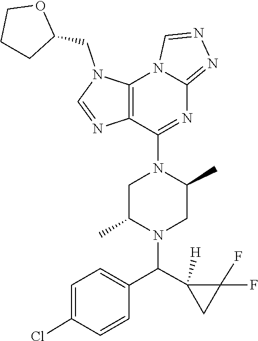 |
| 4-((2S,5R)-4-((4-chlorophenyl)((R)-2,2- difluorocyclopropyl)methyl)-2,5- dimethylpiperazin-1-yl)-1-(((S)- tetrahydrofuran-2-yl)methyl)-1H- [1,2,4]triazolo[3,4-b]purine | 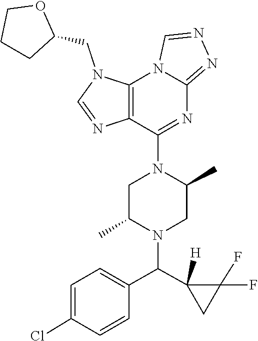 [SMILES: C[C@@H]1CN(c2nc3nncn3c3c2ncn3C[C@@H]2CCCO2)[C@@H](C)CN1C(c1ccc(Cl)cc1)[C@H]1CC1(F)F] |
| 4-((2S,5R)-4-((4-chlorophenyl)((S)-2,2- difluorocyclopropyl)methyl)-2,5- dimethylpiperazin-1-yl)-2-methyl-1-(((S)- tetrahydrofuran-2-yl)methyl)-1H- [1,2,4]triazolo[3,4-b]purine | 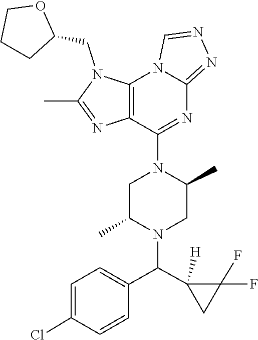 [SMILES: Cc1nc2c(N3C[C@@H](C)N(C(c4ccc(Cl)cc4)[C@@H]4CC4(F)F)C[C@@H]3C)nc3nncn3c2n1C[C@@H]1CCCO1] |
| 4-((2S,5R)-4-(1-(4-chlorophenyl)-3- methylbutyl)-2,5-dimethylpiperazin-1-yl)- 1-(((S)-tetrahydrofuran-2-yl)methyl)-1H- [1,2,4]triazolo[3,4-b]purine | 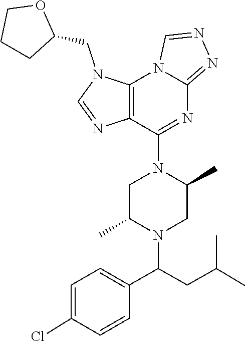 |
| 4-((2S,5R)-4-(1-(4-chloro-3-fluorophenyl)- 3-methylbutyl)-2,5-dimethylpiperazin-1- yl)-2-methyl-1-(((S)-tetrahydrofuran-2- yl)methyl)-1H-[1,2,4]triazolo[3,4-b]purine | 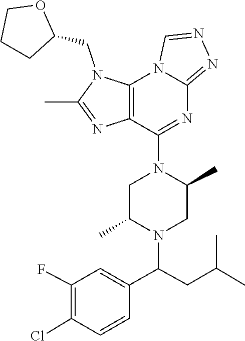 |
| 4-((2S,5R)-4-((4-chlorophenyl)(3,3- difluorocyclobutyl)methyl)-2,5- dimethylpiperazin-1-yl)-1-(((S)- tetrahydrofuran-2-yl)methyl)-1H- [1,2,4]triazolo[3,4-b]purine | 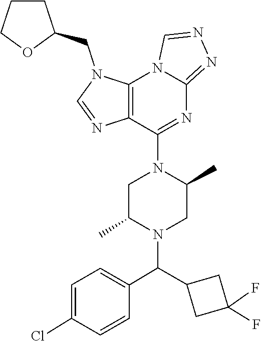 |
| 4-((2S,5R)-4-((4-chlorophenyl)(3,3- difluorocyclobutyl)methyl)-5-ethyl-2- methylpiperazin-1-yl)-1-(((S)- tetrahydrofuran-2-yl)methyl)-1H- [1,2,4]triazolo[3,4-b]purine | 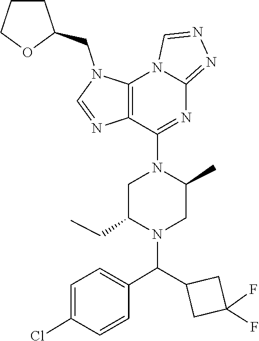 [SMILES: CC[C@@H]1CN(c2nc3nncn3c3c2ncn3C[C@@H]2CCCO2)[C@@H](C)CN1C(c1ccc(Cl)cc1)C1CC(F)(F)C1] |
| 4-((2S,5R)-4-(2-fluoro-4- (trifluoromethyl)benzyl)-2,5- dimethylpiperazin-1-yl)-2-methyl-1-(((S)- tetrahydrofuran-2-yl)methyl)-1H- [1,2,4]triazolo[3,4-b]purine | 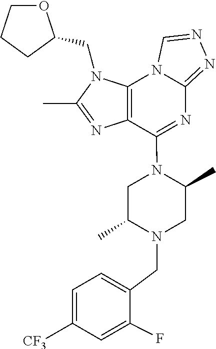 |
| 4-((2S,5R)-5-ethyl-2-methyl-4-((S)-1-(4- (trifluoromethyl)phenyl)ethyl)piperazin-1- yl)-2-methyl-1-(((S)-tetrahydrofuran-2- yl)methyl)-1H-[1,2,4]triazolo[3,4-b]purine | 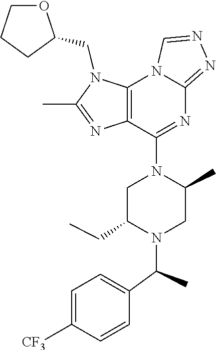 |
| 4-((2S,5R)-5-ethyl-2-methyl-4-((R)-1-(4- (trifluoromethyl)phenyl)ethyl)piperazin-1- yl)-2-methyl-1-(((S)-tetrahydrofuran-2- yl)methyl)-1H-[1,2,4]triazolo[3,4-b]purine | 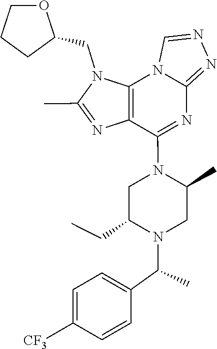 |
| 4-((2S,5R)-4-((3,3-difluorocyclobutyl)(4- (trifluoromethyl)phenyl)methyl)-2,5- dimethylpiperazin-1-yl)-2-methyl-1-(((S)- tetrahydrofuran-2-yl)methyl)-1H- imidazo[4,5-e][1,2,4]triazolo[4,3- a]pyridine | 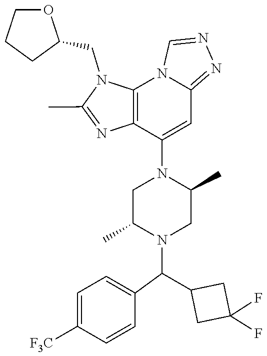 |
| 4-((2S,5R)-4-((4-chlorophenyl)((S)-2,2- difluorocyclopropyl)methyl)-2,5- dimethylpiperazin-1-yl)-1-(((S)- tetrahydrofuran-2-yl)methyl)-1H- imidazo[4,5-e][1,2,4]triazolo[4,3- a]pyridine | 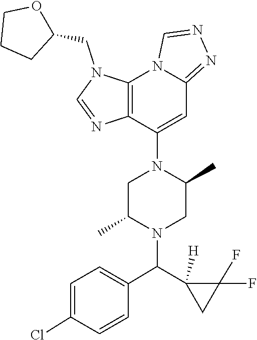 [SMILES: C[C@@H]1CN(c2cc3nncn3c3c2ncn3C[C@@H]2CCCO2)[C@@H](C)CN1C(c1ccc(Cl)cc1)[C@@H]1CC1(F)F] |
| 4-((2S,5R)-4-((4-chlorophenyl)(3,3- difluorocyclobutyl)methyl)-5-ethyl-2- methylpiperazin-1-yl)-2-methyl-1-(((S)- tetrahydrofuran-2-yl)methyl)-1H- imidazo[4,5-d][1,2,4]triazolo[4,3- a]pyridine | 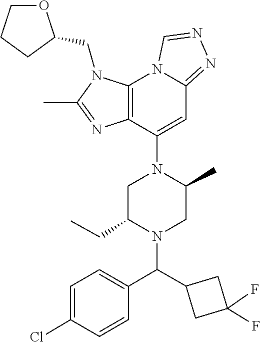 [SMILES: CC[C@@H]1CN(c2cc3nncn3c3c2nc(C)n3C[C@@H]2CCCO2)[C@@H](C)CN1C(c1ccc(Cl)cc1)C1CC(F)(F)C1] |
| 4-((2S,5R)-4-((S)-1-(4- chlorophenyl)propyl)-2,5- dimethylpiperazin-1-yl)-1-(((S)- tetrahydrofuran-2-yl)methyl)-1H- [1,2,4]triazolo[3,4-b]purine | 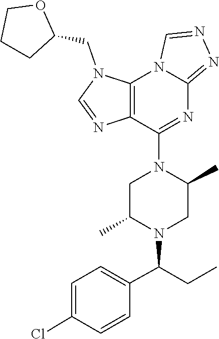 |
| 4-((2S,5R)-4-((R)-1-(4- chlorophenyl)propyl)-2,5- dimethylpiperazin-1-yl)-1-(((S)- tetrahydrofuran-2-yl)methyl)-1H- [1,2,4]triazolo[3,4-b]purine | 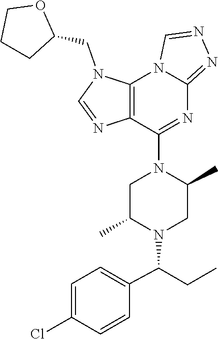 [SMILES: CC[C@H](c1ccc(Cl)cc1)N1C[C@H](C)N(c2nc3nncn3c3c2ncn3C[C@@H]2CCCO2)C[C@H]1C] |
| 4-((2S,5R)-4-((4-chlorophenyl)(3,3- difluorocyclobutyl)methyl)-2,5- dimethylpiperazin-1-yl)-1-(((S)- tetrahydrofuran-2-yl)methyl)-1H- imidazo[4,5-e][1,2,4]triazolo[4,3- a]pyridine | 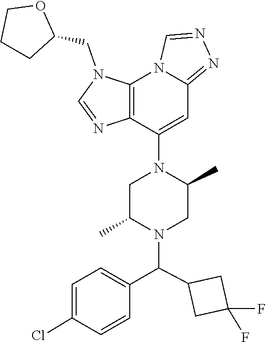 [SMILES: C[C@@H]1CN(c2cc3nncn3c3c2ncn3C[C@@H]2CCCO2)[C@@H](C)CN1C(c1ccc(Cl)cc1)C1CC(F)(F)C1] |
| 4-((2S,5R)-4-((4-chlorophenyl)(3,3- difluorocyclobutyl)methyl)-2,5- dimethylpiperazin-1-yl)-2-methyl-1-(((S)- tetrahydrofuran-2-yl)methyl)-1H- imidazo[4,5-e][1,2,4]triazolo[4,3- a]pyridine | 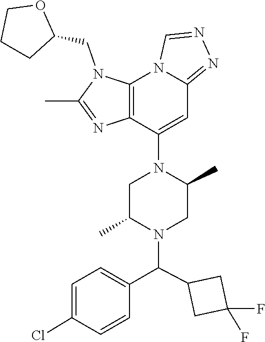 |
| 2-(4-((2S,5R)-4-(Bis(4- fluorophenyl)methyl)-2,5- dimethylpiperazin-1-yl)-1H- [1,2,4]triazolo[3,4-b]purin-1-yl)-N,N- dimethylethan-1-amine | 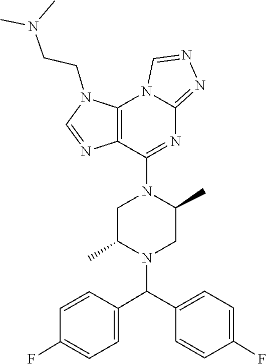 [SMILES: C[C@@H]1CN(c2nc3nncn3c3c2ncn3CCN(C)C)[C@@H](C)CN1C(c1ccc(F)cc1)c1ccc(F)cc1] |

In some embodiments, the DGK inhibitor provided herein is a compound of Formula III:

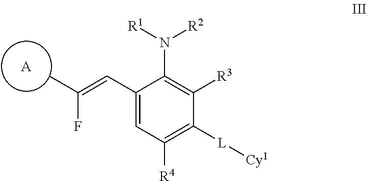

or a pharmaceutically acceptable salt thereof, wherein:
Ring A is:

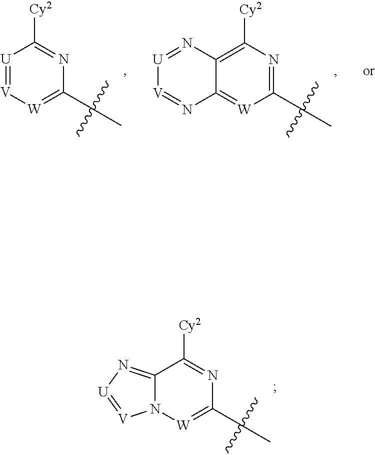

U is CR⁵ or N;
V is CR⁶ or N;
W is CR⁷ or N;
wherein at least one of U, V, or W is not N;
L is a bond, O, or NR¹⁰;
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl are each optionally substituted with 1, 2, 3, or 4 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}NR^{c111}R^{d111}, NR^{c111}C(O)R^{b111}, NR^{e111}C(O)OR^{a111}, NR^{c111}C(O)NR^{c111}R^{d111}, C(═NR^{e111})R^{b111}, C(═NR^{e111})NR^{c111}R^{d111}, NR^{c111}C(═NR^{e111})NR^{c111}R^{d111}, NR^{c111}C(═NR^{e111}) R^{b111}, NR^{c111}S(O)R^{b111}, NR^{e111}S(O)NR^{c111}R^{d111}, NR^{c111}S(O)₂R^{b111}, NR^{c111}S(O)(═NR^{e111}) R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111};
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c111} and R^{d111} attached to the same N atom, together with the N atom to which they are attached, form a 5-10 membered heteroaryl or a 4-10 membered heterocycloalkyl group;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e111} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁-6 alkyl-, (5-10 membered heteroaryl)-C₁₋₆ alkyl-, and (4-10 membered heterocycloalkyl)-C₁₋₆ alkyl-;
Cy² is 4-pyridazinyl, 5 membered heteroaryl, or 4-8 membered heterocycloalkyl, wherein the 4-pyridazinyl, 5 membered heteroaryl, and 4-8 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹ is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{c11})R^{b11}, C(═NR^{c11})NR^{c11}R^{d11}, NR^{c11}C(═NR^{c11})NR^{c11}R^{d11}, NR^{c11}C(═NR^{c11})R^{b11}, NR^{c11}S(O)R^{b11}, NR^{c11}S(O)NR^{c11}R^{d11}, NR^{c11}S(O)₂R^{b11}, NR^{c11}S(O)(═NR^{c11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{c11}, and R^{d11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c11} and R^{d11} attached to the same N atom, together with the N atom to which they are attached, form a 5-10 membered heteroaryl or a 4-10 membered heterocycloalkyl group;
each R^{b11} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
R² is selected from H, C₁₋₆ alkyl, C₁₋₆ haloalkyl, C₂₋₆ alkenyl, C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{c21})R^{b21}, C(═NR^{c21})NR^{c21}R^{d21}, NR^{c21}C(═NR^{c21})NR^{c21}R^{d21}, NR^{c21}C(═NR^{c21})R^{b21}, NR^{c21}S(O)R^{b21}, NR^{c21}S(O)NR^{c21}R^{d21}, NR^{c21}S(O)₂R^{b21}, NR^{c21}S(O)(═NR^{c21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, and OS(O)₂R^{b21};
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or, any R^{c21} and R^{d21} attached to the same N atom, together with the N atom to which they are attached, form a 5-10 membered heteroaryl or a 4-10 membered heterocycloalkyl group;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, or 4 independently selected R^{13A} substituents;
each R^{13A} is independently selected from halo, C₁₋₆ alkyl, C₁₋₆ haloalkyl, C₂₋₆ alkenyl, C₂₋₆ alkynyl, OR^{a132}, and NR^{c132}R^{d132}.
each R^{a132}, R^{c132}, and R^{d132} is independently selected from H and C₁₋₆ alkyl;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, C₃₋₄ cycloalkyl, OH, CN, C(O)OH, C(O)NH₂, and NH₂; wherein the C₁₋₆ alkyl is optionally substituted with OH, CN, and NH₂;
R⁵ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a51}, and NHR^{a52} wherein the C₁₋₆ alkyl is optionally substituted with OH, CN, and NH₂;
each R^{a51} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
each R^{a52} is independently selected from C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R⁶ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, and C(O)NH₂; wherein the C₁₋₆ alkyl is optionally substituted with OH, CN, and NH₂; and
R⁷ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NHR^{a71}, and NHR^{a72}, wherein the C₁₋₆ alkyl is optionally substituted with OH, CN, and NH₂;
R¹⁰ is selected from H, halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OH, CN, C(O)OH, C(O)NH₂, and NH₂; wherein the C₁₋₆ alkyl, C₂₋₆ alkenyl, and C₂₋₆ alkynyl of R¹⁰ are each optionally substituted with 1, 2, 3, or 4 independently selected R^{M} substituents; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl.

In some embodiments, the DGK inhibitor provided herein is a compound of Formula IV:

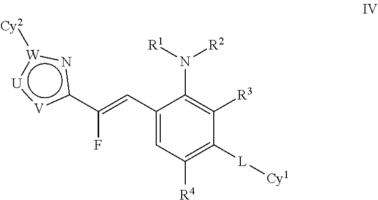

or a pharmaceutically acceptable salt thereof, wherein:
U is CR⁵ or S;
V is CR⁶;
W is C or N;
L is a bond, O, or NR¹⁰;
Cy¹ is selected from phenyl, 5-6 membered heteroaryl, C₃₋₇ cycloalkyl, and 4-7 membered heterocycloalkyl, wherein the phenyl, 5-6 membered heteroaryl, C₃₋₇ cycloalkyl, and 4-7 membered heterocycloalkyl of Cy¹ are each optionally substituted with 1, 2, 3, or 4 independently selected R¹¹ substituents;
each R¹¹ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a111}, SR^{a111}, NHOR^{a111}, C(O)R^{b111}, C(O)NR^{c111}R^{d111}, C(O)NR^{c111}(OR^{a111}), C(O)OR^{a111}, OC(O)R^{b111}, OC(O)NR^{c111}R^{d111}, NR^{c111}R^{d111}, NR^{c111}NR^{c111}R^{d111}NR^{c111}C(O)R^{b111}, NR^{c111}C(O)OR^{a111}, NR^{c111}C(O)NR^{c111}R^{d111}, C(═NR^{c111})R^{b111}, C(═NR^{e111})NR^{c111}R^{d111}, NR^{c111}C(═NR^{c111})NR^{c111}R^{d111}, NR^{c111}C(═NR^{c111})R^{b111}, NR^{c111}S(O)R^{b111}, NR^{c111}S(O)NR^{c111}R^{d111}, NR^{c111}S(O)₂R^{b111}, NR^{c111}S(O)(═NR^{c111})R^{b111}, NR^{c111}S(O)₂NR^{c111}R^{d111}, S(O)R^{b111}, S(O)NR^{c111}R^{d111}, S(O)₂R^{b111}, S(O)₂NR^{c111}R^{d111}, OS(O)(═NR^{e111})R^{b111}, and OS(O)₂R^{b111}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹¹ are each optionally substituted with 1, 2, 3, 4, 5, 6, 7, or 8 independently selected R^{11A} substituents;
each R^{a111}, R^{c111}, and R^{d111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a111}, R^{c111} and R^{d111} are each optionally substituted with 1, 2, 3, 4, 5, 6, 7, or 8 independently selected R^{11A} substituents;
or, any R^{c111} and R^{d111} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{11A} substituents;
each R^{b111} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b111} are each optionally substituted with 1, 2, 3, 4, 5, 6, 7, or 8 independently selected R^{11A} substituents;
each R^{e111} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{11A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a112}, C(O)NR^{c112}R^{d112}, C(O)OR^{a112}, NR^{c112}R^{d112}, S(O)NR^{c112}R^{d112}, S(O)₂R^{b112} S(O)₂NR^{c112}R^{d112}, and OS(O)₂R^{b112}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{11A} are each optionally substituted with 1, 2, 3, or 4 independently selected R^{M} substituents;
each R^{a112}, R^{c112} and R^{d112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a112}, R^{c112} and R^{d112} are each optionally substituted with 1, 2, 3, or 4 independently selected R^{M} substituents;
or, any R^{c112} and R^{d112} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b112} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b112} are each optionally substituted with 1, 2, 3, or 4 independently selected R^{M} substituents;
Cy² is 4-pyridazinyl, 5 membered heteroaryl, or 4-6 membered heterocycloalkyl, wherein the 4-pyridazinyl, 5 membered heteroaryl, and 4-6 membered heterocycloalkyl are each optionally substituted with 1, 2, 3, or 4 independently selected R¹² substituents;
R¹² is selected from halo, C₁₋₆ alkyl, C₁₋₆ alkoxy, C₁₋₆ haloalkyl, C₁₋₆ haloalkoxy, C₂₋₆ alkenyl, C₂₋₆ alkynyl, OR^{a121}, CN, C(O)OH, C(O)NHR^{a121}, and NR^{a121}R^{a121}, wherein the C₁₋₆ alkyl is optionally substituted with OH, CN, and NH₂;
each R^{a121} is independently selected from H, C₁₋₆ alkyl, C₁₋₆ alkoxy, C₁₋₆ haloalkyl, C₁₋₆ haloalkoxy, OH, NH₂, and NHC₁₋₆ alkyl, wherein the C₁₋₆ alkyl is optionally substituted with OH, CN, and NH₂;
R¹ is selected from C₁₋₆ alkyl, C₁₋₆ haloalkyl, C₆₋₁₀ aryl, C₃₋₁₀ cycloalkyl, 5-10 membered heteroaryl, and 4-10 membered heterocycloalkyl, wherein the C₁₋₆ alkyl, C₆₋₁₀ aryl, C₃₋₁₀ cycloalkyl, 5-10 membered heteroaryl, and 4-10 membered heterocycloalkyl of R¹ are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, and C₂₋₆ alkynyl;
R² is selected from C₁₋₆ alkyl, C₁₋₆ haloalkyl, C₆₋₁₀ aryl, C₃₋₁₀ cycloalkyl, 5-10 membered heteroaryl, and 4-10 membered heterocycloalkyl, wherein the C₁₋₆ alkyl, C₆₋₁₀ aryl, C₃₋₁₀ cycloalkyl, 5-10 membered heteroaryl, and 4-10 membered heterocycloalkyl of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, and C₂₋₆ alkynyl;
or R¹ and R², together with the N atom to which they are attached, form a 4-12 membered heterocycloalkyl, wherein the 4-12 membered heterocycloalkyl is optionally substituted with 1, 2, 3, or 4 independently selected R¹³ substituents;
each R¹³ is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a131}, SR^{a131}, NHOR^{a131}, C(O)R^{b131}, C(O)NR^{c131}R^{d131}, C(O)NR^{c131} (OR^{a131}), C(O)OR^{a131}, OC(O)R^{b131}, OC(O)NR^{c131}R^{d131}, NR^{c131}R^{d131}, NR^{c131}NR^{c131}R^{d131}, NR^{c131}C(O)R^{b131}, NR^{c131}C(O)OR^{a131}, NR^{e131}C(O)NR^{c131}R^{d131}, C(═NR^{e131}) R^{b131}, C(═NR^{e131}) NR^{c131}R^{d131}, C(═NOR^{a131}) R^{b131}, C(═NOR^{a131}) OR^{a131}, NR^{c131}C(═NR^{e131}) NR^{c131}R^{d131}, NR^{c131}C(—NR^{e131}) R^{b131}, NR^{c131}S(O)R^{b131}, NR^{c131}S(O)NR^{c131}R^{d131}, NR^{c131}S(O)₂R^{b131}, NR^{c131}S(O)(═NR^{e131}) R^{b131}, NR^{c131}S(O)₂NR^{c131}R^{d131}, S(O)R^{b131}, S(O)NR^{c131}R^{d131}, S(O)₂R^{b131}, S(O)₂NR^{c131}R^{d131}, OS(O)(═NR^{e131})R^{b131}, and OS(O)₂R^{b13}1, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R¹³ are each optionally substituted with 1, 2, 3, 4, 5, 6, 7, or 8 independently selected R^{13A} substituents;
each R^{a131}, R^{c131}, and R^{d131} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a131}, R^{c131} and R^{d131} are each optionally substituted with 1, 2, 3, 4, 5, 6, 7, or 8 independently selected R^{13A} substituents;
or, any R^{c131} and R^{d131} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13A} substituents;
each R^{b131} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b131} are each optionally substituted with 1, 2, 3, 4, 5, 6, 7, or 8 independently selected R^{13A} substituents;
each R^{e131} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13A} is independently selected from halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a132}, SR^{a132}, NHOR^{a132}, C(O)R^{b132}, C(O)NR^{c132}R^{d132}, C(O)NR^{c132}(OR^{a132}), C(O)OR^{a132}, OC(O)R^{b132}, OC(O)NR^{c132}R^{d132} NR^{e132}R^{d132} NR^{c132}NR^{e132}R^{d132}, NR^{c132}C(O)R^{b132} NR^{c132}C(O)OR^{a132}, NR^{c132}C(O)NR^{c132}R^{d132}, C(═NR^{e132})R^{b132}, C(═NR^{e132})NR^{c132}R^{d132} NR^{c132}C(═NR^{e132})NR^{c132}R^{d132} NR^{c132}C(═NR^{e132})R^{b132}, NR^{c132}S(O)R^{b132} NR^{c132}S(O)NR^{c132}R^{d132} NR^{e132}S(O)₂R^{b132} NR^{e132}S(O)(═NR^{e132})R^{b132}, NR^{c132}S(O)₂NR^{e132}R^{d132} S(O)R^{b132}, S(O)NR^{c132}R^{d132}, S(O)₂R^{b132}, S(O)₂NR^{c132}R^{d132} OS(O)(═NR^{e132})R^{b132}, and OS(O)₂R^{b132}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{13A} are each optionally substituted with 1, 2, 3, 4, 5, 6, 7, or 8 independently selected R^{13B} substituents;
each R^{a132}, R^{c132}, and R^{d132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a132}, R^{c132} and R^{d132} are each optionally substituted with 1, 2, 3, 4, 5, 6, 7, or 8 independently selected R^{13B} substituents;
or, any R^{c132} and R^{d132} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{13B} substituents;
each R^{b132} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b132} are each optionally substituted with 1, 2, 3, 4, 5, 6, 7, or 8 independently selected R^{13B} substituents;
each R^{e132} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{13B} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a133}, C(O)NR^{c133}R^{d133}, C(O)OR^{a113}, NR^{c113}R^{d133}, S(O)NR^{c133}R^{d133}, S(O)₂R^{b133}, S(O)₂NR^{c133}R^{d133}, and OS(O)₂R^{b133}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, of R^{13B} are each optionally substituted with 1, 2, 3, or 4 independently selected R^{M} substituents;
each R^{a133}, R^{c133}, and R^{d133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a133}, R^{c133} and R^{d133} are each optionally substituted with 1, 2, 3, or 4 independently selected R^{M} substituents;
or, any R^{c33} and R^{d133} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b133} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b133} are each optionally substituted with 1, 2, 3, or 4 independently selected R^{M} substituents;
R³ is selected from H, halo, C₁₋₆ alkyl and C₁₋₆ haloalkyl;
R⁴ is selected from H, halo, C₁₋₆ alkyl, C₁₋₆ alkoxy, C₁₋₆ haloalkyl, and C₁₋₆ haloalkoxy;
R⁵ is selected from H, C₁₋₆ alkyl, and C₁₋₆ haloalkyl;
R⁶ is selected from H, C₁₋₆ alkyl, and C₁₋₆ haloalkyl;
R¹⁰ is selected from H, C₁₋₆ alkyl, and C₁₋₆ haloalkyl; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-.

In some embodiments, the DGK inhibitor provided herein is a compound of Formula V:

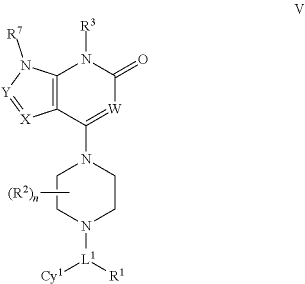

or a pharmaceutically acceptable salt thereof, wherein:
W is CR⁴ or N;
X is CR⁵ or N;
Y is CR⁶ or N;
n is 1, 2, or 3;
L¹ is C₁₋₃ alkyl;
R¹ is C₆₋₁₀ aryl or C₃₋₁₀ cycloalkyl, wherein the C₆₋₁₀ aryl and C₃₋₁₀ cycloalkyl are each optionally substituted with 1, 2, 3, or 4 independently selected R^{1A} substituents;
each R^{1A} is independently selected from halo, C₁₋₆ alkyl, C₁₋₆ haloalkyl, C₂₋₆ alkenyl, and C₂₋₆ alkynyl;
each R² is independently selected from C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R² are each optionally substituted with 1, 2, 3, or 4 independently selected R^{M} substituents;
R³ is selected from H, halo, C₁₋₆ alkyl, C₁₋₆ haloalkyl, C₂₋₆ alkenyl, and C₂₋₆ alkynyl;
R⁴ is selected from H, halo, C₁₋₆ alkyl, C₁₋₆ haloalkyl, C₂₋₆ alkenyl, and C₂₋₆ alkynyl;
R⁵ is selected from H, halo, C₁₋₆ alkyl, C₁₋₆ haloalkyl, C₂₋₆ alkenyl, and C₂₋₆ alkynyl;
R⁶ is selected from H, halo, C₁₋₆ alkyl, C₁₋₆ haloalkyl, C₂₋₆ alkenyl, and C₂₋₆ alkynyl;
R⁷ is selected from C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
Cy¹ is C₆₋₁₀ aryl or C₃₋₁₀ cycloalkyl, wherein the C₆₋₁₀ aryl and C₃₋₁₀ cycloalkyl are each optionally substituted with 1, 2, 3, or 4 independently selected R⁸ substituents;
each R⁸ is independently selected from halo, C₁₋₆ alkyl, C₁₋₆ haloalkyl, C₂₋₆ alkenyl, and C₂₋₆ alkynyl; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, and C₂₋₆ alkynyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the terms “Cₙ₋ₘ” and “Cₘ₋ₙ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, from 2 to 6 carbon atoms, from 2 to 4 carbon atoms, from 2 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, and the like.

In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-, 7-, 8-, 9-, or, 10-membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-, 7-, 8-, 9-, or 10-membered monocyclic or bicyclic heteroaryl having 1, 2, 3, or 4 heteroatom ring members independently selected from N, O, and S. In some embodiments, the heteroaryl is a 5-6 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, S, and B. In some embodiments, the heteroaryl is a 5 membered monocyclic heteroaryl having 1 or 2 heteroatom ring members independently selected from N, O, and S. In some embodiments, the heteroaryl group contains 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl, 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring.

In some embodiments, the heterocycloalkyl group contains 3 to 10 ring-forming atoms, 4 to 10 ring-forming atoms, 4 to 8 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10, membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, tetrahydrothiopheneyl, tetrahydrothiopheneyl 1,1-dioxide, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, 2-azaspiro[3.3]heptanyl, diazaspiro[3.3]heptanyl, azaspiro[3.5]nonanyl, 7-azaspiro[3.5]nonanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxo-pyrrolidinyl, oxo-pyridinyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkyl linking group” or “alkylene linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-diyl, propan-1,2-diyl, propan-1,1-diyl and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula II, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The schemes below provide general guidance in connection with preparing the DGK inhibitors of the invention. One skilled in the art would understand that the preparations shown in the schemes can be modified or optimized using general knowledge of organic chemistry to prepare various compounds of the invention.

Compounds of Formula I and II can be synthesized, for example, using the process shown in Scheme 1. As depicted in Scheme 1, a number of methods (e.g., nucleophilic aromatic substitution or a suitable cross-coupling reaction) can be used to access compounds of the general Formula 1-2. For example, compounds of Formula 1-1 (i.e., each Hal can independently be F, Cl, Br, or I) can be reacted with an appropriate amine nucleophile in an appropriate solvent (e.g., 1-butanol) at an appropriate temperature (e.g., ranging from room temperature to 200° C.) for a suitable time (e.g., ranging from several minutes to several days) to generate compounds of Formula 1-2. Alternatively, transition metal (e.g., Pd, Cu, Ni) catalyzed reactions (including, but not limited to, Buchwald, Ullman, Suzuki, Stille, Negishi couplings) of compounds 1-1 and appropriate coupling partners (e.g., primary or secondary amines, nitrogen heterocycles, or heteroaryl boronic acids/esters, trialkyl tin, or zinc reagents) affords compounds of Formula 1-2. Compounds of Formula 1-1 are commercially available, or can be readily synthesized according to methods known by persons skilled in the art. C—N bond forming reactions (e.g., transition metal catalyzed or nucleophilic aromatic substitution) between compounds of Formula 1-2 and hydrazine under appropriate conditions (e.g., in the presence of a palladium catalyst, such as methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (“tBuBrettPhos Pd G3”), and a base, such as Cs₂CO₃ or NaOt-Bu, in an appropriate solvent, such as THE or 1,4-dioxane) generates compounds of Formula 1-3. Reaction of compounds of Formula 1-3 with compounds of Formula 1-4 (e.g., trimethyl orthoformate or triethyl orthoacetate) under appropriate conditions (e.g., in the presence of AcOH) provides compounds of Formula I or II.

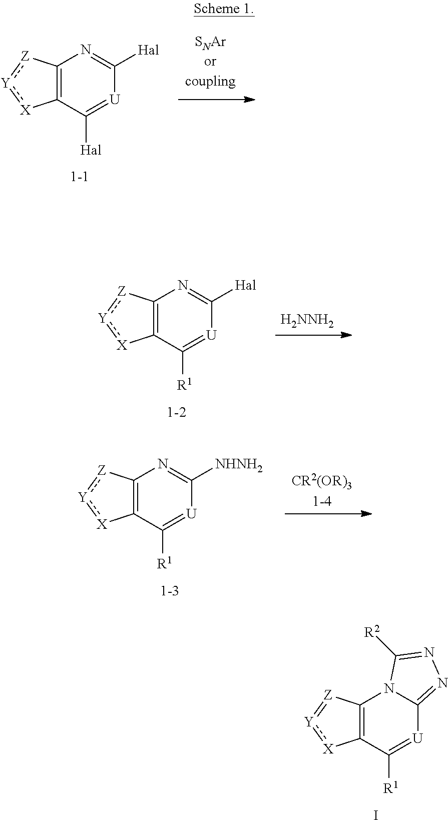

Compounds of Formula I or II can also be prepared, for example, using the process illustrated in Scheme 2. As depicted in Scheme 2, compounds of Formula 2-1 can be converted into compounds of Formula 2-2 by a number of methods. For example, halogenation of compounds of Formula 2-1 (e.g., via deprotonation with an appropriate base, such as 2,2,6,6-tetramethylpiperidinylmagnesium chloride lithium chloride (“TMPMgCl·LiCl”), followed by addition of an appropriate electrophile, such as 1-chloro-2-iodoethane) followed by a suitable cross-coupling affords compounds of Formula 2-2. Examples of suitable cross-coupling reactions include, but are not limited to, Suzuki (see e.g., Tetrahedron 2002, 58, 9633-9695), Negishi (see e.g., ACS Catalysis 2016, 6, 1540-1552), Stille (see e.g., ACS Catalysis 2015, 5, 3040-3053), Sonogashira (see e.g., Chem. Soc. Rev. 2011, 40, 5084-5121), Buchwald-Hartwig amination (see e.g., Chem. Sci. 2011, 2, 27-50), Cu-catalyzed amination (see e.g., Org. React. 2014, 85, 1-688), among others. Alternatively, compounds of Formula 2-2 can be accessed by conversion of compounds of Formula 2-1 to a carbonyl intermediate (e.g., by deprotonation with an appropriate base, such as TMPMgCl·LiCl, followed by addition of an appropriate electrophile, such as DMF) followed by reaction with a suitable fluorinating reagent (e.g., diethylaminosulfur trifluoride). C—N bond forming reactions (e.g., transition metal catalyzed or nucleophilic aromatic substitution) between compounds of Formula 2-2 and hydrazine under appropriate conditions (e.g., in the presence of a palladacycle precatalyst, such as tBuBrettPhos Pd G3, and a base, such as Cs₂CO₃) generates compounds of Formula 2-2. Reaction of compounds of Formula 2-2 with compounds of Formula 2-3 (e.g., triethyl orthoformate) under appropriate conditions (e.g., in the presence of AcOH) provides compounds of Formula I or II.

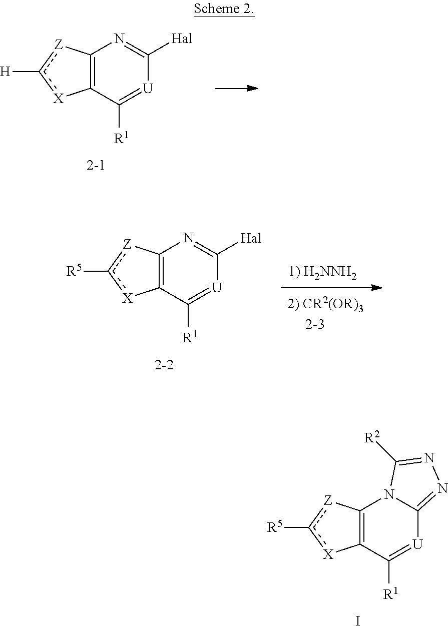

Compounds of Formula 3-8 can be synthesized, for example, according to the process shown in Scheme 3. As depicted in Scheme 3, protection of amino compounds of Formula 3-1 under appropriate conditions (e.g., including, but not limited to, reductive amination reactions with an appropriate aldehyde, such as benzaldehyde, in the presence of a reducing agent, such as sodium triacetoxyborohydride) generates compounds of Formula 3-2. Compounds of Formula 3-1 are commercially available, or can be readily synthesized according to methods known by persons skilled in the art. Amide coupling reactions of compounds of Formula 3-2 with compounds of Formula 3-3 under suitable conditions (e.g., in the presence of a coupling reagent, such as HATU, and a base, such as N-ethyl-N-isopropylpropan-2-amine, in an appropriate solvent, such as N,N-dimethylformamide) affords compounds of Formula 3-4. Deprotection of the tert-butyloxycarbonyl group in compounds of Formula 3-4 under appropriate conditions (e.g., using an acid, such as trifluoroacetic acid), followed by intramolecular cyclization under appropriate conditions (e.g., using a suitable solvent, such as MeOH) provides compounds of Formula 3-5. Reduction of compounds of Formula 3-5 under suitable conditions (e.g., using a reducing agent, such as borane, in a suitable solvent, such as THF) generates compounds of Formula 3-6. Protection of compounds of Formula 3-6 under appropriate conditions (e.g., via reaction with di-tert-butyl dicarbonate in the presence of a base, such as N-ethyl-N-isopropylpropan-2-amine) provides compounds of Formula 3-7. Selective deprotection of PG in compounds of Formula 3-7 (e.g., where PG is a protecting group such as benzyl) under appropriate conditions (e.g., using an appropriate catalyst, such as palladium on carbon, in the presence of hydrogen gas), affords compounds of Formula 3-8.

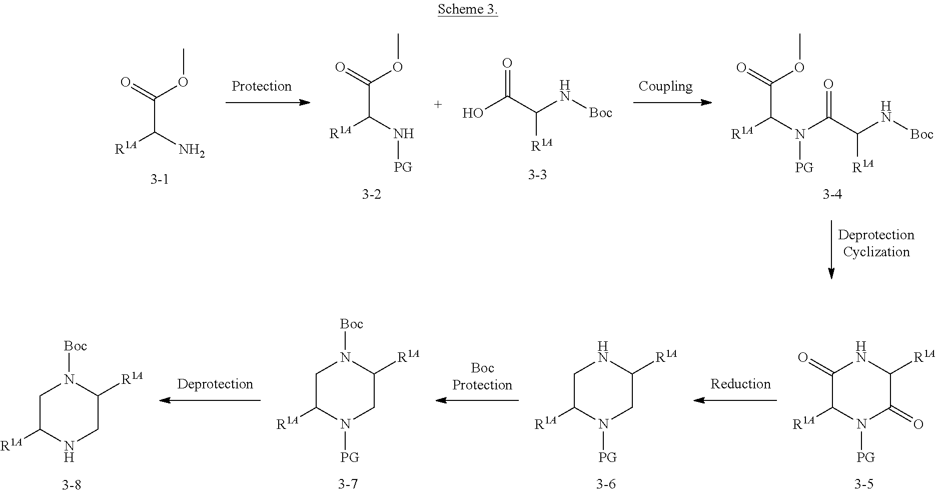

Compounds of Formula 4-4 can be prepared, for example, using the process illustrated in Scheme 4. In the process depicted in Scheme 4, nucleophilic substitution reactions between compounds of Formula 4-1 and compounds of Formula 4-2 under appropriate conditions (e.g., in the presence of a base, such as N-ethyl-N-isopropylpropan-2-amine, in an appropriate solvent, such as CH₃CN) generates compounds of Formula 4-3. Removal of an appropriate protecting group (e.g., wherein PG is a group such as tert-butoxycarbonyl) from compounds of Formula 4-3 under appropriate conditions (e.g., in the presence of an acid, such as HCl or trifluoroacetic acid, in a suitable solvent, such as tetrahydrofuran, 1-4-dioxane, or CH₂Cl₂) affords compounds of Formula 4-4.

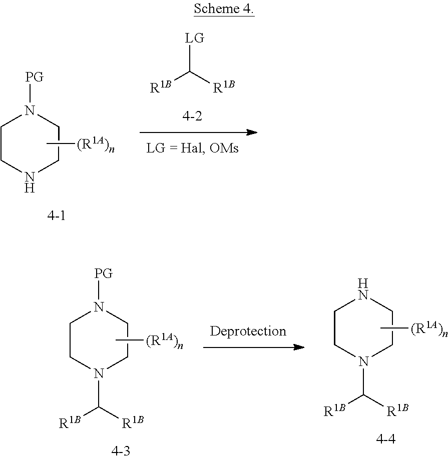

Alternatively, compounds of Formula 4-4 can be prepared, for example, using the process illustrated in Scheme 5. In the process depicted in Scheme 5, amide coupling reactions of compounds of Formula 5-1 with compounds of Formula 5-2 affords compounds of Formula 5-3. Subjection of compounds of Formula 5-3 to reductive alkylation conditions (e.g., through the use of an appropriate transition metal catalyst, such as IrCl(CO)(PPh₃)₂, in the presence of a silane, such as 1,1,3,3-tetramethyldisiloxane, followed by addition of a suitable organometallic reagent, such as a Grignard reagent) affords compounds of Formula 5-4. Removal of an appropriate protecting group (e.g., wherein PG is a group such as tert-butoxycarbonyl) from compounds of Formula 5-4 under appropriate conditions (e.g., in the presence of an acid, such as HCl or trifluoroacetic acid, in a suitable solvent, such as tetrahydrofuran, 1-4-dioxane, or CH₂Cl₂) affords compounds of Formula 4-4.

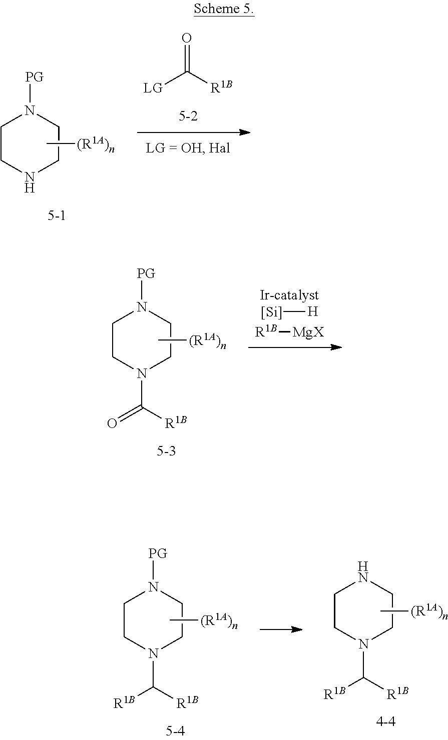

Compounds of Formula III can be synthesized, for example, using the process shown in Scheme 6. As depicted in Scheme 6, nucleophilic aromatic substitution reactions of compounds of Formula 6-1 (e.g., wherein the Halo² group is a fluorine and the Halo¹ group is a chloro, bromo, or iodo) under appropriate conditions (e.g., in the presence of base, such as Cs₂CO₃) generates compounds of Formula 6-2. Alternatively, transition-metal (e.g., Pd, Cu, Ni) catalyzed coupling reactions (including, but not limited to, Buchwald-Hartwig, Ullman, Suzuki, Stille, and Negishi couplings) between compounds of Formula 6-1 (e.g., wherein the Halo² group is a chloro, bromo, or iodo) and appropriate coupling partners generates compounds of Formula 6-2. Compounds of Formula 6-1 are commercially available, or can be readily synthesized according to methods known by persons skilled in the art. Nucleophilic aromatic substitution reactions of compounds of Formula 6-2 with appropriate amine nucleophiles under appropriate conditions (e.g., in the presence of a base, such as N-ethyl-N-isopropylpropan-2-amine, in an appropriate solvent, such as CH₃CN) affords compounds of Formula 6-3. Alternatively, transition metal catalyzed couplings (including, but not limited to, Buchwald-Hartwig couplings) between compounds of Formula 6-2 and appropriately substituted amines affords compounds of Formula 6-3. Reduction of the nitro group to compounds of Formula 6-3 under suitable conditions (e.g., using iron as reductant in the presence of an additive, such as NH₄Cl, in an appropriate solvent mixture, such as THF/MeOH/H₂O), followed by conversion to an iodide (e.g., via Sandmeyer reaction) provides compounds of Formula 6-4. Suzuki coupling of the iodide group in compounds of Formula 6-4 with an appropriate 2,2-difluorovinyl boronic ester (i.e., 2-(2,2-difluorovinyl)-4,4,5,5-tetramethyl-1,3,2-dioxaborolane) under appropriate conditions (e.g., in the presence of a palladium catalyst, such as [1,1′-bis(diphenylphosphino)ferrocene]dichloropalladium (II), and a base, such as Cs₂CO₃) provides compounds of Formula 6-5. Copper-catalyzed regioselective monodefluoroborylation of compounds of Formula 6-5 under appropriate conditions (e.g., such as described in J. Am. Chem. Soc. 2017, 139, 12855-12862) provides compounds of Formula 6-6. Finally, compounds of Formula III can be synthesized by transition metal catalyzed coupling reactions (including, but not limited to, Suzuki, Chan-Lam couplings) of compounds of Formula 6-6 with an appropriately substituted heteroaromatic coupling partner.

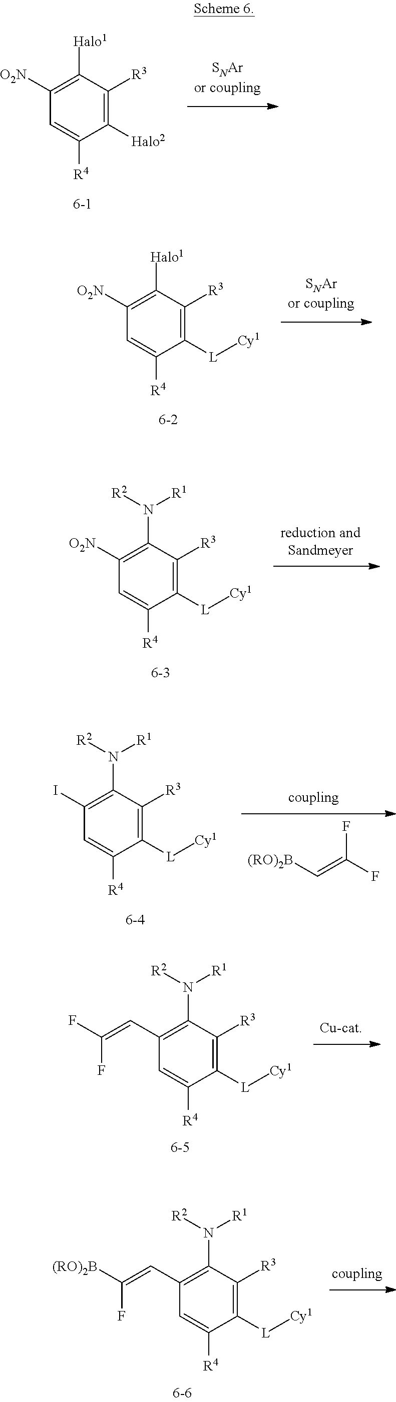

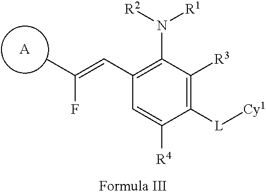

Alternatively, compounds of Formula 6-3 can be synthesized, for example, using the process shown in Scheme 7. As depicted in Scheme 7, nucleophilic aromatic substitution reactions of compounds of Formula 7-1 (e.g., wherein the Halo¹ group is a fluorine and the Halo² group is a chloro, bromo, or iodo) under appropriate conditions (e.g., in the presence of a base, such as N-ethyl-N-isopropylpropan-2-amine) generates compounds of Formula 7-2. Alternatively, transition metal catalyzed couplings (including, but not limited to, Buchwald-Hartwig couplings) between compounds of Formula 7-1 (e.g., wherein the Halo¹ group is a chloro, bromo, or iodo) and appropriately substituted amines affords compounds of Formula 7-2. Compounds of Formula 7-1 are commercially available, or can be readily synthesized according to methods known by persons skilled in the art. Nucleophilic aromatic substitution reactions of compounds of Formula 7-2 with appropriate amine nucleophiles under appropriate conditions (e.g., in the presence of base, such as Cs₂CO₃, in an appropriate solvent, such as CH₃CN) affords compounds of Formula 6-3. Alternatively, transition-metal (e.g., Pd, Cu, Ni) catalyzed coupling reactions (including, but not limited to, Buchwald-Hartwig, Ullman, Suzuki, Stille, and Negishi couplings) between compounds of Formula 7-2 and appropriate coupling partners under suitable conditions generates compounds of Formula 6-3. Compounds of Formula III can be synthesized from compounds of Formula 6-3 via the process shown in Scheme 6.

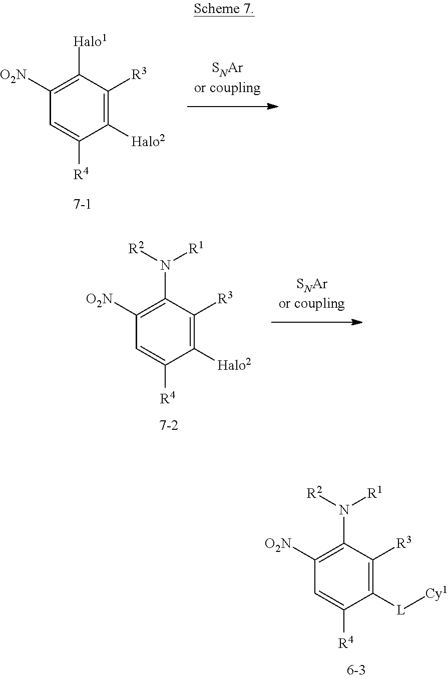

Compounds of Formula IV can be synthesized, for example, using the process shown in Scheme 8. As depicted in Scheme 8, nucleophilic aromatic substitution reactions of compounds of Formula 8-1 (e.g., wherein the Halo² group is a fluorine and the Halo¹ group is a chloro, bromo, or iodo) under appropriate conditions (e.g., in the presence of base, such as Cs₂CO₃) generates compounds of Formula 8-2. Alternatively, transition-metal (e.g., Pd, Cu, Ni) catalyzed coupling reactions (including, but not limited to, Buchwald-Hartwig, Ullman, Suzuki, Stille, and Negishi couplings) between compounds of Formula 8-1 (e.g., wherein the Halo² group is a chloro, bromo, or iodo) and appropriate coupling partners generates compounds of Formula 8-2. Compounds of Formula 8-1 are commercially available, or can be readily synthesized according to methods known by persons skilled in the art. Nucleophilic aromatic substitution reactions of compounds of Formula 8-2 with appropriate amine nucleophiles under appropriate conditions (e.g., in the presence of a base, such as N-ethyl-N-isopropylpropan-2-amine, in an appropriate solvent, such as CH₃CN) affords compounds of Formula 6-3. Alternatively, transition metal catalyzed couplings (including, but not limited to, Buchwald-Hartwig couplings) between compounds of Formula 8-2 and appropriately substituted amines affords compounds of Formula 6-3. Alternatively, compounds of Formula 6-3 in Scheme 8 can be prepared according to the synthesis of Scheme 7. Reduction of the nitro group to compounds of Formula 6-3 under suitable conditions (e.g., using iron as reductant in the presence of an additive, such as NH₄Cl, in an appropriate solvent mixture, such as THF/MeOH/H₂O), followed by conversion to an iodide (e.g., via Sandmeyer reaction) provides compounds of Formula 8-4. Suzuki coupling of the iodide group in compounds of Formula 8-4 with an appropriate 2,2-difluorovinyl boronic ester (i.e., 2-(2,2-difluorovinyl)-4,4,5,5-tetramethyl-1,3,2-dioxaborolane) under appropriate conditions (e.g., in the presence of a palladium catalyst, such as [1,1′-bis(diphenylphosphino)ferrocene]dichloropalladium (II), and a base, such as Cs₂CO₃) provides compounds of Formula 8-5. Copper-catalyzed regioselective monodefluoroborylation of compounds of Formula 8-5 under appropriate conditions (e.g., such as described in J. Am. Chem. Soc. 2017, 139, 12855-12862) provides compounds of Formula 8-6. Finally, compounds of Formula IV can be synthesized by transition metal catalyzed coupling reactions (including, but not limited to, Suzuki, Chan-Lam couplings) of compounds of Formula 8-6 with an appropriately substituted heteroaromatic coupling partner.

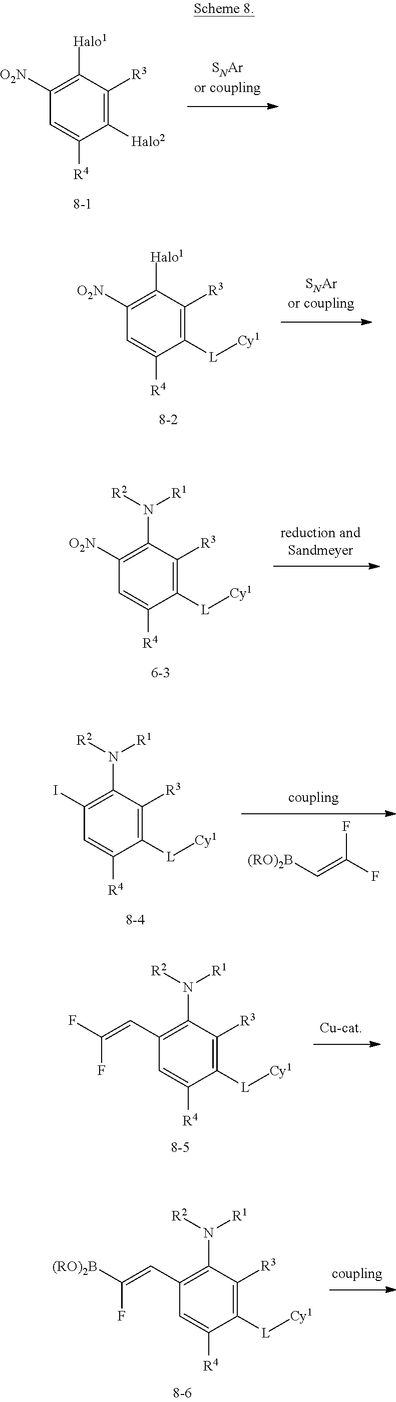

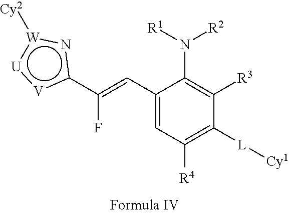

Compounds of Formula 9-4 can be prepared, for example, using the process illustrated in Scheme 9. In the process depicted in Scheme 9, nucleophilic substitution reactions between compounds of Formula 9-1 and compounds of Formula 9-2 under appropriate conditions (e.g., in the presence of a base, such as N-ethyl-N-isopropylpropan-2-amine, in an appropriate solvent, such as CH₃CN) generates compounds of Formula 9-3. Removal of an appropriate protecting group (e.g., wherein PG is a group such as tert-butoxycarbonyl) from compounds of Formula 9-3 under appropriate conditions (e.g., in the presence of an acid, such as HCl or trifluoroacetic acid, in a suitable solvent, such as tetrahydrofuran, 1,4-dioxane, or CH₂Cl₂) affords compounds of Formula 9-4.

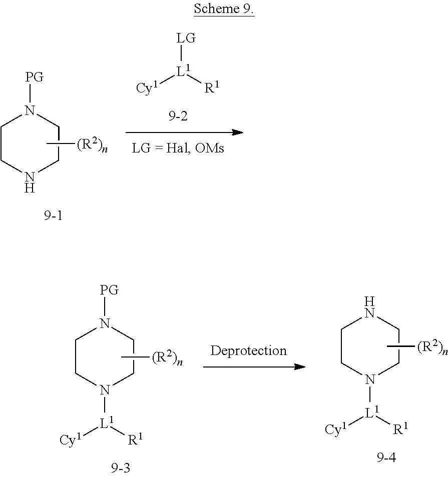

Alternatively, compounds of Formula 9-4 can be prepared, for example, using the process illustrated in Scheme 10. In the process depicted in Scheme 10, amide coupling reactions of compounds of Formula 10-1 with compounds of Formula 10-2 affords compounds of Formula 10-3. Subjection of compounds of Formula 10-3 to reductive alkylation conditions (e.g., through the use of an appropriate transition metal catalyst, such as IrCl(CO)(PPh₃)₂, in the presence of a silane, such as 1,1,3,3-tetramethyldisiloxane, followed by addition of a suitable organometallic reagent, such as a Grignard reagent) affords compounds of Formula 9-3. Removal of an appropriate protecting group (e.g., wherein PG is a group such as tert-butoxycarbonyl) from compounds of Formula 9-3 under appropriate conditions (e.g., in the presence of an acid, such as HCl or trifluoroacetic acid, in a suitable solvent, such as tetrahydrofuran, 1,4-dioxane, or CH₂Cl₂) affords compounds of Formula 9-4. For earlier introduction of the Cy¹ substituent, amide coupling of compounds of Formula 10-1 with compounds of Formula 10-4 affords compounds of Formula 10-5. Subsequent installation of the R¹ substituent can be achieved by subjection of compounds of Formula 10-5 to reductive alkylation conditions to furnish intermediate compounds of Formula 9-3.

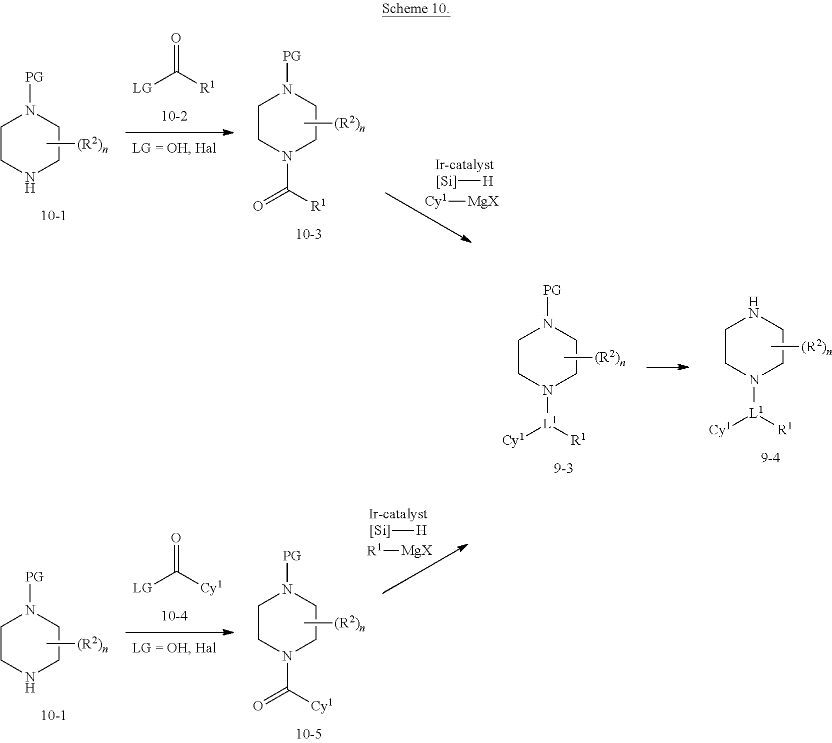

Compounds of Formula 11-4 can be synthesized using the process shown in Scheme 11. As depicted in Scheme 11, a number of methods (e.g., nucleophilic aromatic substitution or a suitable cross-coupling reaction) can be used to access compounds of the general Formula 11-2. For example, compounds of Formula 11-1 (i.e., each Hal can independently be F, Cl, Br, or I) can be reacted with an appropriate amine nucleophile 9-4 in an appropriate solvent (e.g., 1-butanol) at an appropriate temperature (e.g., ranging from room temperature to 200° C.) for a suitable time (e.g., ranging from several minutes to several days) to generate compounds of Formula 11-2. Alternatively, transition metal (e.g., Pd, Cu, Ni) catalyzed reactions (including, but not limited to, Buchwald, Ullman, Suzuki, Stille, Negishi couplings) of compounds 11-1 and appropriate coupling partners (e.g., primary or secondary amines, nitrogen heterocycles, or heteroaryl boronic acids/esters, trialkyl tin, or zinc reagents) affords compounds of Formula 11-2. Compounds of Formula 11-1 are commercially available, or can be readily synthesized according to methods known by persons skilled in the art. Nitrogen functionalization of compounds of Formula 11-2 using a number of methods (e.g., including, but not limited to, nucleophilic substitution or Mitsunobu reactions) provides access into compounds of Formula 11-4. For example, compounds of Formula 11-2 can be reacted with an appropriate electrophile (e.g., (S)-(tetrahydrofuran-2-yl)methyl methanesulfonate) in the presence of a suitable base (e.g., potassium carbonate) to afford compounds of Formula 11-4. Alternatively, direct functionalization of compounds of Formula 11-1 using a number of methods (e.g., including, but not limited to, nucleophilic substitution or Mitsunobu reactions) provides access into compounds of Formula 11-3. For example, compounds of Formula 11-1 can be reacted with a suitable alcohol (e.g., (S)-(tetrahydrofuran-2-yl)methanol) in the presence of appropriate reagents (e.g., including a phosphine, such as triphenylphosphine, and an azodicarboxylate, such as diisopropyl azodicarboxylate) to furnish compounds of Formula 11-3. Reaction of compounds of Formula 11-3 with amine nucleophiles of Formula 9-4 using a number of methods (e.g., nucleophilic aromatic substitution or a suitable cross-coupling reaction) can be used to access compounds of Formula 11-4.

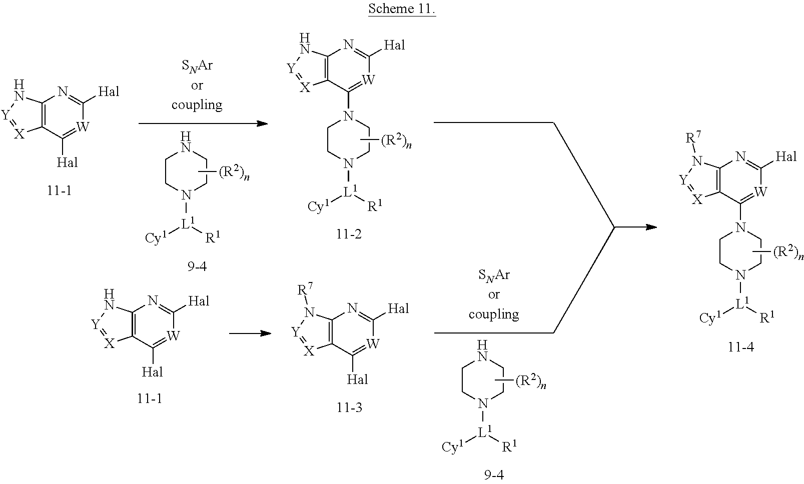

Compounds of Formula V can be prepared using the process illustrated in Scheme 12. As depicted in Scheme 12, C—O bond forming reactions (e.g., transition metal catalyzed or nucleophilic aromatic substitution) between compounds of Formula 11-4 and an appropriate nucleophile (e.g., potassium hydroxide) under appropriate conditions (e.g., in the presence of a palladium catalyst, such as methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II)) in an appropriate solvent (e.g., a mixture of 1,4-dioxane and water) generates compounds of Formula 12-1. Functionalization of compounds of Formula 12-1 using a number of conditions (e.g., including, but not limited to, electrophilic substitution or transition metal catalyzed reactions) affords compounds of Formula V. For example, alkylation of Formula 12-1 under appropriate conditions (e.g., using an appropriate alkylating agent, such as chloro(chloromethyl)dimethylsilane or methyl iodide, in the presence of a suitable base, such as 1,1,1,3,3,3-hexamethyldisilazane or potassium carbonate) in an appropriate solvent (e.g., CH₃CN or DMF) generates compounds of Formula V. Alternatively, transition metal (e.g., Cu) catalyzed cross-coupling reactions (including, but not limited to, Chan-Lam coupling) between compounds of Formula 12-1 and an appropriate coupling partner (e.g., methylboronic acid) affords compounds of Formula V.

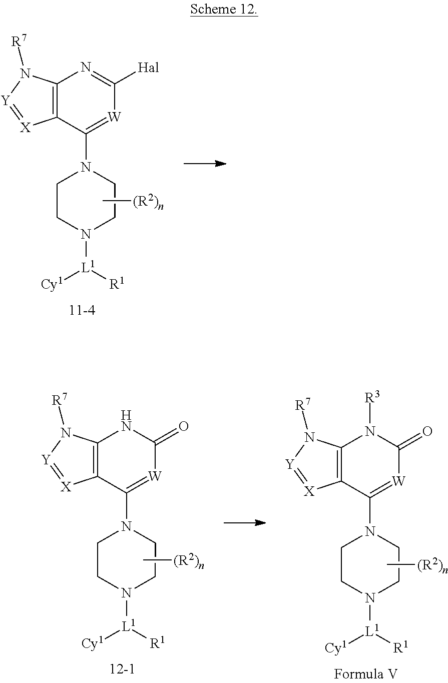

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature”, or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### PD-1/PD-L1 Inhibitors

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al., J. Clinical Oncology 2015, 1-9). Blocking the signaling of an inhibitory immune checkpoint, such as PD-1, has proven to be a promising and effective treatment modality.

Programmed Death-1 (“PD-1,” also known as “CD279”) is an approximately 31 kD type I membrane protein member of the extended CD28/CTLA-4 family of T-cell regulators that broadly negatively regulates immune responses (Ishida, Y. et al. (1992) EMBO J. 11:3887-3895; United States Patent Publication No. 2007/0202100; 2008/0311117; and 2009/00110667; U.S. Pat. Nos. 6,808,710; 7,101,550; 7,488,802; 7,635,757; and 7,722,868; PCT Publication No. WO 01/14557).

PD-1 is expressed on activated T-cells, B-cells, and monocytes (Agata, Y. et al. (1996) Int. Immunol. 8(5):765-772; Yamazaki, T. et al. (2002) J. Immunol. 169:5538-5545) and at low levels in natural killer (NK) T-cells (Nishimura, H. et al. (2000) J. Exp. Med. 191:891-898; Martin-Orozco, N. et al. (2007) Semin. Cancer Biol. 17(4):288-298).

The extracellular region of PD-1 consists of a single immunoglobulin (Ig)V domain with 23% identity to the equivalent domain in CTLA-4 (Martin-Orozco, N. et al. (2007) Semin. Cancer Biol. 17(4):288-298). The extracellular IgV domain is followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals (Ishida, Y. et al. (1992) EMBO J. 11:3887-3895; Blank, C. et al. (2006) Immunol. Immunother. 56(5):739-745).

PD-1 mediates its inhibition of the immune system by binding to B7-H1 and B7-DC (Flies, D. B. et al. (2007) J. Immunother. 30(3):251-260; U.S. Pat. Nos. 6,803,192; 7,794,710; United States Patent Application Publication Nos. 2005/0059051; 2009/0055944; 2009/0274666; 2009/0313687; PCT Publication Nos. WO 01/39722; WO 02/086083).

The amino acid sequence of the human PD-1 protein (Genbank Accession No. NP_005009) is:

(SEQ ID NO: 1)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-7 treatment (Iwai et al, PNAS 2002, 99(19):12293-7; Blank et al, Cancer Res 2004, 64(3):1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-7 production, as well as cell proliferation induced upon T cell receptor activation (Carter et al, Eur J Immunol 2002, 32(3):634-43; Freeman et al, J Exp Med 2000, 192(7):1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and AP1 pathways, and for cytokine production such as IL-2, IFN-7 and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al., Science 2001, 291:319-322). Using an LCMV model of chronic infection, it has been shown that PD-1/PD-L1 interaction inhibits activation, expansion and acquisition of effector functions of virus-specific CD8 T cells (Barber et al., Nature 2006, 439, 682-7). Together, these data support the development of a therapeutic approach to block the PD-1-mediated inhibitory signaling cascade in order to augment or “rescue” T cell response. Accordingly, there is a need for new methods of blocking PD-1/PD-L1 protein/protein interaction, and thereby treating cancer in a subject.

In some embodiments, the inhibitor of PD-1/PD-L1 is a compound selected from retifanlimab, nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IB1I308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054, and one or more of the PD-1/PD-L1 blocking agents described in U.S. Pat. Nos. 7,488,802, 7,943,743, 8,008,449, 8,168,757, 8,217,149, 10,166,221, 10,308,644, 10,618,916, 10,669,271, 10,793,565, 10,800,768, 10,806,785, 10,906,920, 11,124,511, 11,339,149, 11,401,279, 11,407,749, 11,414,433, 11,465,981, 11,535,615, 11,566,026, 11,572,366, 11,608,337, 11,613,536, 11,673,883, 11,718,605, 11,753,406, 11,760,756, 11,780,836, 11,787,793, or Pub. Nos. WO 2003/042402, WO 2008/156712, WO 2010/089411, WO 2010/036959, WO 2011/066342, WO 2011/159877, WO 2011/082400, WO 2011/161699, WO 2017/070089, WO 2017/087777, WO 2017/106634, WO 2017/112730, WO 2017/192961, WO 2017/205464, WO 2017/222976, WO 2018/013789, WO 2018/04478, WO 2018/119236, WO 2018/119266, WO 2018/119221, WO 2018/119286, WO 2018/119263, WO 2018/119224, WO 2019/191707, WO 2019/217821, WO 2021/030162, WO 2022/099018, WO 2021/096849, WO 2021/067217, WO 2022/099071, WO 2022/099075, WO 2022/133176, WO 2023/049831, and any combinations thereof. The disclosure of each of the preceding patents, applications, and publications is incorporated herein by reference in its entirety.

In some embodiments, the inhibitor of PD-1/PD-L1 is RMP1-14.

In some embodiments, the inhibitor of PD-1/PD-L1 is a humanized antibody.

In some embodiments, the inhibitor of PD-1/PD-L1 is pembrolizumab.

In some embodiments, the inhibitor of PD-1/PD-L1 is nivolumab.

In some embodiments, the inhibitor of PD-1/PD-L1 is atezolizumab.

In some embodiments, the inhibitor of PD-1/PD-L1 is an antibody or antigen-binding fragment thereof that binds to human PD-1. In some embodiments, the antibody or antigen-binding fragment thereof that binds to human PD-1 is a humanized antibody.

In some embodiments, the inhibitor of PD-1/PD-L1 is retifanlimab (i.e., MGA-012).

Retifanlimab is a humanized IgG4 monoclonal antibody that binds to human PD-1. See hPD-1 mAb 7(1.2) in U.S. Pat. No. 10,577,422, which is incorporated herein by reference in its entirety. The amino acid sequences of the mature retifanlimab heavy and light chains are shown below. Complementarity-determining regions (CDRs) 1, 2, and 3 of the variable heavy (VH) domain and the variable light (VL) domain are shown in that order from N to the C-terminus of the mature VL and VH sequences and are both underlined and bolded. An antibody consisting of the mature heavy chain (SEQ ID NO:2) and the mature light chain (SEQ ID NO:3) listed below is termed retifanlimab.

### Mature Retifanlimab Heavy Chain (HC)

(SEQ ID NO: 2)
QVQLVQSGAEVKKPGASVKVSCKASGYSFTSYWMNWVRQAPGQGLEWIG
VIHPSDSETWLDQKFKDRVTITVDKSTSTAYMELSSLRSEDTAVYYCAR
EHYGTSPFAYWGQGTLVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLG

### Mature Retifanlimab Light Chain (LC)

(SEQ ID NO: 3)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC

The variable heavy (VH) domain of retifanlimab has the following amino acid sequence:

(SEQ ID NO: 4)
QVQLVQSGAEVKKPGASVKVSCKASGYSFTSYWMNWVRQAPGQGLEWIG
VIHPSDSETWLDQKFKDRVTITVDKSTSTAYMELSSLRSEDTAVYYCAR
EHYGTSPFAYWGQGTLVTVSS

The variable light (VL) domain of retifanlimab has the following amino acid sequence:

(SEQ ID NO: 5)
EIVLTQSPATLSLSPGERATLSCRASESVDNYGMSFMNWFQQKPGQPPK
LLIHAASNQGSGVPSRFSGSGSGTDFTLTISSLEPEDFAVYFCQQSKEV
PYTFGGGTKVEIK

The amino acid sequences of the VH CDRs of retifanlimab are listed below:

VH CDR1:
(SEQ ID NO: 6)
SYWMN;
VH CDR2:
(SEQ ID NO: 7)
VIHPSDSETWLDQKFKD;
VH CDR3:
(SEQ ID NO: 8)
EHYGTSPFAY

The amino acid sequences of VL CDRs of retifanlimab are listed below:

VL CDR1:
(SEQ ID NO: 9)
RASESVDNYGMSFMNW;
VL CDR2:
(SEQ ID NO: 10)
AASNQGS;
and
VL CDR3:
(SEQ ID NO: 11)
QQSKEVPYT.

In some embodiments, the inhibitor of PD-1/PD-L1 is an antibody or antigen-binding fragment thereof that binds to human PD-1, wherein the antibody or antigen-binding fragment thereof comprises a variable heavy (VH) domain comprising VH complementarity determining region (CDR)1, VH CDR2, and VH CDR3, wherein:
the VH CDR1 comprises the amino acid sequence SYWMN (SEQ ID NO:6);
the VH CDR2 comprises the amino acid sequence VIIIPSDSETWLDQKFKD (SEQ ID NO:7); and
the VH CDR3 comprises the amino acid sequence EHYGTSPFAY (SEQ ID NO:8); and
wherein the antibody comprises a variable light (VL) domain comprising VL CDR1, VL CDR2, and VL CDR3, wherein:
the VL CDR1 comprises the amino acid sequence RASESVDNYGMSFMNW (SEQ ID NO:9);
the VL CDR2 comprises the amino acid sequence AASNQGS (SEQ ID NO:10); and
the VL CDR3 comprises the amino acid sequence QQSKEVPYT (SEQ ID NO:11).

Anti-PD-1 antibodies, such as retifanlimab, can be made, for example, by preparing and expressing synthetic genes that encode the recited amino acid sequences or by mutating human germline genes to provide a gene that encodes the recited amino acid sequences. Moreover, this antibody and other anti-PD-1 antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on Kabat numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-PD-1 antibodies can be in the form of full length antibodies, or in the form of low molecular weight forms (e.g., biologically active antibody fragments or minibodies) of the anti-PD-1 antibodies, e.g., Fab, Fab′, F(ab′)₂, Fv, Fd, dAb, scFv, and sc(Fv)2. Other anti-PD-1 antibodies encompassed by this disclosure include single domain antibody (sdAb) containing a single variable chain such as, VH or VL, or a biologically active fragment thereof. See, e.g., Moller et al., J. Biol. Chem., 285(49): 38348-38361 (2010); Harmsen et al., Appl. Microbiol. Biotechnol., 77(1):13-22 (2007); U.S. 2005/0079574 and Davies et al. (1996) Protein Eng., 9(6):531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Provided herein are compositions comprising a mixture of an anti-PD-1 antibody or antigen-binding fragment thereof and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-PD-1 antibody or antigen-binding fragment thereof comprising at least one deamidation site, wherein the pH of the composition is from about 5.0 to about 6.5, such that, e.g., at least about 90% of the anti-PD-1 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-PD-1 antibody or antigen-binding fragment thereof, means the presence of both the desired anti-PD-1 antibody or antigen-binding fragment thereof and one or more acidic variants thereof. The acidic variants may comprise predominantly deamidated anti-PD-1 antibody, with minor amounts of other acidic variant(s).

In some embodiments, the binding affinity (K_{D}), on-rate (K_{D} on) and/or off-rate (K_{D} Off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

In some embodiments, the retifanlimab is administered to the subject is a dosage of from about 250 mg to about to about 850 mg. In some embodiments, the retifanlimab is administered to the subject is a dosage of from about 375 mg to about to about 850 mg. In some embodiments, the retifanlimab is administered to the subject is a dosage of from about 450 mg to about to about 850 mg. In some embodiments, the retifanlimab is administered to the subject is a dosage of from about 500 mg to about to about 750 mg. In some embodiments, the retifanlimab is administered to the subject is a dosage of about 500 mg. In some embodiments, the retifanlimab is administered to the subject is a dosage of about 750 mg. In some embodiments, the retifanlimab is administered to the subject every four weeks. In some embodiments, the retifanlimab is administered to the subject by intravenous administration.

In some embodiments, the retifanlimab is administered to the subject at a dosage of 1 mg/kg Q2W.

In some embodiments, the retifanlimab is administered to the subject at a dosage of 3 mg/kg Q2W.

In some embodiments, the retifanlimab is administered to the subject at a dosage of 3 mg/kg Q4W.

In some embodiments, the retifanlimab is administered to the subject at a dosage of 10 mg/kg Q2W.

In some embodiments, the retifanlimab is administered to the subject at a dosage of 10 mg/kg Q4W.

In some embodiments, the retifanlimab is administered to the subject at a dosage of 200 mg Q3W.

In some embodiments, the retifanlimab is administered to the subject at a dosage of 250 mg Q3W.

In some embodiments, the retifanlimab is administered to the subject at a dosage of 375 mg Q3W.

In some embodiments, the retifanlimab is administered to the subject at a dosage of 500 mg Q4W.

In some embodiments, the retifanlimab is administered to the subject at a dosage of 750 mg Q4W.

In some embodiments, the retifanlimab is administered to the subject in a dosage of about 100 mg to about 1000 mg Q4W.

In some embodiments, the methods provided comprise administration of a first dosage of the inhibitor of PD-1/PD-L1, as defined herein, and a second dosage of the inhibitor of PD-1/PD-L1, wherein the second dosage is greater than the first dosage (i.e., the method comprises a dose escalation of the inhibitor of PD-1/PD-L1, such as retifanlimab).

In some embodiments, the inhibitor of PD-1/PD-L1 is selected from a compound as disclosed in WO 2018/119266 such as, e.g.,
(S)-1-((7-chloro-2-(2′-chloro-3′-(5-(((2-hydroxyethyl)amino)methyl)picolinamido)-2-methyl-[1,1′-biphenyl]-3-yl)benzo[d]oxazol-5-yl)methyl)piperidine-2-carboxylic acid, or a pharmaceutically acceptable salt thereof;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid, or a pharmaceutically acceptable salt thereof,
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof,
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof,
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid, or a pharmaceutically acceptable salt thereof;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)pyrrolidine-3-carboxylic acid, or a pharmaceutically acceptable salt thereof; and
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid, or a pharmaceutically acceptable salt thereof.

In some embodiments, the inhibitor of PD-1/PD-L1 is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof is also referred to herein as Compound Y. The synthesis and characterization of Compound Y is disclosed in Pub. Nos. WO 2018/119266 and WO 2022/099018, each of which are hereby incorporated by reference in their entirety.

Salt and solid forms of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, and pharmaceutically acceptable salts thereof, may be prepared. The synthesis and characterization of salt and solid forms of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid is described in U.S. Pat. No. 11,753,406 and Pub. No. WO 2021/030162, each of which are hereby incorporated by reference in their entirety.

In some embodiments, the inhibitor of PD-1/PD-L1 is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrobromic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid oxalic acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid hydrochloric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid L-tartaric acid salt;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid malonic acid salt; and
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid phosphoric acid salt.

Crystalline forms of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, and pharmaceutically acceptable salts thereof, may be prepared. The synthesis and characterization of salt and solid forms of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid is described in U.S. Pat. No. 11,760,756 and Pub. No. WO 2022/099075, each of which are hereby incorporated by reference in their entirety.

In some embodiments, the inhibitor of PD-1/PD-L1 is selected from:
a crystalline, tetrahydrofuran solvate of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid; and
an amorphous form of (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid.

In some embodiments, the inhibitor of PD-1/PD-L1 is selected from a compound disclosed in WO 2018/119224 such as, e.g.,
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid, or a pharmaceutically acceptable salt thereof,
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid, or a pharmaceutically acceptable salt thereof;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide, or a pharmaceutically acceptable salt thereof;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid, or a pharmaceutically acceptable salt thereof;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid, or a pharmaceutically acceptable salt thereof; and
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid, or a pharmaceutically acceptable salt thereof.

In some embodiments, the inhibitor of PD-1/PD-L1 is selected from a compound disclosed in WO 2019/191707 such as, e.g.,
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof,
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof,
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide, or a pharmaceutically acceptable salt thereof;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid, or a pharmaceutically acceptable salt thereof; and
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

In some embodiments, the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid (i.e., “Compound A”), or a pharmaceutically acceptable salt thereof.

The synthesis and characterization of (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid is disclosed in U.S. Pat. Nos. 10,669,271 and 11,124,511, and Pub. Nos. WO 2019/191707 and WO 2019/191707, each of which are hereby incorporated by reference in their entirety.

Salt and solid forms of (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, and pharmaceutically acceptable salts thereof, may be prepared. The synthesis and characterization of salt and solid forms of (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid is described in Pub. No. WO 2019/191707, which is hereby incorporated by reference in its entirety.

In some embodiments, the inhibitor of PD-1/PD-L1 is a crystalline form of (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments, the inhibitor of PD-1/PD-L1 is selected from a compound disclosed in WO 2019/217821 such as, e.g.,
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof;
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof;
4-(2-(2-((2,2′-dimethyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof; and
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments, the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof.

The synthesis and characterization of 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) is disclosed in U.S. Pat. Nos. 10,618,916, 10,906,920, 11,414,433, and Pub. Nos. WO2019/217821, each of which are hereby incorporated by reference in their entirety.

Salt and solid forms of 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), and pharmaceutically acceptable salts thereof, may be prepared. The synthesis and characterization of salt and solid forms of 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) is described in Pub. No. WO 2021/096849, which is hereby incorporated by reference in its entirety.

In some embodiments, the inhibitor of PD-1/PD-L1 is selected from:
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) di-hydrochloric acid salt;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) mono-hydrochloric acid salt; and
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) di-sodium salt.

In some embodiments, the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) di-hydrochloric acid salt.

In some embodiments, the inhibitor of PD-1/PD-L1 is crystalline 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid).

In some embodiments, the inhibitor of PD-1/PD-L1 is selected from a compound disclosed in WO 2021/067217 such as, e.g.,
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(pyrrolidin-1-ylmethyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((((1R,2S)-2-hydroxycyclopentyl)amino)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2S,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4S)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxyazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxy-3-methylazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid; and
(S)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments, the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments, the inhibitor of PD-1/PD-L1, or a pharmaceutically acceptable salt thereof, is administered to the subject in a dosage of from about 0.1 mg to about 1000 mg on a free base basis. In some embodiments, the inhibitor of PD-1/PD-L1, or a pharmaceutically acceptable salt thereof, is administered to the subject in a dosage of from about 1 mg to about 500 mg on a free base basis. In some embodiments, the inhibitor of PD-1/PD-L1, or a pharmaceutically acceptable salt thereof, is administered to the subject in a dosage of from about 5 mg to about 250 mg on a free base basis. In some embodiments, the inhibitor of PD-1/PD-L1, or a pharmaceutically acceptable salt thereof, is administered to the subject in a dosage of from about 10 mg to about 100 mg on a free base basis.

In some embodiments, the inhibitor of PD-1/PD-L1, or a pharmaceutically acceptable salt thereof, is administered to the subject in a dosage selected from about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, and about 1000 mg on a free base basis. In some embodiments, the inhibitor of PD-1/PD-L1, or a pharmaceutically acceptable salt thereof, is administered to the subject in a dosage ranging from about 0.1 mg to about 500 mg on a free base basis, or any dosage value there between. In some embodiments, the inhibitor of PD-1/PD-L1, or a pharmaceutically acceptable salt thereof, is administered to the subject in a dosage ranging from about 1 mg to about 100 mg on a free base basis, or any dosage value there between.

In some embodiments, the inhibitor of PD-1/PD-L1 is administered to the subject in a dosage of about 1 mg/kg to about 10 mg/kg. In some embodiments, the inhibitor of PD-1/PD-L1 is administered to the subject in a dosage of about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some embodiments, the inhibitor of PD-1/PD-L1 is administered to the subject in a dosage of about 200 mg to about 1000 mg. In some embodiments, the inhibitor of PD-1/PD-L1 is administered to the subject in a dosage of about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg or about 1000 mg.

In some embodiments, the inhibitor of PD-1/PD-L1 is administered to the subject once-daily, every other day, once-weekly or any time intervals between. In some embodiments, the inhibitor of PD-1/PD-L1 is administered to the subject once-daily. In some embodiments, the inhibitor of PD-1/PD-L1 is administered to the subject every other day. In some embodiments, the inhibitor of PD-1/PD-L1 is administered to the subject once-weekly.

In some embodiments, each of the dosages is administered as a single, once daily dosage. In some embodiments, each of the dosages is administered as a single, once daily oral dosage.

In some embodiments, the inhibitor of PD-1/PD-L1 is administered to the subject every two weeks, every three weeks or every four weeks. In some embodiments, the inhibitor of PD-1/PD-L1 is administered to the subject monthly or quarterly. In some embodiments, the inhibitor of PD-1/PD-L1 is administered to the subject by intravenous administration.

In some embodiments, the inhibitor of PD-1/PD-L1 is administered to the subject at a dosage of 1 mg/kg Q2W.

In some embodiments, the inhibitor of PD-1/PD-L1 is administered to the subject at a dosage of 3 mg/kg Q2W.

In some embodiments, the inhibitor of PD-1/PD-L1 is administered to the subject at a dosage of 3 mg/kg Q4W.

In some embodiments, the inhibitor of PD-1/PD-L1 is administered to the subject at a dosage of 10 mg/kg Q2W.

In some embodiments, the inhibitor of PD-1/PD-L1 is administered to the subject at a dosage of 10 mg/kg Q4W.

In some embodiments, the inhibitor of PD-1/PD-L1 is administered to the subject at a dosage of 200 mg Q3W.

In some embodiments, the inhibitor of PD-1/PD-L1 is administered to the subject at a dosage of 250 mg Q3W.

In some embodiments, the inhibitor of PD-1/PD-L1 is administered to the subject at a dosage of 375 mg Q3W.

In some embodiments, the inhibitor of PD-1/PD-L1 is administered to the subject at a dosage of 500 mg Q4W.

In some embodiments, the inhibitor of PD-1/PD-L1 is administered to the subject at a dosage of 750 mg Q4W.

### Methods of Use

In certain embodiments, the disclosure provides a method for treating a DGK-related disorder, or a PD-1/PD-L1 related disorder, or a combination thereof, in a patient in need thereof, comprising the step of administering to said patient a combination of the disclosure (e.g., a combination of a DGK inhibitor and a PD-1/PD-L1 inhibitor as described herein), or a pharmaceutically acceptable composition thereof.

In some embodiments, the present application provides a method of treating cancer in a subject, comprising administering to the subject:
(i) an inhibitor DGK provided herein; and
(ii) an inhibitor of PD-1/PD-L1 provided herein.

In some embodiments, the present application provides a method of treating cancer in a subject, comprising administering to the subject:
(i) an inhibitor DGK; and
(ii) an inhibitor of PD-1/PD-L1 provided herein,
   wherein the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine (i.e., “Compound 1”);
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
   or a pharmaceutically acceptable salt thereof.

In some embodiments of the methods provided herein, the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof.

In some embodiments of the methods provided herein, the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof.

In some embodiments of the methods provided herein, the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof.

In some embodiments of the methods provided herein, the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments of the methods provided herein, the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments of the methods provided herein, the DGK inhibitor is selected from:
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
   or a pharmaceutically acceptable salt thereof.

In some embodiments of the methods provided herein, the inhibitor of PD-1/PD-L1 is selected from:
(S)-1-((7-chloro-2-(2′-chloro-3′-(5-(((2-hydroxyethyl)amino)methyl)picolinamido)-2-methyl-[1,1′-biphenyl]-3-yl)benzo[d]oxazol-5-yl)methyl)piperidine-2-carboxylic acid;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid;
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)pyrrolidine-3-carboxylic acid;
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid;
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid;
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid;
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid);
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2,2′-dimethyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(pyrrolidin-1-ylmethyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((((1R,2S)-2-hydroxycyclopentyl)amino)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2S,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4S)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxyazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxy-3-methylazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid; and
(S)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein, the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments of the methods provided herein, the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof.

In some embodiments of the methods provided herein, the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments of the methods provided herein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
   or a pharmaceutically acceptable salt thereof; and
   (ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof; and
   (ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or
   a pharmaceutically acceptable salt thereof.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or
   a pharmaceutically acceptable salt thereof.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
   or a pharmaceutically acceptable salt thereof.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

In some embodiments of the methods provided herein, the inhibitor of PD-1/PD-L1 is selected from retifanlimab, nivolumab, pembrolizumab, atezolizumab, and RMP1-14.

In some embodiments of the methods provided herein, the inhibitor of PD-1/PD-L1 is selected from retifanlimab, nivolumab, and pembrolizumab.

In some embodiments of the methods provided herein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof, and
   (ii) the inhibitor of PD-1/PD-L1 is selected from retifanlimab, nivolumab, and pembrolizumab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is selected from retifanlimab, nivolumab, pembrolizumab, atezolizumab, and RMP1-14.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is selected from retifanlimab, nivolumab, and pembrolizumab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is retifanlimab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is nivolumab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is pembrolizumab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is atezolizumab.

In some embodiments of the methods provided herein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is RMP1-14.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is selected from retifanlimab, nivolumab, pembrolizumab, atezolizumab, and RMP1-14.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is selected from retifanlimab, nivolumab, and pembrolizumab.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is retifanlimab.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is nivolumab.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is pembrolizumab.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is atezolizumab.

In some embodiments of the methods provided herein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is RMP1-14.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is selected from retifanlimab, nivolumab, pembrolizumab, atezolizumab, and RMP1-14.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is selected from retifanlimab, nivolumab, and pembrolizumab.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is retifanlimab.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is nivolumab.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is pembrolizumab.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is atezolizumab.

In some embodiments of the methods provided herein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is RMP1-14.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is selected from retifanlimab, nivolumab, pembrolizumab, atezolizumab, and RMP1-14.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is retifanlimab.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is nivolumab.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is pembrolizumab.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is atezolizumab.

In some embodiments of the methods provided herein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is RMP1-14.

In some embodiments of the methods provided herein, the DGK inhibitor and the inhibitor of PD-1/PD-L1 are administered simultaneously. In some embodiments of the methods provided herein, the DGK inhibitor and the inhibitor of PD-1/PD-L1 are administered sequentially.

In some embodiments, the DGK- and/or PD-1/PD-L1-related disorder is a solid tumor. Example solid tumors include, but are not limited to, breast cancer, colorectal cancer, gastric cancer, and glioblastoma (see e.g., Cooke & Kazanietz, Sci. Signal, 2022, 15, eabo0264:1-26). Example cancers associated with alterations in DAG-regulating enzymes and effector include, but are not limited to, uveal melanoma, myelodysplastic syndrome (MDS), angiosarcoma, nodal peripheral T cell lymphoma, adult T-cell leukemia lymphoma (ATLL), cutaneous T-cell lymphoma (CTCL)/Sezary syndrome, chronic lymphocytic leukemia (CLL), breast cancer, gastric cancer, colorectal cancer, oral squamous cell carcinoma (SCC), esophageal SCC, chronic myeloid leukemia (CML), colon cancer, prostate cancer, hepatocellular carcinoma (HCC), blue nevi, NK/T cell lymphoma, glioma, ovarian cancer, liver cancer, melanoma, heptacarcinoma, ostersarcoma, chordiod glioma, pigmented epithelioid melanocytoma, papillary glioneuronal tumor, fibrous histiocytoma, pituitary tumor, thyroid cancer, head and neck SCC, lung cancer, pediatric T-cell acute lymphoblastic leukemia (T-ALL), endometrial cancer, angiolipoma, salivary gland cancer, acute myeloid leukemia (AML), Epstein-Barr virus-associated (EBV)-associated B cell lymphoma, diffuse large B cell lymphoma (DLBCL), and cervical cancer (see e.g., Cooke & Kazanietz, Sci. Signal, 2022, 15, eabo0264:1-26).

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (MS), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor Tlymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as Tprolymphocytic leukemia (T-PLL), including of the small cell and cerebriform cell type; large granular lymphocyte leukemia (LGL) of the T-cell type; aid T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable cancers, refractory cancers (e.g., cancers refractory to previous immunotherapy, e.g., with a blocking PD-1 antibody), and/or recurrent cancers.

In some embodiments, the tumor comprises one or more genetic features selected from high microsatellite instability (MSI-H), mismatch repair deficient (MMRd), and high tumor mutational burden (TMB-H), or any combination thereof.

In some embodiments, the tumor comprises high microsatellite instability (MSI-H), mismatch repair deficient (MMRd), high tumor mutational burden (TMB-H), or mismatch repair deficient (MMRd) and high tumor mutational burden (TMB-H).

In some embodiments, the tumor is identified or has been identified as comprising one or more genetic features selected from high microsatellite instability (MSI-H), mismatch repair deficient (MMRd), and high tumor mutational burden (TMB-H), or any combination thereof.

In some embodiments, the tumor is identified or has been identified comprising high microsatellite instability (MSI-H), mismatch repair deficient (MMRd), high tumor mutational burden (TMB-H), or mismatch repair deficient (MMRd) and high tumor mutational burden (TMB-H).

In some embodiments, the tumor is a tumor with high microsatellite instability (MSI-H). In some embodiments, the tumor is mismatch repair deficient (MMRd). In some embodiments, the tumor is a tumor with high tumor mutational burden (TMB-H). In some embodiments, the tumor is mismatch repair deficient (MMRd) and comprises high tumor mutational burden (TMB-H).

In some embodiments, the cancer is amenable to (e.g., treatable with; responds positively to treatment with; and the like) T cell mediated immunotherapy.

In some embodiments, the cancer is selected from lung cancer, bladder cancer, urothelial cancer, esophageal cancer, stomach cancer, mesothelioma, liver cancer, diffuse large B cell lymphoma, kidney cancer, head and neck cancer, cholangiocarcinoma, cervical cancer, endocervical cancer, melanoma, merkel cell carcinoma (MCC), cutaneous squamous cell carcinoma (CSCC), melanoma, MSI high tumors, ICI sensitive tumors, and viral infection related cancers such as HPV-associated anal cancer, vaginal cancer, vulvar cancer, cervical cancer and oropharyngeal cancer.

In some embodiments, the cancer is selected from lung cancer, bladder cancer, urothelial cancer, esophageal cancer, stomach cancer, mesothelioma, liver cancer, diffuse large B cell lymphoma, kidney cancer, head and neck cancer, cholangiocarcinoma, cervical cancer, endocervical cancer, and melanoma.

In some embodiments, the cancer is selected from non-small cell lung cancer (lung squamous cell carcinoma (LUSC), lung adenocarcinoma (LUAD)), bladder urothelial carcinoma, esophageal carcinoma, stomach adenocarcinoma, mesothelioma, liver hepatocellular carcinoma, diffuse large B cell lymphoma (DLBCL), kidney renal clear cell carcinoma, head and neck squamous cell carcinoma, cholangiocarcinoma, cervical squamous cell carcinoma, endocervical adenocarcinoma, and metastatic melanoma.

In some embodiments, the cancer is a myelodysplastic syndrome. As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1-A) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1-A
2008 WHO Classification for De Novo Myelodysplastic Syndrome

| Subtype | Blood | Bone Marrow |
|---|---|---|
| Refractory cytopenia | Single or | Dysplasia in ≥10% of |
| with unilineage | Bicytopenia | 1 cell line, <5% blasts |
| dysplasia (RCUD) | | |
| Refractory anemia | Anemia, no blasts | ≥15% of erythroid |
| with ring sideroblasts | | precursors w/ring |
| (RARS) | | sideroblasts, erythroid |
| | | dysplasia only, <5% blasts |
| Refractory cytopenia | Cytopenia(s), <1 × | Dysplasia in ≥10% of |
| with multilineage | 10⁹/L monocytes | cells in ≥2 hematopoietic |
| dysplasia | | lineages, ±15% ring |
| | | sideroblasts, <5% blasts |
| Refractory anemia | Cytopenia(s), ≤2% | Unilineage or multilineage |
| with excess blasts-1 | to 4% | dysplasia, No Auer rods, |
| (RAEB-1) | blasts, <1 × 10⁹/L | 5% to 9% blasts |
| | monocytes | |
| Refractory anemia | Cytopenia(s), ≤5% | Unilineage or multilineage |
| with excess blasts-2 | to 19% | dysplasia, ± Auer rods, |
| (RAEB-2) | blasts, <1 × 10⁹/L | 10% to 19% blasts |
| | monocytes | |
| Myelodysplastic | Cytopenias | Unilineage or no dysplasia |
| syndrome, | | but characteristic MDS |
| unclassified (MDS-U) | | cytogenetics, <5% blasts |
| MDS associated with | Anemia, platelets | Unilineage erythroid. |
| isolated del(5q) | normal or increased | Isolated del(5q), <5% blasts |

In some embodiments, the myelodysplastic syndrome is refractory cytopenia with unilineage dysplasia (RCUID).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the combination of the disclosure can be useful in the treatment of myeloproliferative disorder/myelodysplastic overlap syndrome (MPD/MDS overlap syndrome).

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor.

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor; wherein the compound of Formula I, or a pharmaceutically acceptable salt thereof, is administered as a monotherapy.

In some embodiments, the present application further provides a method of treating a cancer in a subject, comprising administering to the subject the compound of Formula I, or a pharmaceutically acceptable salt thereof, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H); wherein the compound of Formula I, or a pharmaceutically acceptable salt thereof, is administered as a monotherapy.

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor; wherein the compound of Formula I, or a pharmaceutically acceptable salt thereof, is administered as a monotherapy, and wherein the compound of Formula I is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor; wherein the compound of Formula I, or a pharmaceutically acceptable salt thereof, is administered as a monotherapy, and wherein the compound of Formula I is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor; wherein the compound of Formula I, or a pharmaceutically acceptable salt thereof, is administered as a monotherapy, and wherein the compound of Formula I is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is an advanced cancer, metastatic cancer, solid tumor, advanced solid tumor, hematological tumor, cancers that are refractory to checkpoint inhibitors (or checkpoint antagonists), or cancers that have progressed after treatment with a checkpoint inhibitor; wherein the compound of Formula I, or a pharmaceutically acceptable salt thereof, is administered as a monotherapy, and wherein the compound of Formula I is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H); wherein the compound of Formula I, or a pharmaceutically acceptable salt thereof, is administered as a monotherapy, and wherein the compound of Formula I is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
   or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H); wherein the compound of Formula I, or a pharmaceutically acceptable salt thereof, is administered as a monotherapy, and wherein the compound of Formula I is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H); wherein the compound of Formula I, or a pharmaceutically acceptable salt thereof, is administered as a monotherapy, and wherein the compound of Formula I is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, the present application further provides a method of treating a cancer in a subject, comprising administering to the subject the compound of Formula I, or a pharmaceutically acceptable salt thereof, wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H); wherein the compound of Formula I, or a pharmaceutically acceptable salt thereof, is administered as a monotherapy, and wherein the compound of Formula I is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

In some embodiments, provided herein is a method of increasing survival or progression-free survival in a patient, comprising administering a combination provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the combination until the earlier of death or progression of the disease. Progression of the disease can be defined by RECIST v. 1.1 (Response Evaluation Criteria in Solid Tumors), as assessed by an independent centralized radiological review committee. In some embodiments, administering of the combination results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the combination results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the combination results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a combination described herein, for use in any of the methods described herein.

The present disclosure further provides use of a combination described herein, for the preparation of a medicament for use in any of the methods described herein.

As used herein, “about” when referring to a measurable value such as an amount, a dosage, a temporal duration, and the like, is meant to encompass variations of 10%. In certain embodiments, “about” can include variations of 5%, +1%, or ±0.1% from the specified value and any variations there between, as such variations are appropriate to perform the disclosed methods.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a DGK with a DGK inhibitor described herein includes the administration of a DGK inhibitor described herein to an individual or patient, such as a human, having a DGK, as well as, for example, introducing a DGK inhibitor described herein into a sample containing a cellular or purified preparation containing the DGK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound (e.g., a DGK inhibitor and/or PD-1/PD-L1 inhibitor described herein) or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science andPractice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, “QD” is taken to mean a dosage administered to the subject once-daily. “QOD” is taken to mean a dosage administered to the subject once, every other day. “QW” is taken to mean a dosage administered to the subject once-weekly. “Q2W” is taken to mean a dosage administered to the subject once, every other week. “Q3W” is taken to mean a dosage administered to the subject once, every three weeks. “Q4W” is taken to mean a dosage administered to the subject once, every four weeks.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the combinations of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Pharmaceutical Formulations

When employed as pharmaceuticals, the combination of the present disclosure can be administered in the form of one or more pharmaceutical compositions. Thus the present disclosure provides one or more compositions comprising a compound described herein (e.g., a DGK inhibitor and/or a PD-1/PD-L1 inhibitor), or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the one or more compounds of the combinations disclosed herein, or pharmaceutically acceptable salts thereof, in combination with one or more pharmaceutically acceptable carriers or excipients. In some embodiments, the composition is suitable for topical administration. In making the compositions of the disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The DGK inhibitors and/or PD-1/PD-L1 inhibitors of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one DGK inhibitor and/or PD-1/PD-L1 inhibitor described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one DGK inhibitor and/or PD-1/PD-L1 inhibitor described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™) In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compounds of the combinations provided herein may be effective over a wide dosage range and are generally administered in a therapeutically effective amount. It will be understood, however, that the amount of the compounds actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of compounds of the combinations of the present disclosure can vary according to, e.g., the particular use for which the treatment is made, the manner of administration of the compound, the health and condition of the patient, and the judgment of the prescribing physician. The proportion or concentration of a compound of the combinations provided herein in a pharmaceutical composition can vary depending upon a number of factors including dosage, chemical characteristics (e.g., hydrophobicity), and the route of administration. For example, the compounds of combinations provided herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compounds selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt. % of a compound provided herein. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The combinations of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.). For example, labeled DGK inhibitors of the disclosure would be useful not only in imaging techniques but also in assays, both in vitro and in vivo, for localizing and quantitating DGK in tissue samples, including human, and for identifying DGK inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes DGK assays that contain such labeled or substituted DGK inhibitors.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms to allow the compound to be deuterated (e.g., one or more hydrogen atoms of a C₁₋₆ alkyl group of Formula I can be optionally substituted with deuterium atoms, such as —CD₃ being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, the compound provided herein (e.g., a compound of any of Formulas I-V), or a pharmaceutically acceptable salt thereof, comprises at least one deuterium atom.

In some embodiments, the compound provided herein (e.g., a compound of any of Formulas I-V), or a pharmaceutically acceptable salt thereof, comprises two or more deuterium atoms.

In some embodiments, the compound provided herein (e.g., a compound of any of Formulas I-V), or a pharmaceutically acceptable salt thereof, comprises three or more deuterium atoms.

In some embodiments, for a compound provided herein (e.g., a compound of any of Formulas I-V), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro DGK labeling and competition assays, DGK inhibitors that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind DGK by monitoring its concentration variation when contacting with DGK, through tracking of the labeling. For example, a test compound (labeled) can be evaluated for its ability to reduce binding of another compound which is known to bind to DGK (i.e., standard compound). Accordingly, the ability of a test compound to compete with the standard compound for binding to DGK directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of DGK- and/or PD-1/PD-L1-associated diseases or disorders as described herein, which include one or more containers containing a pharmaceutical composition comprising a therapeutically effective amount of (i) DGK inhibitor; and (ii) a PD-1/PD-L1 inhibitor of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)). The compounds separated were typically subjected to analytical liquid chromatography mass spectrometry (LCMS) for purity analysis under the following conditions: Instrument; Agilent 1100 series, LC/MSD, Column: Waters Sunfire™ C₁₈ 5 μm, 2.1×50 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm, 19×100 mm, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.
pH=10 purifications: Waters XBridge™ C₁₈ 5 μm, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (see e.g. “Preparative LCMS Purification: Improved Compound Specific Method Optimization”, K. Blom, B. Glass, R. Sparks, A. Combs, J. Comb. Chem., 6, 874-883 (2004)). For purifications using a 30×100 mm column, the flow rate was 60 mL/minute.

### Intermediate 1. (S)-2,6-Dichloro-9-((tetrahydrofuran-2-yl)methyl)-9H-purine

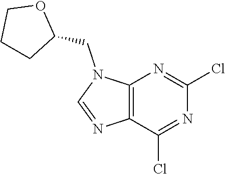 [SMILES: Clc1nc(Cl)c2ncn(C[C@@H]3CCCO3)c2n1]

To a mixture of 2,6-dichloro-9H-purine (27 g, 143 mmol), (S)-(tetrahydrofuran-2-yl)methanol (36.5 g, 357 mmol, BLD Pharmatech BD48351), and triphenylphosphine (94 g, 357 mmol) in THE (714 mL) was added diisopropyl azodicarboxylate (70.3 mL, 357 mmol, Aldrich 225541) and the reaction mixture was stirred at rt for 2 h. Additional (S)-(tetrahydrofuran-2-yl)methanol (1.46 g, 14.3 mmol), triphenylphosphine (3.75 g, 14.3 mmol), and diisopropyl azodicarboxylate (2.82 mL, 14.3 mmol) was added and the reaction mixture was stirred at rt for 1 h. Calcium bromide (140 g, 703 mmol) was added and the reaction mixture was stirred at rt overnight. The mixture was filtered to remove undissolved solids and the filter cake was washed with EtOAc. The filtrate was concentrated in vacuo and the crude residue was purified by flash column chromatography (EtOAc/hexanes). Fractions containing the desired product were combined and concentrated, and the material obtained was triturated with cold Et₂O to afford the desired product (12.5 g, 32% yield) as a white solid. LC-MS calculated for C₁₀H₁₁Cl₂N₄O (M+H)⁺: m/z=273.0. found 273.0.

### Intermediate 2. tert-Butyl (2S,5R)-4-(3,3-difluorocyclobutane-1-carbonyl)-2,5-dimethylpiperazine-1-carboxylate

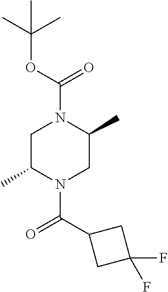 [SMILES: C[C@@H]1CN(C(=O)OC(C)(C)C)[C@@H](C)CN1C(=O)C1CC(F)(F)C1]

A mixture of tert-butyl (2S,5R)-2,5-dimethylpiperazine-1-carboxylate (6.00 g, 28.0 mmol, Combi-Blocks OR-8588) and 3,3-difluorocyclobutane-1-carboxylic acid (4.19 g, 30.8 mmol, Astatech 84107) in MeCN (25 mL) was treated with N,N-diisopropylethylamine (14.7 mL, 84.0 mmol) and HATU (11.2 g, 29.4 mmol, Combi-Blocks OR-0618) and stirred at rt for 30 min. The solvent was removed in vacuo and the residue was diluted with EtOAc and water. The layers were separated and the aqueous layer was extracted with EtOAc. The combined organic layers were washed with brine, dried over MgSO₄, concentrated in vacuo, and purified by flash column chromatography (120 g SiO₂, EtOAc/hexanes) to give the title compound (8.90 g, 96% yield) as a white solid. LC-MS calculated for C₁₂H₁₉F₂N₂O₃(M-C₄H₈+H)+: m/z=277.1. found 277.1.

### Intermediate 3. (2R,5S)-1-((3,3-Difluorocyclobutyl)(4-(trifluoromethyl)phenyl)methyl)-2,5-dimethylpiperazine hydrochloride

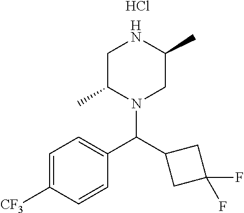 [SMILES: Cl ; C[C@@H]1CN[C@@H](C)CN1C(c1ccc(C(F)(F)F)cc1)C1CC(F)(F)C1]

### Step 1: (4-(Trifluoromethyl)phenyl)magnesium chloride lithium chloride (1.1 M in THF)

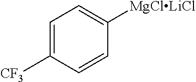

A 1.3 M solution of isopropylmagnesium chloride lithium chloride complex in THE (5.78 mL, 7.52 mmol, Aldrich 656984) was cooled to −78° C. before 1-bromo-4-(trifluoromethyl)benzene (1.14 mL, 8.27 mmol, Aldrich 152692) was added dropwise and the reaction mixture was stirred at −78° C. for 5 min. The reaction mixture was warmed to rt and stirred for an additional 4 h. The mixture obtained was used directly in the next step.

### Step 2: tert-Butyl (2S,5R)-4-((3,3-difluorocyclobutyl)(4-(trifluoromethyl)phenyl)methyl)-2,5-dimethylpiperazine-1-carboxylate

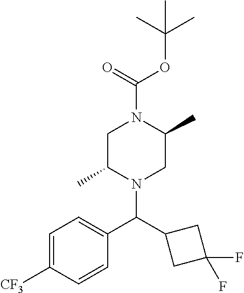 [SMILES: C[C@@H]1CN(C(=O)OC(C)(C)C)[C@@H](C)CN1C(c1ccc(C(F)(F)F)cc1)C1CC(F)(F)C1]

A mixture of tert-butyl (2S,5R)-4-(3,3-difluorocyclobutane-1-carbonyl)-2,5-dimethylpiperazine-1-carboxylate (Intermediate 2, 2.00 g, 6.02 mmol) and chlorocarbonylbis(triphenylphosphine)iridium(I) (0.469 g, 0.602 mmol, Strem 77-0300) in CH₂Cl₂ (10 mL) was treated with 1,1,3,3-tetramethyldisiloxane (2.13 mL, 12.0 mmol, Aldrich 235733) and stirred at rt for 15 min. Immediate gas evolution was observed, and the yellow color of the catalyst became bleached over the course of 15 min. The reaction was cooled to −78° C. and stirred for 5 min before (4-(trifluoromethyl)phenyl)magnesium chloride lithium chloride (Step 1, 6.92 mL, 1.1 M in THF, 7.5 mmol) was added dropwise and the reaction mixture was stirred for an additional 5 min. The reaction mixture was warmed to 0° C. and stirred for 30 min. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated in vacuo to afford the desired product as a mixture of diastereomers. The crude material obtained was used directly without further purification. LC-MS calculated for C₂₃H₃₂F₅N₂O₂(M+H)⁺: m/z=463.2. found 463.2.

### Step 3: (2R,5S)-1-((3,3-Difluorocyclobutyl)(4-(trifluoromethyl)phenyl)methyl)-2,5-dimethylpiperazine hydrochloride

A mixture of tert-butyl (2S,5R)-4-((3,3-difluorocyclobutyl)(4-(trifluoromethyl)phenyl)methyl)-2,5-dimethylpiperazine-1-carboxylate (Step 2) in THE (10 mL) was treated with HCl (4 M in 1,4-dioxane, 10 mL, 40 mmol, Oakwood 094030) and stirred at 60° C. for 1 h. After cooling to rt, the mixture was diluted with diethyl ether and the resulting precipitate was collected by filtration, washed with diethyl ether, and dried under vacuum to afford the desired product (1.50 g, 69% yield over two steps) as a mixture of diastereomers in the form of a white solid. LC-MS calculated for C₁₈H₂₄F₅N₂ (M+H)+: m/z=363.2. found 363.2.

### Intermediate 5. (S)-2,6-Dichloro-8-methyl-9-((tetrahydrofuran-2-yl)methyl)-9H-purine

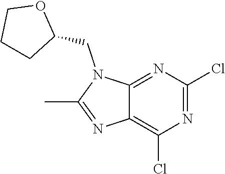 [SMILES: Cc1nc2c(Cl)nc(Cl)nc2n1C[C@@H]1CCCO1]

To a mixture of 2,6-dichloro-8-methylpurine (10.0 g, 49.3 mmol, PharmaBlock PB02898), (S)-(tetrahydrofuran-2-yl)methanol (5.53 g, 54.2 mmol, BLD Pharmatech BD48351), and triphenylphosphine, polymer-bound (100-200 mesh, extent of labeling: ˜1.6 mmol/g loading, Aldrich 93094, 62 g, 99 mmol) in THE (500 mL) was added diisopropyl azodicarboxylate (19.2 mL, 98.7 mmol, Aldrich 225541) and the reaction mixture was stirred at rt for 2 h. The mixture was filtered over Celite and the filtrate was concentrated in vacuo. The crude residue was purified by flash column chromatography (330 g SiO₂, CH₂Cl₂/EtOAc) to afford the desired product (6.8 g, 48% yield) as a white solid. LC-MS calculated for C₁₁H₁₃Cl₂N₄O (M+H)⁺: m/z=287.0. found 287.0.

### Intermediate 9. tert-Butyl (2S,5R)-2,5-dimethyl-4-(3-methylbutanoyl)piperazine-1-carboxylate

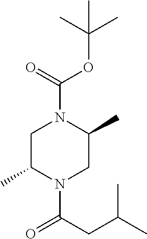 [SMILES: CC(C)CC(=O)N1C[C@H](C)N(C(=O)OC(C)(C)C)C[C@H]1C]

A mixture of tert-butyl (2S,5R)-2,5-dimethylpiperazine-1-carboxylate (2.14 g, 10.0 mmol, Combi-Blocks OR-8588) and N,N-diisopropylethylamine (3.49 mL, 20.00 mmol) in CH₂Cl₂ (33.3 mL) was cooled to 0° C. and isovaleryl chloride (1.463 mL, 12.00 mmol, Aldrich 157422) was added dropwise. The mixture was warmed to room temperature and stirred 30 minutes. Saturated aqueous NaHCO₃ (50 mL) was added and the mixture was stirred vigorously for 15 minutes. The layers were separated and the organic layer was washed with 1 M HCl (50 mL) and brine (50 mL), dried over MgSO₄, and concentrated in vacuo. The title compound (2.92 g, 98% yield) was obtained as a light yellow solid. LC-MS calculated for C₁₆H₃₁N₂O₃ (M+H)⁺: m/z=299.2. found 299.3.

### Intermediate 10. (2R,5S)-1-(1-(4-Chlorophenyl)-3-methylbutyl)-2,5-dimethylpiperazine hydrochloride

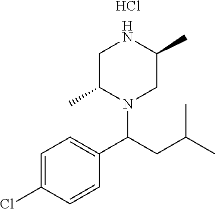 [SMILES: Cl ; CC(C)CC(c1ccc(Cl)cc1)N1C[C@H](C)NC[C@H]1C]

### Step 1: tert-Butyl (2S,5R)-4-(1-(4-chlorophenyl)-3-methylbutyl)-2,5-dimethylpiperazine-1-carboxylate

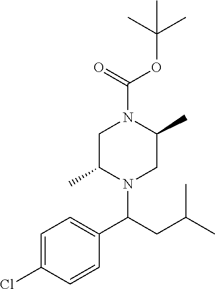 [SMILES: CC(C)CC(c1ccc(Cl)cc1)N1C[C@H](C)N(C(=O)OC(C)(C)C)C[C@H]1C]

A mixture of tert-butyl (2S,5R)-2,5-dimethyl-4-(3-methylbutanoyl)piperazine-1-carboxylate (Intermediate 9, 1.50 g, 5.03 mmol) and Ir(CO)Cl(PPh₃)₂(0.118 g, 0.151 mmol, Strem 77-0300) in CH₂Cl₂ (50 mL) was treated with 1,1,3,3-tetramethyldisiloxane (1.78 mL, 10.1 mmol, Aldrich 235733) and stirred 15 minutes at room temperature. After 15 minutes additional Ir(CO)Cl(PPh₃)₂(0.118 g, 0.151 mmol) and 1,1,3,3-tetramethyldisiloxane (0.89 mL, 5.05 mmol) were added and stirring was continued at room temperature for 15 minutes. The reaction was then cooled to −78° C. and stirred 5 minutes before (4-chlorophenyl)magnesium bromide (1.0 M in diethyl ether, 6.28 mL, 6.28 mmol, Aldrich 262188) was added dropwise. The reaction was stirred an additional 5 minutes, warmed to 0° C. and stirred 30 min. The reaction was quenched with sat. aqueous NH₄Cl. The layers were separated and the aqueous layer was extracted with CH₂Cl₂. The combined organic layers were dried over MgSO₄ and concentrated in vacuo. The material was purified by flash column chromatography (120 g SiO₂, EtOAc/hexanes) to give the title compound (1.74 g, 88% yield) as a single stereoisomer. LC-MS calculated for C₂₂H₃₆ClN₂O₂(M+H)⁺: m/z=395.3. found 395.2.

### Step 2: (2R,5S)-1-(1-(4-chlorophenyl)-3-methylbutyl)-2,5-dimethylpiperazine hydrochloride

A mixture of tert-butyl (2S,5R)-4-(1-(4-chlorophenyl)-3-methylbutyl)-2,5-dimethylpiperazine-1-carboxylate (1.74 g, 4.41 mmol) in THE (11.0 mL) was treated with HCl (4 M in dioxane, 11.1 mL, 44.2 mmol, Oakwood 094030). The mixture was stirred at stirred at 60° C. for 1 h. The mixture was cooled to room temperature, concentrated in vacuo to approximately half volume, diluted with diethyl ether (10 mL) and hexanes (5 mL), and the precipitate was collected by filtration (washing with 2:1 diethyl ether-hexanes) to give the title compound (1.29 g, 88% yield) as a white solid. LC-MS calculated for C₁₇H₂₈ClN₂ (M+H)⁺: m/z=295.2. found 295.2.

### Intermediate 12. (2R,5S)-1-(1-(4-Chloro-3-fluorophenyl)-3-methylbutyl)-2,5-dimethylpiperazine hydrochloride

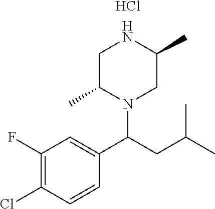 [SMILES: Cl ; CC(C)CC(c1ccc(Cl)c(F)c1)N1C[C@H](C)NC[C@H]1C]

### Step 1: (4-Chloro-3-fluorophenyl)magnesium chloride lithium chloride (0.5 M in THF)

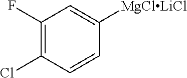

Isopropylmagnesium chloride lithium chloride complex (1.3 M in THF, 4.04 mL, 5.25 mmol, Aldrich 656984) was added dropwise to a solution of 1-chloro-2-fluoro-4-iodobenzene (1.28 g, 5.00 mmol, Oakwood 018374) in THE (10 mL total volume) at −20° C. and the reaction was stirred at this temperature for 30 min. The turbid mixture obtained was used directly in the next step.

### Intermediate 15. tert-Butyl (2S,5R)-4-((S)-2,2-difluorocyclopropane-1-carbonyl)-2,5-dimethylpiperazine-1-carboxylate

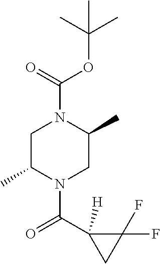 [SMILES: C[C@@H]1CN(C(=O)OC(C)(C)C)[C@@H](C)CN1C(=O)[C@@H]1CC1(F)F]

A mixture of tert-butyl (2S,5R)-2,5-dimethylpiperazine-1-carboxylate (2.14 g, 10.0 mmol Combi-Blocks OR-8588) and (S)-2,2-difluorocyclopropane-1-carboxylic acid (1.22 g, 10.00 mmol, AstaTech P15788) in MeCN (33 mL) was treated with N,N-diisopropylethylamine (3.49 mL, 20.0 mmol) and HATU (3.99 g, 10.5 mmol, Combi-Blocks OR-0618) and stirred at rt overnight. The solvent was removed in vacuo and the crude residue was diluted with EtOAc and water. The layers were separated and the aqueous layer was extracted with EtOAc. The combined organic layers were washed with brine, dried over MgSO₄, concentrated in vacuo, and purified by flash column chromatography (40 g SiO₂, EtOAc/hexanes) to give the title compound (2.82 g, 84% yield) as a white solid. LC-MS calculated for C₁₁H₁₇F₂N₂O₃(M-C₄H₈+H)⁺: m/z=263.1. found 263.2.

### Intermediate 16. (2R,5S)-1-((4-Chlorophenyl)((S)-2,2-difluorocyclopropyl)methyl)-2,5-dimethylpiperazine hydrochloride

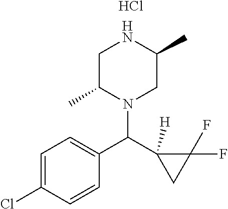 [SMILES: Cl ; C[C@@H]1CN[C@@H](C)CN1C(c1ccc(Cl)cc1)[C@@H]1CC1(F)F]

The title compound was prepared according to the procedures described for Intermediate 10, with tert-butyl (2S,5R)-4-((S)-2,2-difluorocyclopropane-1-carbonyl)-2,5-dimethylpiperazine-1-carboxylate (Intermediate 15) replacing tert-butyl (2S,5R)-2,5-dimethyl-4-(3-methylbutanoyl)piperazine-1-carboxylate. The title compound was isolated as a single stereoisomer. LC-MS calculated for C₁₆H₂₂ClF₂N₂(M+H)⁺: m/z=315.1. found 315.2.

### Intermediate 18. tert-Butyl (2S,5R)-4-((R)-2,2-difluorocyclopropane-1-carbonyl)-2,5-dimethylpiperazine-1-carboxylate

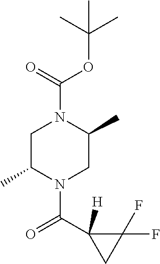 [SMILES: C[C@@H]1CN(C(=O)OC(C)(C)C)[C@@H](C)CN1C(=O)[C@H]1CC1(F)F]

The title compound was prepared according to the procedure described for Intermediate 15, with (R)-2,2-difluorocyclopropane-1-carboxylic acid (AstaTech P17160) replacing (S)-2,2-difluorocyclopropane-1-carboxylic acid. LC-MS calculated for C₁₁H₁₇F₂N₂O₃(M-C₄H₈+H)⁺: m/z=263.1. found 263.2.

### Intermediate 19. tert-Butyl (2S,5R)-4-((4-chlorophenyl)((R)-2,2-difluorocyclopropyl)methyl)-2,5-dimethylpiperazine-1-carboxylate

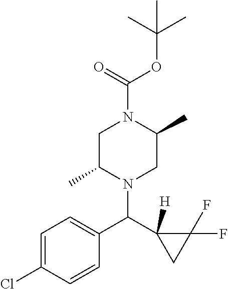 [SMILES: C[C@@H]1CN(C(=O)OC(C)(C)C)[C@@H](C)CN1C(c1ccc(Cl)cc1)[C@H]1CC1(F)F]

The title compound was prepared according to the procedure described in Step 1 for Intermediate 10, with tert-butyl (2S,5R)-4-((R)-2,2-difluorocyclopropane-1-carbonyl)-2,5-dimethylpiperazine-1-carboxylate (Intermediate 18) replacing tert-butyl (2S,5R)-2,5-dimethyl-4-(3-methylbutanoyl)piperazine-1-carboxylate. The title compound was isolated as a single stereoisomer. LC-MS calculated for C₂₁H₃₀ClF₂N₂O₂ (M+H)⁺: m/z=415.2. found 415.2.

### Intermediate 39. (2R,5S)-1-((4-Chlorophenyl)(3,3-difluorocyclobutyl)methyl)-2,5-dimethylpiperazine hydrochloride

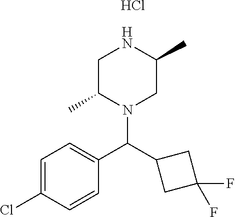 [SMILES: Cl ; C[C@@H]1CN[C@@H](C)CN1C(c1ccc(Cl)cc1)C1CC(F)(F)C1]

The title compound was prepared according to the procedures described in Intermediate 3, with 1-bromo-4-chlorobenzene replacing 1-bromo-4-(trifluoromethyl)benzene in Step 1. LC-MS calculated for C₁₇H₂₄ClF₂N₂(M+H)⁺: m/z=329.2. found 329.1.

### Intermediate 41. tert-Butyl (2S,5R)-5-ethyl-2-methylpiperazine-1-carboxylate

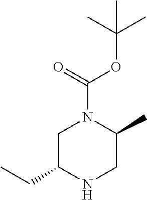 [SMILES: CC[C@@H]1CN(C(=O)OC(C)(C)C)[C@@H](C)CN1]

### Step 1: Methyl (R)-2-(benzylamino)butanoate

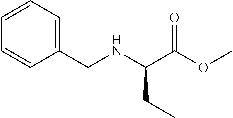

To a stirred solution of methyl (R)-2-aminobutanoate hydrochloride (30.0 g, 195 mmol, Combi-Blocks QA-7768) in CH₂Cl₂ (500 mL) was added benzaldehyde (20.7 g, 195 mmol) and the reaction mixture was stirred at rt for 6 h. The reaction mixture was cooled to 0° C. in an ice-bath before sodium triacetoxyborohydride (20.7 g, 98 mmol) was added portionwise over 20 min. The ice-bath was removed and the reaction mixture was stirred at ambient temperature overnight. The mixture was transferred to a separatory funnel and extracted with 1 M aqueous HCl (3×300 mL). The combined aqueous layers were made basic with solid KOH (pH >12) and extracted with EtOAc (3×300 mL). The combined organic layers were washed with saturated aqueous NaCl, dried over MgSO₄, and the filtrate was concentrated to afford the desired product (28.3 g, 70% yield) as a colorless oil. The crude material obtained was used directly without further purification. LC-MS calculated for C₁₂H₁₈NO₂ (M+H)⁺: m/z=208.1. found 208.2.

### Step 2: Methyl (R)-2-((S)—N-benzyl-2-((tert-butoxycarbonyl)amino)propanamido)butanoate

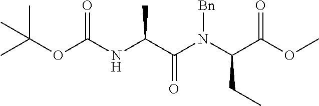 [SMILES: CC[C@H](C(=O)OC)N(Cc1ccccc1)C(=O)[C@H](C)NC(=O)OC(C)(C)C]

To a mixture of methyl (R)-2-(benzylamino)butanoate (18.4 g, 89 mmol) and (tert-butoxycarbonyl)-L-alanine (21.8 g, 115 mmol, Combi-Blocks QA-6543) in N,N-dimethylformamide (100 mL) was added HATU (50.6 g, 133 mmol, Oakwood 023926) followed by N-ethyl-N-isopropylpropan-2-amine (41.9 mL, 240 mmol) and the reaction mixture was stirred at rt overnight. The mixture was diluted with Et₂O (600 mL) and washed with water (200 mL). After phase separation the organic layer was removed and the aqueous layer was extracted with Et₂O (2×200 mL). The combined organic layers were dried over MgSO₄, concentrated, and the crude residue was purified by flash column chromatography (SiO₂, EtOAc/hexanes) to afford the desired product (30 g, 89% yield). LC-MS calculated for C₂₀H₃₁N₂O₅ (M+H)⁺: m/z=379.2. found 379.3.

### Step 3: (3S,6R)-1-Benzyl-6-ethyl-3-methylpiperazine-2,5-dione

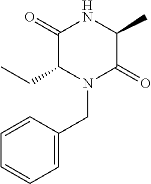 [SMILES: CC[C@@H]1C(=O)N[C@@H](C)C(=O)N1Cc1ccccc1]

To a mixture of methyl (R)-2-((S)—N-benzyl-2-((tert-butoxycarbonyl)amino)propanamido)butanoate (30 g, 79 mmol) in CH₂Cl₂ (200 mL) was added trifluoroacetic acid (50 mL, 649 mmol) and the reaction mixture was stirred at rt overnight. The reaction mixture was concentrated in vacuo. To the crude residue was added MeOH (200 mL) and the reaction mixture was sealed and stirred at 70° C. overnight. After cooling to rt, the reaction mixture was concentrated in vacuo to afford the desired product (27 g). The crude material obtained was used directly without further purification. LC-MS calculated for C₁₄H₁₉N₂O₂ (M+H)⁺: m/z=247.1. found 247.2.

### Step 4: (2R,5S)-1-Benzyl-2-ethyl-5-methylpiperazine

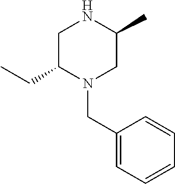 [SMILES: CC[C@@H]1CN[C@@H](C)CN1Cc1ccccc1]

A mixture of (3S,6R)-1-benzyl-6-ethyl-3-methylpiperazine-2,5-dione (Step 3) in THE (200 mL) was cooled to 0° C. in an ice-bath before borane tetrahydrofuran complex (1 M in THF, 375 mL, 375 mmol, Aldrich 176192) was added slowly. The ice-bath was removed and the reaction mixture was stirred at 70° C. for 20 h. After cooling to rt, the reaction mixture was quenched via the slow addition of MeOH (100 mL) followed by 1 M aqueous HCl (112 mL, 112 mmol). The mixture was stirred at 70° C. for an additional 2 h. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄, and concentrated. The crude material obtained was used directly without further purification. LC-MS calculated for C₁₄H₂₃N₂(M+H)⁺: m/z=219.2. found 219.1.

### Step 5: tert-Butyl (2S,5R)-4-benzyl-5-ethyl-2-methylpiperazine-1-carboxylate

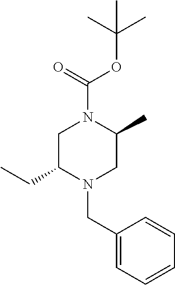 [SMILES: CC[C@@H]1CN(C(=O)OC(C)(C)C)[C@@H](C)CN1Cc1ccccc1]

To a mixture of (2R,5S)-1-benzyl-2-ethyl-5-methylpiperazine (Step 4) in CH₂Cl₂ (150 mL) was added triethylamine (31.3 mL, 225 mmol) and di-tert-butyl dicarbonate (26.1 mL, 112 mmol) and the reaction mixture was stirred at rt overnight. The mixture was diluted with CH₂Cl₂ and washed with water (150 mL) and saturated aqueous NaCl. The organic layer was dried over MgSO₄, concentrated, and the crude residue was purified by flash column chromatography (SiO₂, EtOAc/hexanes) to afford the desired product (22.2 g) as an off-white solid. LC-MS calculated for C₁₉H₃₁N₂O₂ (M+H)⁺: m/z=319.2. found 319.3.

### Step 6: tert-Butyl (2S,5R)-5-ethyl-2-methylpiperazine-1-carboxylate

To a mixture of tert-butyl (2S,5R)-4-benzyl-5-ethyl-2-methylpiperazine-1-carboxylate (22.2 g, 69.7 mmol) in MeOH (170 mL) was added palladium on carbon (10 wt %, 3.2 g, 3 mmol) and the reaction mixture was shaken in a Parr shaker under 50 psi of H₂ (g) for 20 h. The mixture was filtered over a pad of Celite®, and the filter cake was washed with MeOH (170 mL). The filtrate was concentrated and dried under vacuum to afford the desired product (12.5 g, 78% yield). The material obtained was used directly without further purification. LC-MS calculated for C₁₂H₂₅N₂O₂ (M+H)⁺: m/z=229.2. found 229.3.

### Intermediate 42. tert-Butyl (2S,5R)-4-(3,3-difluorocyclobutane-1-carbonyl)-5-ethyl-2-methylpiperazine-1-carboxylate

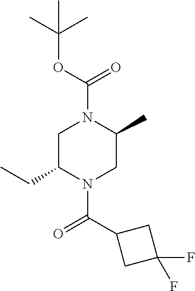 [SMILES: CC[C@@H]1CN(C(=O)OC(C)(C)C)[C@@H](C)CN1C(=O)C1CC(F)(F)C1]

The title compound was prepared according to the procedures described in Intermediate 2, with tert-butyl (2S,5R)-5-ethyl-2-methylpiperazine-1-carboxylate (Intermediate 41) replacing tert-butyl (2S,5R)-2,5-dimethylpiperazine-1-carboxylate. LC-MS calculated for C₁₃H₂₁F₂N₂O₃(M-C₄H₈+H)⁺: m/z=291.2. found 291.1.

### Intermediate 43. (2R,5S)-1-((4-Chlorophenyl)(3,3-difluorocyclobutyl)methyl)-2-ethyl-5-methylpiperazine hydrochloride

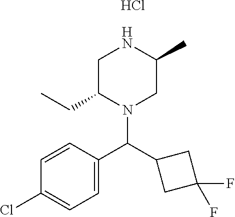 [SMILES: Cl ; CC[C@@H]1CN[C@@H](C)CN1C(c1ccc(Cl)cc1)C1CC(F)(F)C1]

### Step 1: (4-Chlorophenyl)magnesium chloride lithium chloride (1.1 M in THF)

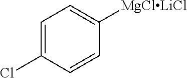

A 1.3 M solution of isopropylmagnesium chloride lithium chloride complex in THE (5.78 mL, 7.52 mmol, Aldrich 656984) was cooled to −78° C. before 1-bromo-4-chlorobenzene (0.96 mL, 8.3 mmol) was added dropwise and the reaction mixture was stirred at −78° C. for 5 min. The reaction mixture was warmed to rt and stirred for an additional 4 h. The mixture obtained was used directly in the next step.

### Step 2: tert-Butyl (2S,5R)-4-((4-chlorophenyl)(3,3-difluorocyclobutyl)methyl)-5-ethyl-2-methylpiperazine-1-carboxylate

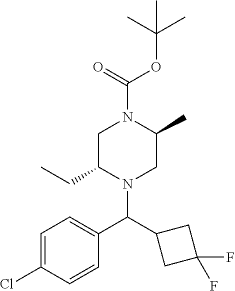 [SMILES: CC[C@@H]1CN(C(=O)OC(C)(C)C)[C@@H](C)CN1C(c1ccc(Cl)cc1)C1CC(F)(F)C1]

A mixture of tert-butyl (2S,5R)-4-(3,3-difluorocyclobutane-1-carbonyl)-5-ethyl-2-methylpiperazine-1-carboxylate (Intermediate 42, 0.800 g, 2.31 mmol) and chlorocarbonylbis(triphenylphosphine)iridium(I) (180 mg, 0.231 mmol, Strem 77-0300) in CH₂Cl₂ (5 mL) was treated with 1,1,3,3-tetramethyldisiloxane (816 μL, 4.62 mmol, Aldrich 235733) and stirred at rt for 25 min. The reaction was cooled to −78° C. and stirred for 5 min before (4-chlorophenyl)magnesium chloride lithium chloride (Step 1, 2.89 mL, 1.1 M in THF, 3.2 mmol) was added dropwise and the reaction mixture was stirred for an additional 5 min. The reaction mixture was warmed to 0° C. and stirred for 3 h. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₃H₃₄ClF₂N₂O₂ (M+H)⁺: m/z=443.2. found 443.3.

### Step 3: (2R,5S)-1-((4-Chlorophenyl)(3,3-difluorocyclobutyl)methyl)-2-ethyl-5-methylpiperazine hydrochloride

A mixture of tert-butyl (2S,5R)-4-((4-chlorophenyl)(3,3-difluorocyclobutyl)methyl)-5-ethyl-2-methylpiperazine-1-carboxylate (Step 2) in THE (15 mL) was treated with HCl (4 M in 1,4-dioxane, 5 mL, 20 mmol, Oakwood 094030) and stirred at 60° C. for 30 min. The mixture was then diluted with diethyl ether and the precipitate was collected by filtration and washed with diethyl ether to afford the desired product as mixture of diastereomers in the form of a white solid. LC-MS calculated for C₁₈H₂₆ClF₂N₂(M+H)⁺: m/z=343.2. found 343.2.

### Intermediate 44. (2R,5S)-1-((3-Chloro-4-fluorophenyl)(3,3-difluorocyclobutyl)methyl)-2,5-dimethylpiperazine hydrochloride

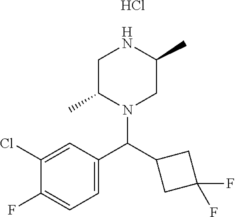 [SMILES: Cl ; C[C@@H]1CN[C@@H](C)CN1C(c1ccc(F)c(Cl)c1)C1CC(F)(F)C1]

### Step 1: tert-Butyl (2S,5R)-4-((3-chloro-4-fluorophenyl)(3,3-difluorocyclobutyl)methyl)-2,5-dimethylpiperazine-1-carboxylate

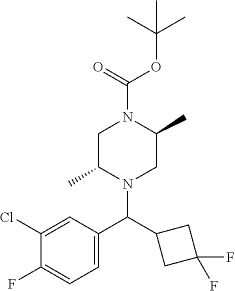 [SMILES: C[C@@H]1CN(C(=O)OC(C)(C)C)[C@@H](C)CN1C(c1ccc(F)c(Cl)c1)C1CC(F)(F)C1]

A mixture of tert-butyl (2S,5R)-4-(3,3-difluorocyclobutane-1-carbonyl)-2,5-dimethylpiperazine-1-carboxylate (Intermediate 2, 1.00 g, 3.01 mmol) and chlorocarbonylbis(triphenylphosphine)iridium(I) (0.235 g, 0.301 mmol, Strem 77-0300) in CH₂Cl₂ (10 mL) was treated with 1,1,3,3-tetramethyldisiloxane (1.06 mL, 6.02 mmol, Aldrich 235733) and stirred at rt for 15 min. Immediate gas evolution was observed, and the yellow color of the catalyst became bleached over the course of 15 min. The reaction was cooled to −78° C. and stirred for 5 min before (3-chloro-4-fluorophenyl)magnesium bromide (7.52 mL, 3.76 mmol, 0.5 M in THF, Aldrich 563676) was added dropwise and the reaction mixture was stirred for an additional 5 min. The reaction mixture was warmed to 0° C. and stirred for 30 min. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₂H₃₁ClF₃N₂O₂ (M+H)⁺: m/z=447.2. found 447.3.

### Step 2: (2R,5S)-1-((3-Chloro-4-fluorophenyl)(3,3-difluorocyclobutyl)methyl)-2,5-dimethylpiperazine hydrochloride

A mixture of tert-butyl (2S,5R)-4-((3-chloro-4-fluorophenyl)(3,3-difluorocyclobutyl)methyl)-2,5-dimethylpiperazine-1-carboxylate (Step 1) in THE (5 mL) was treated with HCl (4 M in 1,4-dioxane, 5 mL, 20 mmol, Oakwood 094030) and stirred at 60° C. for 1 h. The mixture was then diluted with diethyl ether and the precipitate was collected by filtration and washed with diethyl ether to afford the desired product (0.56 g, 54% yield over two steps) as mixture of diastereomers in the form of a white solid. LC-MS calculated for C₁₇H₂₃ClF₃N₂(M+H)⁺: m/z=347.1. found 347.4.

### Intermediate 45. 4-((2S,5R)-2,5-Dimethylpiperazin-1-yl)-2-methyl-1-(((S)-tetrahydrofuran-2-yl)methyl)-1H-[1,2,4]triazolo[3,4-b]purine hydrochloride

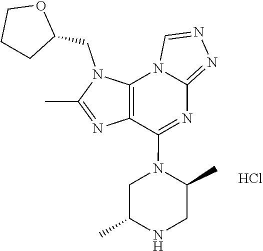 [SMILES: Cc1nc2c(N3C[C@@H](C)NC[C@@H]3C)nc3nncn3c2n1C[C@@H]1CCCO1 ; Cl]

### Step 1. tert-Butyl (2R,5S)-4-(2-chloro-8-methyl-9-(((S)-tetrahydrofuran-2-yl)methyl)-9H-purin-6-yl)-2,5-dimethylpiperazine-1-carboxylate

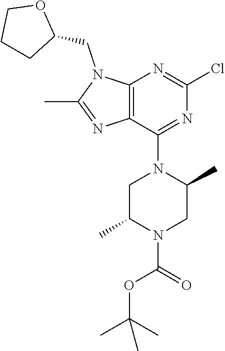 [SMILES: Cc1nc2c(N3C[C@@H](C)N(C(=O)OC(C)(C)C)C[C@@H]3C)nc(Cl)nc2n1C[C@@H]1CCCO1]

To a mixture of (S)-2,6-dichloro-8-methyl-9-((tetrahydrofuran-2-yl)methyl)-9H-purine (Intermediate 5, 1.02 g, 5.0 mmol) and tert-butyl (2R,5S)-2,5-dimethylpiperazine-1-carboxylate (1.07 g, 5.0 mmol) in MeCN (10.0 mL) was added potassium carbonate (1.38 g, 10.0 mmol) and the reaction mixture was stirred at 90° C. overnight. After cooling to rt, the reaction mixture was filtered over Celite and the filtrate was concentrated in vacuo. The crude residue was purified directly by flash column chromatography (24 g SiO₂, EtOAc/hexanes) to afford the desired product as a light yellow solid. LC-MS calculated for C₂₂H₃₄ClN₆O₃(M+H)⁺: m/z=465.2. found 465.3.

### Step 2. tert-Butyl (2R,5S)-4-(2-hydrazineyl-8-methyl-9-(((S)-tetrahydrofuran-2-yl)methyl)-9H-purin-6-yl)-2,5-dimethylpiperazine-1-carboxylate

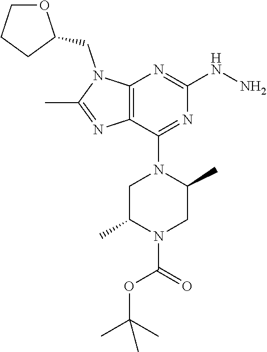 [SMILES: Cc1nc2c(N3C[C@@H](C)N(C(=O)OC(C)(C)C)C[C@@H]3C)nc(NN)nc2n1C[C@@H]1CCCO1]

To a mixture of tert-butyl (2R,5S)-4-(2-chloro-8-methyl-9-(((S)-tetrahydrofuran-2-yl)methyl)-9H-purin-6-yl)-2,5-dimethylpiperazine-1-carboxylate (465 mg, 1.0 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (85 mg, 0.10 mmol), and cesium carbonate (1.63 g, 5.0 mmol) was added a 1 molar solution of hydrazine in THE (5.0 mL, 5.0 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₂H₃₇N₈O₃ (M+H)⁺: m/z=461.3. found 461.3.

### Step 3. tert-Butyl (2R,5S)-2,5-dimethyl-4-(2-methyl-1-(((S)-tetrahydrofuran-2-yl)methyl)-1H-[1,2,4]triazolo[3,4-b]purin-4-yl)piperazine-1-carboxylate

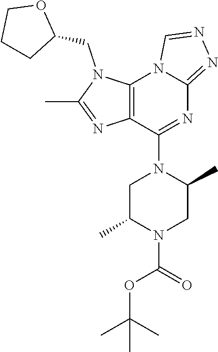 [SMILES: Cc1nc2c(N3C[C@@H](C)N(C(=O)OC(C)(C)C)C[C@@H]3C)nc3nncn3c2n1C[C@@H]1CCCO1]

A mixture of tert-butyl (2R,5S)-4-(2-hydrazineyl-8-methyl-9-(((S)-tetrahydrofuran-2-yl)methyl)-9H-purin-6-yl)-2,5-dimethylpiperazine-1-carboxylate (Step 2), triethyl orthoformate (2.0 mL, 12.0 mmol), and AcOH (0.4 mL, 7.0 mmol) was stirred at 90° C. overnight. After cooling to rt, the reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (12 g SiO₂, MeOH/CH₂Cl₂) to give the desired product as a yellow solid. LC-MS calculated for C₂₃H₃₅N₈O₃ (M+H)⁺: m/z=471.3. found 471.3.

### Step 4. 4-((2S,5R)-2,5-Dimethylpiperazin-1-yl)-2-methyl-1-(((S)-tetrahydrofuran-2-yl)methyl)-1H-[1,2,4]triazolo[3,4-b]purine hydrochloride

To a mixture of tert-butyl (2R,5S)-2,5-dimethyl-4-(2-methyl-1-(((S)-tetrahydrofuran-2-yl)methyl)-1H-[1,2,4]triazolo[3,4-b]purin-4-yl)piperazine-1-carboxylate (Step 3) in CH₂Cl₂ (1.0 mL) was added a 4 molar solution of HCl in 1,4-dioxane (0.50 mL, 2.0 mmol), and the reaction mixture was allowed to stir at rt for 4 h. The reaction mixture was concentrated in vacuo to give desired product as a light yellow solid. LC-MS calculated for C₁₈H₂₇N₈O (M+H)⁺: m/z=371.2. found 371.3.

### Intermediate 48. (2R,5S)-1-((4-Chloro-3-fluorophenyl)(3,3-difluorocyclobutyl)methyl)-2,5-dimethylpiperazine hydrochloride

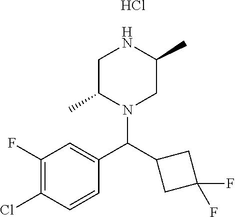 [SMILES: Cl ; C[C@@H]1CN[C@@H](C)CN1C(c1ccc(Cl)c(F)c1)C1CC(F)(F)C1]

### Step 1: (4-chloro-3-fluorophenyl)magnesium chloride lithium chloride (1.1 M in THF)

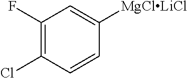

A 1.3 M solution of isopropylmagnesium chloride lithium chloride complex in THE (800 μL, 1.04 mmol, Aldrich 656984) was cooled to −78° C. before 1-chloro-2-fluoro-4-iodobenzene (146 μL, 1.14 mmol, Apollo Scientific PC9033) was added dropwise and the reaction mixture was stirred at −78° C. for 1 h. The reaction mixture was warmed to rt and stirred for 1 h. The mixture obtained was used directly in the next step.

### Step 2: tert-Butyl (2S,5R)-4-((4-chloro-3-fluorophenyl)(3,3-difluorocyclobutyl)methyl)-2,5-dimethylpiperazine-1-carboxylate

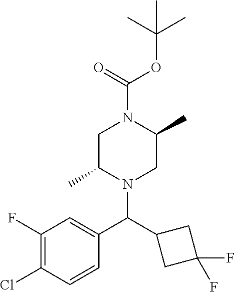 [SMILES: C[C@@H]1CN(C(=O)OC(C)(C)C)[C@@H](C)CN1C(c1ccc(Cl)c(F)c1)C1CC(F)(F)C1]

A mixture of tert-butyl (2S,5R)-4-(3,3-difluorocyclobutane-1-carbonyl)-2,5-dimethylpiperazine-1-carboxylate (Intermediate 2, 250 mg, 0.752 mmol) and chlorocarbonylbis(triphenylphosphine)iridium(I) (59 mg, 0.075 mmol, Strem 77-0300) in CH₂Cl₂ (5 mL) was treated with 1,1,3,3-tetramethyldisiloxane (226 μL, 1.50 mmol, Aldrich 235733) and stirred at rt for 15 min. Immediate gas evolution was observed, and the yellow color of the catalyst became bleached over the course of 15 min. Additional chlorocarbonylbis(triphenylphosphine)iridium(I) (59 mg, 0.075 mmol) and 1,1,3,3-tetramethyldisiloxane (226 μL, 1.50 mmol) was added and the reaction mixture was stirred at rt for an additional 15 min. The reaction was cooled to −78° C. and stirred for 5 min before (4-chloro-3-fluorophenyl)magnesium chloride lithium chloride (Step 1, 855 μL, 1.1 M in THF, 0.94 mmol) was added dropwise and the reaction mixture was stirred for an additional 5 min. The reaction mixture was warmed to 0° C. and stirred for 30 min. The mixture was quenched with saturated aqueous NH₄Cl. After warming to rt, the organic layer was removed and the aqueous layer was extracted with CH₂Cl₂. The combined organic layers were dried over MgSO₄ and the filtrate was concentrated in vacuo. The crude residue was purified by flash column chromatography (SiO₂, EtOAc/hexanes) to afford the desired product as a mixture of diastereomers. LC-MS calculated for C₂₂H₃₁ClF₃N₂O₂ (M+H)⁺: m/z=447.2. found 447.2.

### Step 3: (2R,5S)-1-((4-chloro-3-fluorophenyl)(3,3-difluorocyclobutyl)methyl)-2,5-dimethylpiperazine hydrochloride

A mixture of tert-butyl (2S,5R)-4-((4-chloro-3-fluorophenyl)(3,3-difluorocyclobutyl)methyl)-2,5-dimethylpiperazine-1-carboxylate (Step 2) in THE (15 mL) was treated with HCl (4 M in 1,4-dioxane, 5 mL, 20 mmol, Oakwood 094030) and stirred at 60° C. for 1 h. After cooling to rt, the mixture was diluted with diethyl ether (5 mL) and slurried for 30 min. The resulting precipitate was collected by filtration, washed with diethyl ether, and dried under vacuum to afford the desired product as a mixture of diastereomers in the form of a white solid. LC-MS calculated for C₁₇H₂₃ClF₃N₂(M+H)⁺: m/z=347.2. found 347.1.

### Intermediate 50. (S)-(Tetrahydrofuran-2-yl)methyl methanesulfonate

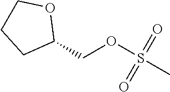

A mixture of (S)-(tetrahydrofuran-2-yl)methanol (2.00 g, 19.6 mmol, BLD Pharmatech BD48351) and N-ethyl-N-isopropylpropan-2-amine (5.12 mL, 29.4 mmol) in CH₂Cl₂ (15 mL) was purged with N₂ and cooled to 0° C. before methanesulfonyl chloride (1.97 mL, 25.5 mmol) was added dropwise. The reaction mixture was allowed to warm to rt and stirred for 30 mins. The mixture was quenched with saturated aqueous NaHCO₃, the organic layer was removed, and the aqueous layer was extracted with CH₂Cl₂. The combined organic layers were dried over MgSO₄, filtered, and concentrated under reduced pressure to afford the desired product (3.39 g, 96% yield) as a light orange oil that was used directly without further purification. ¹H NMR (400 MHz, CDCl₃) δ 4.28-4.20 (m, 1H), 4.20-4.13 (m, 2H), 3.88 (dt, J=8.4, 6.6 Hz, 1H), 3.80 (dt, J=8.2, 6.6 Hz, 1H), 3.05 (s, 3H), 2.08-1.97 (m, 1H), 1.97-1.87 (m, 2H), 1.73-1.63 (m, 1H).

### Intermediate 52. 5-Chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine

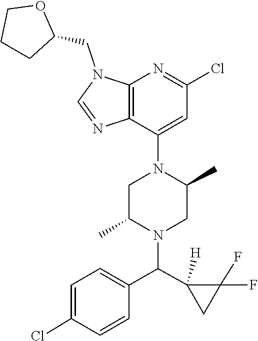 [SMILES: C[C@@H]1CN(c2cc(Cl)nc3c2ncn3C[C@@H]2CCCO2)[C@@H](C)CN1C(c1ccc(Cl)cc1)[C@@H]1CC1(F)F]

### Step 1. 6-Chloro-4-((2S,5R)-4-((4-chlorophenyl)((S)-2,2-difluorocyclopropyl)methyl)-2,5-dimethylpiperazin-1-yl)-3-nitropyridin-2-amine

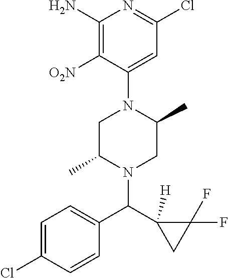 [SMILES: C[C@@H]1CN(c2cc(Cl)nc(N)c2[N+](=O)[O-])[C@@H](C)CN1C(c1ccc(Cl)cc1)[C@@H]1CC1(F)F]

A mixture of (2R,5S)-1-((4-chlorophenyl)((S)-2,2-difluorocyclopropyl)methyl)-2,5-dimethylpiperazine hydrochloride (Intermediate 16, 0.105 g, 0.300 mmol), 4,6-dichloro-3-nitropyridin-2-amine (62.0 mg, 0.300 mmol PharmaBlock PBT0266), and N,N-diisopropylethylamine (0.157 mL, 0.900 mmol) in MeCN (1 mL) was stirred at 90° C. overnight. After cooling to room temperature, the mixture was concentrated in vacuo and used without further purification. LC-MS calculated for C₂₁H₂₄Cl₂F₂N₅O₂(M+H)⁺: 15 m/z=486.1. found 486.1.

### Step 2. 6-Chloro-4-((2S,5R)-4-((4-chlorophenyl)((S)-2,2-difluorocyclopropyl)methyl)-2,5-dimethylpiperazin-1-yl)pyridine-2,3-diamine

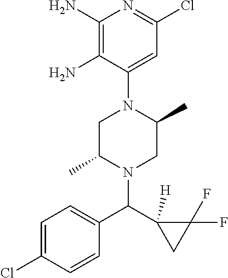 [SMILES: C[C@@H]1CN(c2cc(Cl)nc(N)c2N)[C@@H](C)CN1C(c1ccc(Cl)cc1)[C@@H]1CC1(F)F]

A mixture of 6-chloro-4-((2S,5R)-4-((4-chlorophenyl)((S)-2,2-difluorocyclopropyl)methyl)-2,5-dimethylpiperazin-1-yl)-3-nitropyridin-2-amine (Step 1), 4,4′-dipyridyl (4.7 mg, 0.030 mmol, Aldrich 289426), and tetrahydroxydiboron (81 mg, 0.90 mmol, BLD Pharmatech BD288251) in DMF (0.6 mL) was stirred at room temperature for 10 minutes. The mixture was diluted with CH₂Cl₂ and water and filtered through a pad of Celite®. The layers were separated, and the aqueous layer was extracted with CH₂Cl₂. The combined organic layers were dried over MgSO₄ and concentrated in vacuo. The crude product was used without further purification. LC-MS calculated for C₂₁H₂₆Cl₂F₂N₅ (M+H)⁺: m/z=456.2. found 456.1.

### Step 3. 5-Chloro-7-((2S,5R)-4-((4-chlorophenyl)((S)-2,2-difluorocyclopropyl)methyl)-2,5-dimethylpiperazin-1-yl)-3H-imidazo[4,5-b]pyridine

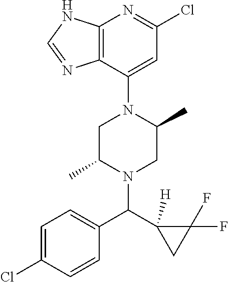 [SMILES: C[C@@H]1CN(c2cc(Cl)nc3[nH]cnc23)[C@@H](C)CN1C(c1ccc(Cl)cc1)[C@@H]1CC1(F)F]

A mixture of 6-chloro-4-((2S,5R)-4-((4-chlorophenyl)((S)-2,2-difluorocyclopropyl)methyl)-2,5-dimethylpiperazin-1-yl)pyridine-2,3-diamine (Step 2), triethyl orthoformate (0.125 mL, 0.750 mmol), and acetic acid (0.429 mL, 7.50 mmol) was stirred at 95° C. for 30 minutes. The mixture was cooled to room temperature, diluted with CH₂Cl₂, and quenched with saturated aq. NaHCO₃. The layers were separated and the aqueous layer was extracted with CH₂Cl₂. The combined organic layers were dried over MgSO₄ and concentrated in vacuo. The crude product was used without further purification. LC-MS calculated for C₂₂H₂₄Cl₂F₂N₅ (M+H)⁺: m/z=466.1. found 466.2.

### Step 4: 5-Chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine

A mixture of 5-chloro-7-((2S,5R)-4-((4-chlorophenyl)((S)-2,2-difluorocyclopropyl)methyl)-2,5-dimethylpiperazin-1-yl)-3H-imidazo[4,5-b]pyridine (Step 3), (S)-(tetrahydrofuran-2-yl)methyl methanesulfonate (Intermediate 50, 0.135 g, 0.750 mmol), and cesium carbonate (0.489, 1.50 mmol) in MeCN (1.0 mL) was stirred at 95° C. for 4 h. The mixture was cooled to room temperature, filtered, and concentrated in vacuo. The residue was purified by flash column chromatography (24 g SiO₂, EtOAc/hexanes) to give the title compound (44.9 mg, 27% yield) as a white solid. LC-MS calculated for C₂₇H₃₂Cl₂F₂N₅O (M+H)⁺: m/z=550.2. found 550.2.

### Intermediate 54. tert-Butyl (2S,5R)-4-(4-chlorobenzoyl)-2,5-dimethylpiperazine-1-carboxylate

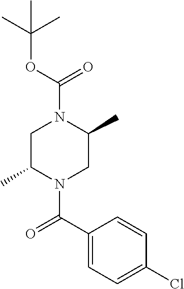 [SMILES: C[C@@H]1CN(C(=O)OC(C)(C)C)[C@@H](C)CN1C(=O)c1ccc(Cl)cc1]

The title compound was prepared according to the procedure outlined for Intermediate 9, with 4-chlorobenzoyl chloride (Aldrich 111902) replacing isovaleryl chloride. LC-MS calculated for C₁₄H₁₈ClN₂O₃(M-C₄H₈+H)⁺: m/z=297.1. found 297.1.

### Intermediate 55. (2R,5S)-1-(1-(4-Chlorophenyl)propyl)-2,5-dimethylpiperazine hydrochloride

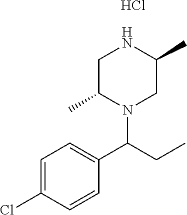 [SMILES: Cl ; CCC(c1ccc(Cl)cc1)N1C[C@H](C)NC[C@H]1C]

The title compound was prepared according to the procedures outlined for Intermediate 10, with tert-butyl (2S,5R)-4-(4-chlorobenzoyl)-2,5-dimethylpiperazine-1-carboxylate (Intermediate 54) replacing tert-butyl (2S,5R)-2,5-dimethyl-4-(3-methylbutanoyl)piperazine-1-carboxylate and ethylmagnesium bromide (Aldrich 752126) replacing (4-chlorophenyl)magnesium bromide. The title compound was isolated as a mixture of diastereomers. LC-MS calculated for C₁₅H₂₄ClN₂ (M+H)⁺: m/z=267.2. found 267.2.

### Intermediate 58. 5-Chloro-7-((2S,5R)-4-((4-chlorophenyl)(3,3-difluorocyclobutyl)methyl)-2,5-dimethylpiperazin-1-yl)-3-(((S)-tetrahydrofuran-2-yl)methyl)-3H-imidazo[4,5-b]pyridine

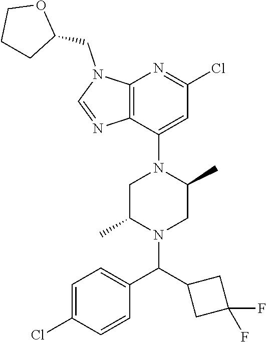 [SMILES: C[C@@H]1CN(c2cc(Cl)nc3c2ncn3C[C@@H]2CCCO2)[C@@H](C)CN1C(c1ccc(Cl)cc1)C1CC(F)(F)C1]

The title compound was prepared according to the procedures outlined in Intermediate 52, with (2R,5S)-1-((4-chlorophenyl)(3,3-difluorocyclobutyl)methyl)-2,5-dimethylpiperazine hydrochloride (Intermediate 39) replacing (2R,5S)-1-((4-chlorophenyl)((S)-2,2-difluorocyclopropyl)methyl)-2,5-dimethylpiperazine hydrochloride in Step 1. The title compound was isolated as a mixture of diastereomers. LC-MS calculated for C₂₈H₃₄Cl₂F₂N₅O (M+H)⁺: m/z=564.2. found 564.1.

### Intermediate 59. 5-Chloro-7-((2S,5R)-4-((4-chlorophenyl)(3,3-difluorocyclobutyl)methyl)-2,5-dimethylpiperazin-1-yl)-2-methyl-3-(((S)-tetrahydrofuran-2-yl)methyl)-3H-imidazo[4,5-b]pyridine

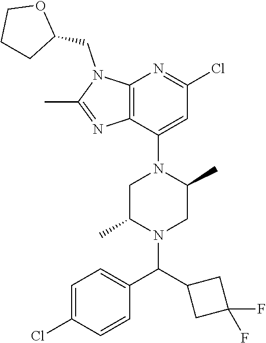 [SMILES: Cc1nc2c(N3C[C@@H](C)N(C(c4ccc(Cl)cc4)C4CC(F)(F)C4)C[C@@H]3C)cc(Cl)nc2n1C[C@@H]1CCCO1]

The title compound was prepared according to the procedures outlined in Intermediate 52, with (2R,5S)-1-((4-chlorophenyl)(3,3-difluorocyclobutyl)methyl)-2,5-dimethylpiperazine hydrochloride (Intermediate 39) replacing (2R,5S)-1-((4-chlorophenyl)((S)-2,2-difluorocyclopropyl)methyl)-2,5-dimethylpiperazine hydrochloride in Step 1 and triethyl orthoacetate replacing triethyl orthoformate in Step 3. The title compound was isolated as a mixture of diastereomers. LC-MS calculated for C₂₉H₃₆Cl₂F₂N₅O (M+H)⁺: m/z=578.2. found 578.3.

### Intermediate 83. (2R,5S)-1-(Bis(4-chlorophenyl)methyl)-2,5-dimethylpiperazine hydrochloride

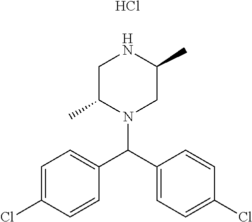 [SMILES: Cl ; C[C@@H]1CN[C@@H](C)CN1C(c1ccc(Cl)cc1)c1ccc(Cl)cc1]

### Step 1: tert-Butyl (2S,5R)-4-(bis(4-chlorophenyl)methyl)-2,5-dimethylpiperazine-1-carboxylate

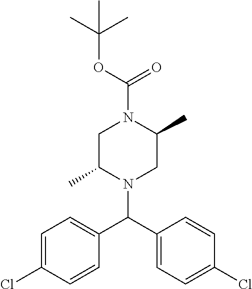 [SMILES: C[C@@H]1CN(C(=O)OC(C)(C)C)[C@@H](C)CN1C(c1ccc(Cl)cc1)c1ccc(Cl)cc1]

In a 20 mL microwave vial with a stir bar, a mixture of tert-butyl (2S,5R)-2,5-dimethylpiperazine-1-carboxylate (1.11 g, 5.19 mmol, Combi-Blocks OR-8588), 4,4′-(chloromethylene)bis(chlorobenzene) (1.41 g, 5.19 mmol, A2B Chem AC49945), and N,N-diisopropylethylamine (1.81 mL, 10.4 mmol) in MeCN (13 mL) was irradiated at 115° C. in a microwave reactor for 4 h. A second reaction was set up in parallel in a separate vessel, and after cooling to rt the two reaction mixtures were combined and concentrated in vacuo. The crude residue was diluted with EtOAc and water and the layers were separated. The organic layer was removed, and the aqueous layer was extracted with EtOAc. The combined organic layers were washed with brine, dried over MgSO₄, and concentrated in vacuo. The crude residue was purified by flash column chromatography (40 g SiO₂, EtOAc/hexanes) to give the title compound as a white solid. LC-MS calculated for C₂₄H₃₁Cl₂N₂O₂ (M+H)⁺: m/z=449.2. found 449.2.

### Step 2: (2R,5S)-1-(Bis(4-chlorophenyl)methyl)-2,5-dimethylpiperazine hydrochloride

A mixture of tert-butyl (2S,5R)-4-(bis(4-chlorophenyl)methyl)-2,5-dimethylpiperazine-1-carboxylate (Step 1) in THE (26 mL) was treated with HCl (4 M in 1,4-dioxane, 26 mL, 104 mmol) and the reaction mixture was stirred at 60° C. for 1 h. After cooling to rt, the mixture was diluted with diethyl ether (100 mL). The solid precipitate that formed was collected by filtration, washed with diethyl ether, and dried under vacuum to give the title compound (2.44 g, 61% yield over two steps) as a white solid. LC-MS calculated for C₁₉H₂₃Cl₂N₂(M+H)⁺: m/z=349.1. found 349.2.

### Intermediate 84. (2R,5S)-1-(Bis(4-fluorophenyl)methyl)-2,5-dimethylpiperazine hydrochloride

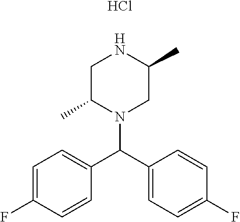 [SMILES: Cl ; C[C@@H]1CN[C@@H](C)CN1C(c1ccc(F)cc1)c1ccc(F)cc1]

### Step 1. tert-Butyl (2S,5R)-4-(bis(4-fluorophenyl)methyl)-2,5-dimethylpiperazine-1-carboxylate

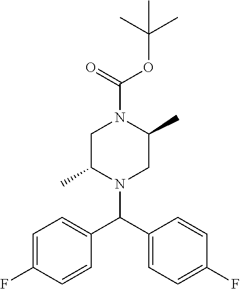 [SMILES: C[C@@H]1CN(C(=O)OC(C)(C)C)[C@@H](C)CN1C(c1ccc(F)cc1)c1ccc(F)cc1]

A mixture of tert-butyl (2S,5R)-2,5-dimethylpiperazine-1-carboxylate (15.0 g, 70 mmol, Combi-Blocks OR-8588), 4,4′-(chloromethylene)bis(fluorobenzene) (19.2 g, 80 mmol, Combi-Blocks QA-4728) and N-ethyl-N-isopropylpropan-2-amine (37 mL, 210 mmol) in CH₃CN (175 mL) was stirred at 85° C. overnight. After cooling to rt, the reaction mixture was concentrated in vacuo and the residue was dissolved in EtOAc and washed with water and brine. The organic phase was dried over MgSO₄, filtered, and concentrated and the crude residue was purified using flash column chromatography (330 g SiO₂, EtOAc/hexanes) to afford tert-butyl (2S,5R)-4-(bis(4-fluorophenyl)methyl)-2,5-dimethylpiperazine-1-carboxylate (26.0 g, 89% yield) as a light yellow waxy solid. LC-MS calculated for C₂₄H₃₁F₂N₂O₂(M+H)⁺: m/z=417.2. found 417.1.

### Step 2. (2R,5S)-1-(Bis(4-fluorophenyl)methyl)-2,5-dimethylpiperazine hydrochloride

To a mixture of tert-butyl (2S,5R)-4-(bis(4-fluorophenyl)methyl)-2,5-dimethylpiperazine-1-carboxylate (1.86 g, 4.5 mmol) in THE (25 mL) was added a 4 molar solution of HCl in 1,4-dioxane (6.25 mL, 25.0 mmol) and the reaction mixture was purged with N₂ and stirred at 80° C. for 4 h. After cooling to rt, the reaction mixture was diluted with Et₂O (25 mL) and hexanes (50 mL) and slurried for 30 mins. The solid precipitate was collected via filtration, washed with Et₂O and hexanes, and dried under vacuum to afford the desired product (1.34 g, 85% yield) as a white solid. LC-MS calculated for C₁₉H₂₃F₂N₂ (M+H)⁺: m/z=317.2. found 317.2.

### Example 1. 4-((2S,5R)-4-(Bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine (i.e., “Compound 1”)

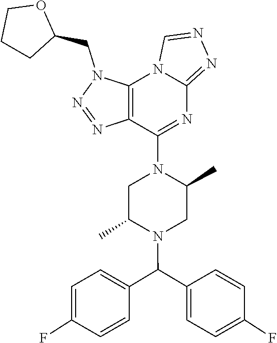 [SMILES: C[C@@H]1CN(c2nc3nncn3c3c2nnn3C[C@H]2CCCO2)[C@@H](C)CN1C(c1ccc(F)cc1)c1ccc(F)cc1]

### Step 1: 6-((2S,5R)-4-(Bis(4-fluorophenyl)methyl)-2,5-dimethylpiperazin-1-yl)-2-chloro-5-nitro-N—(((R)-tetrahydrofuran-2-yl)methyl)pyrimidin-4-amine

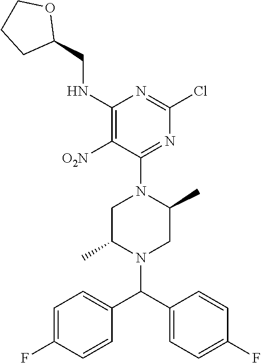 [SMILES: C[C@@H]1CN(c2nc(Cl)nc(NC[C@H]3CCCO3)c2[N+](=O)[O-])[C@@H](C)CN1C(c1ccc(F)cc1)c1ccc(F)cc1]

A mixture of 2,4,6-trichloro-5-nitropyrimidine (2.00 g, 8.76 mmol, Combi-Blocks, ST-3909) and (2R,5S)-1-(bis(4-fluorophenyl)methyl)-2,5-dimethylpiperazine hydrochloride (Intermediate 84, 3.41 g, 9.66 mmol) in CH₃CN (100 mL) was cooled to 0° C. in an ice-bath before N-ethyl-N-isopropylpropan-2-amine (6.12 mL, 35.0 mmol) was added and the reaction mixture was stirred at 0° C. for 30 min. To the mixture was added (R)-(tetrahydrofuran-2-yl)methanamine (0.886 g, 8.76 mmol, BLD Pharmatech, BD46980) and the reaction mixture was warmed to rt and stirred for 30 min. The mixture was diluted with saturated aqueous NaHCO₃ and extracted with EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₈H₃₂ClF₂N₆O₃ (M+H)⁺: m/z=573.2. found 573.2.

### Step 2. 6-((2S,5R)-4-(Bis(4-fluorophenyl)methyl)-2,5-dimethylpiperazin-1-yl)-2-chloro-N⁴—(((R)-tetrahydrofuran-2-yl)methyl)pyrimidine-4,5-diamine

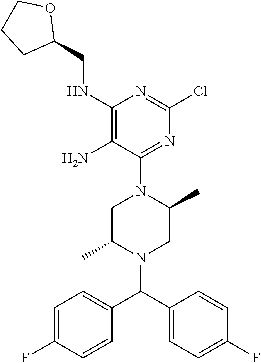 [SMILES: C[C@@H]1CN(c2nc(Cl)nc(NC[C@H]3CCCO3)c2N)[C@@H](C)CN1C(c1ccc(F)cc1)c1ccc(F)cc1]

To a mixture of 6-((2S,5R)-4-(bis(4-fluorophenyl)methyl)-2,5-dimethylpiperazin-1-yl)-2-chloro-5-nitro-N—(((R)-tetrahydrofuran-2-yl)methyl)pyrimidin-4-amine (Step 1) in saturated aqueous NH₄Cl (20 mL)/MeOH(20 mL)/THF(20 mL) was added iron (1.95 g, 35.0 mmol) and the reaction mixture was stirred at 65° C. overnight. After cooling to rt, the reaction mixture was diluted with EtOAc (100 mL) and saturated aqueous NaHCO₃ and the resulting mixture was filtered over a pad of Celite. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₈H₃₄ClF₂N₆O (M+H)⁺: m/z=543.2. found 543.2.

### Step 3. 7-((2S,5R)-4-(Bis(4-fluorophenyl)methyl)-2,5-dimethylpiperazin-1-yl)-5-chloro-3-(((R)-tetrahydrofuran-2-yl)methyl)-3H-[1,2,3]triazolo[4,5-d]pyrimidine

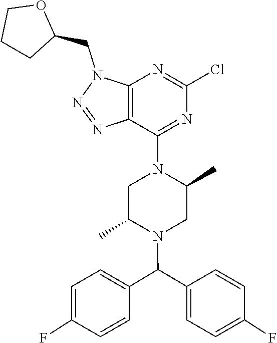 [SMILES: C[C@@H]1CN(c2nc(Cl)nc3c2nnn3C[C@H]2CCCO2)[C@@H](C)CN1C(c1ccc(F)cc1)c1ccc(F)cc1]

To a mixture of 6-((2S,5R)-4-(bis(4-fluorophenyl)methyl)-2,5-dimethylpiperazin-1-yl)-2-chloro-N⁴—(((R)-tetrahydrofuran-2-yl)methyl)pyrimidine-4,5-diamine (Step 2) and AcOH (2.0 mL, 35 mmol) in water (20 mL) and THE (20 mL) was added sodium nitrite (2.42 g, 35.0 mmol) and the reaction mixture was stirred at rt for 30 min. The mixture was diluted with EtOAc (100 mL) and the aqueous layer was adjusted to pH=8 with saturated aqueous NaHCO₃. The organic layer was removed, and the aqueous layer was extracted with EtOAc. The organic phases were combined, dried over MgSO₄, filtered, and concentrated under reduced pressure. The crude residue was purified by flash column chromatography (SiO₂, 0-10% EtOAc/CH₂Cl₂) to afford the desired product (2.8 g, 58% yield over 3 steps) as a yellow solid. LC-MS calculated for C₂₈H₃₁ClF₂N₇O (M+H)⁺: m/z=554.2. found 554.2.

### Step 4. 7-((2S,5R)-4-(Bis(4-fluorophenyl)methyl)-2,5-dimethylpiperazin-1-yl)-5-hydrazineyl-3-(((R)-tetrahydrofuran-2-yl)methyl)-3H-[1,2,3]triazolo[4,5-d]pyrimidine

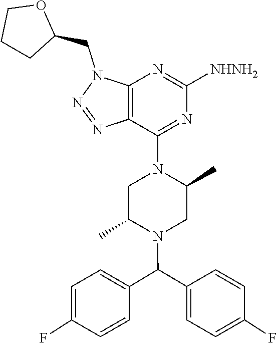 [SMILES: C[C@@H]1CN(c2nc(NN)nc3c2nnn3C[C@H]2CCCO2)[C@@H](C)CN1C(c1ccc(F)cc1)c1ccc(F)cc1]

To a mixture of 7-((2S,5R)-4-(bis(4-fluorophenyl)methyl)-2,5-dimethylpiperazin-1-yl)-5-chloro-3-(((R)-tetrahydrofuran-2-yl)methyl)-3H-[1,2,3]triazolo[4,5-d]pyrimidine (2.8 g, 5.1 mmol), methanesulfonato(2-(di-t-butylphosphino)-3,6-dimethoxy-2′,4′,6′-tri-1-propyl-1,1′-biphenyl)(2′-amino-1,1′-biphenyl-2-yl)palladium(II) (0.374 g, 0.438 mmol, Aldrich 745979) and cesium carbonate (2.85 g, 8.76 mmol) in 1,4-dioxane (100 mL) was added hydrazine (0.561 g, 17.5 mmol) and the mixture was purged with nitrogen and stirred at 90° C. for 1 h. After cooling to rt, the reaction mixture was diluted with CH₂Cl₂ and filtered over a pad of Celite. The filtrate was concentrated under reduced pressure, and the crude residue was purified by flash column chromatography (SiO₂, 0-5% MeOH/CH₂Cl₂) to afford the desired product (2.5 g, 90% yield) as a yellow solid. LC-MS calculated for C₂₈H₃₄F₂N₉O (M+H)⁺: m/z=550.3. found 550.7.

### Step 5. 4-((2S,5R)-4-(Bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine

To a mixture of 7-((2S,5R)-4-(bis(4-fluorophenyl)methyl)-2,5-dimethylpiperazin-1-yl)-5-hydrazineyl-3-(((R)-tetrahydrofuran-2-yl)methyl)-3H-[1,2,3]triazolo[4,5-d]pyrimidine (2.5 g, 4.5 mmol) and AcOH (2.0 mL, 35 mmol) was added triethyl orthoformate (6.49 g, 43.8 mmol) and the reaction mixture was stirred at 95° C. for 1 h. After cooling to rt, the reaction mixture was concentrated under reduced pressure, and to the crude residue was added CH₃CN (10 mL) and saturated aqueous NaHCO₃. The mixture was extracted with EtOAc (3×100 mL), and the combined organic phases were dried over MgSO₄, filtered, and concentrated under reduced pressure. The crude residue was purified by flash column chromatography (SiO₂, 0-70% EtOAc/CH₂Cl₂ followed by 5% MeOH/CH₂Cl₂) to afford the desired product (1.8 g, 71% yield) as a white solid. The product was recrystallized from CH₂Cl₂/MTBE/hexanes (20 mL/20 mL/20 mL) and the solid precipitate was filtered, washed with MTBE/hexanes (1:3), and dried under vacuum to afford the desired product (1.1 g). LC-MS calculated for C₂₉H₃₂F₂N₉O (M+H)⁺: m/z=560.3. found 560.3. ¹H NMR (600 MHz, DMSO-d₆) (mixture of rotamers) δ 9.32 (s, 1H), 7.64-7.57 (m, 4H), 7.19-7.13 (m, 4H), 5.86 (m, 0.5H), 5.59-5.48 (m, 0.5H), 5.13-5.06 (m, 1.5H), 4.93-4.86 (m, 1H), 4.68 (s, 1H), 4.66-4.61 (m, 0.5H), 4.29-4.22 (m, 1H), 3.97-3.84 (m, 0.5H), 3.59-3.46 (m, 2.5H), 3.23-3.11 (m, 1H), 2.88-2.78 (m, 0.5H), 2.77-2.68 (m, 0.5H), 2.48-2.43 (m, 1H), 2.14-2.06 (m, 1H), 1.82-1.70 (m, 3H), 1.52-1.44 (m, 3H), 0.93 (d, J=6.5 Hz, 3H).

### Example 2. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine (i.e. “Compound 2”)

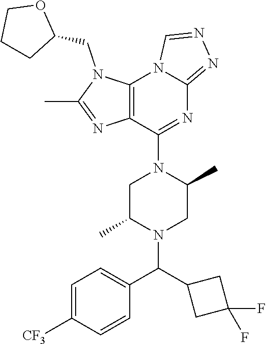

### Step 1. 2-Chloro-6-((2S,5R)-4-((3,3-difluorocyclobutyl)(4-(trifluoromethyl)phenyl)methyl)-2,5-dimethylpiperazin-1-yl)-8-methyl-9-(((S)-tetrahydrofuran-2-yl)methyl)-9H-purine

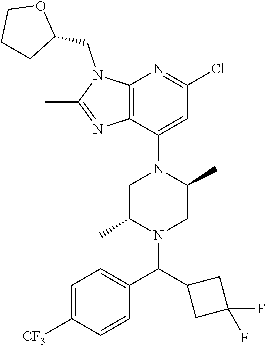 [SMILES: Cc1nc2c(N3C[C@@H](C)N(C(c4ccc(C(F)(F)F)cc4)C4CC(F)(F)C4)C[C@@H]3C)cc(Cl)nc2n1C[C@@H]1CCCO1]

To a mixture of (S)-2,6-dichloro-8-methyl-9-((tetrahydrofuran-2-yl)methyl)-9H-purine (Intermediate 5, 1.44 g, 5.01 mmol) and (2R,5S)-1-((3,3-difluorocyclobutyl)(4-(trifluoromethyl)phenyl)methyl)-2,5-dimethylpiperazine hydrochloride (Intermediate 3, 2.00 g, 5.01 mmol) in 1-butanol (8 mL) was added N,N-diisopropylethylamine (2.63 mL, 15.0 mmol) and the mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₉H₃₅ClF₅N₆O (M+H)⁺: m/z=613.3. found 613.3.

### Step 2. 6-((2S,5R)-4-((3,3-Difluorocyclobutyl)(4-(trifluoromethyl)phenyl)methyl)-2,5-dimethylpiperazin-1-yl)-2-hydrazineyl-8-methyl-9-(((S)-tetrahydrofuran-2-yl)methyl)-9H-purine

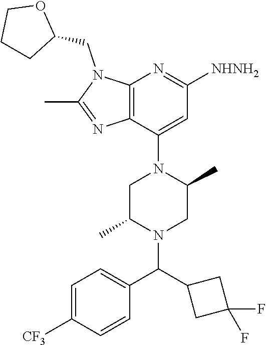 [SMILES: Cc1nc2c(N3C[C@@H](C)N(C(c4ccc(C(F)(F)F)cc4)C4CC(F)(F)C4)C[C@@H]3C)cc(NN)nc2n1C[C@@H]1CCCO1]

To a mixture of 2-chloro-6-((2S,5R)-4-((3,3-difluorocyclobutyl)(4-(trifluoromethyl)phenyl)methyl)-2,5-dimethylpiperazin-1-yl)-8-methyl-9-(((S)-tetrahydrofuran-2-yl)methyl)-9H-purine (Step 1), cesium carbonate (3.27 g, 10.03 mmol), and methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (0.428 g, 0.501 mmol, Aldrich 745979) in 1,4-dioxane (4 mL) was added hydrazine (0.79 mL, 25 mmol), and the mixture was purged with nitrogen and stirred at 90° C. for 1 h. After cooling to rt, the reaction mixture was diluted with CH₂Cl₂ and filtered through a pad of MgSO₄ in a SiliaPrep SPE thiol cartridge (500 mg, SiliCycle SPE-R51030B-06P). The filtrate was concentrated, and the crude residue was purified by flash column chromatography (40 g SiO₂, 0-5% MeOH/CH₂Cl₂) to afford the desired product (1.50 g, 49% yield over 2 steps) as a mixture of diastereomers in the form of an off-white solid. LC-MS calculated for C₂₉H₃₈F₅N₈O (M+H)⁺: m/z=609.3. found 609.4.

### Step 3. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine

To a mixture of 6-((2S,5R)-4-((3,3-difluorocyclobutyl)(4-(trifluoromethyl)phenyl)methyl)-2,5-dimethylpiperazin-1-yl)-2-hydrazineyl-8-methyl-9-(((S)-tetrahydrofuran-2-yl)methyl)-9H-purine (1.50 g, 2.46 mmol) in AcOH (2.87 mL, 50.1 mmol) was added triethyl orthoformate (1.85 mL, 11.1 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₃₀H₃₆F₅N₈O (M+H)⁺: m/z=619.3. found 619.3. ¹H NMR (500 MHz, DMSO-d₆) (mixture of rotamers) δ 9.48 (s, 1H), 7.81-7.70 (m, 2H), 7.68-7.56 (m, 2H), 6.20-5.99 (m, 0.4H), 5.90-5.64 (m, 0.6H), 5.06-4.80 (m, 0.6H), 4.79-4.64 (m, 1H), 4.62-4.40 (m, 1.4H), 4.18-4.04 (m, 1H), 3.81-3.64 (m, 2H), 3.64-3.46 (m, 1.6H), 3.46-3.27 (m, 0.4H), 3.16-2.97 (m, 1H), 2.95-2.74 (m, 2H), 2.74-2.52 (m, 5H), 2.47-2.31 (m, 1H), 2.29-2.09 (m, 2H), 2.09-1.98 (m, 1H), 1.98-1.88 (m, 1H), 1.88-1.77 (m, 1H), 1.77-1.66 (m, 1H), 1.58-1.27 (m, 3H), 1.09-0.81 (m, 3H).

### Example 3. 4-((2S,5R)-4-((3-Chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

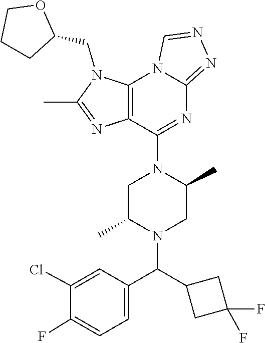 [SMILES: Cc1nc2c(N3C[C@@H](C)N(C(c4ccc(F)c(Cl)c4)C4CC(F)(F)C4)C[C@@H]3C)nc3nncn3c2n1C[C@@H]1CCCO1]

### Step 1. 2-Chloro-6-((2S,5R)-4-((3-chloro-4-fluorophenyl)(3,3-difluorocyclobutyl)methyl)-2,5-dimethylpiperazin-1-yl)-8-methyl-9-(((S)-tetrahydrofuran-2-yl)methyl)-9H-purine

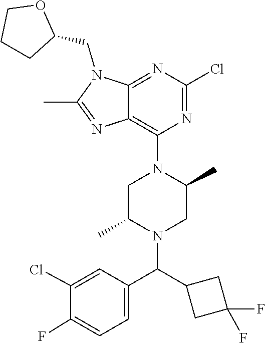 [SMILES: Cc1nc2c(N3C[C@@H](C)N(C(c4ccc(F)c(Cl)c4)C4CC(F)(F)C4)C[C@@H]3C)nc(Cl)nc2n1C[C@@H]1CCCO1]

To a mixture of (S)-2,6-dichloro-8-methyl-9-((tetrahydrofuran-2-yl)methyl)-9H-purine (Intermediate 5, 0.90 g, 3.13 mmol) and (2R,5S)-1-((3-chloro-4-fluorophenyl)(3,3-difluorocyclobutyl)methyl)-2,5-dimethylpiperazine hydrochloride (Intermediate 44, 1.20 g, 3.13 mmol) in n-BuOH (4 mL) was added N,N-diisopropylethylamine (1.64 mL, 9.39 mmol) and the mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₈H₃₄Cl₂F₃N₆O (M+H)⁺: m/z=597.2. found 597.1.

### Step 2. 6-((2S,5R)-4-((3-Chloro-4-fluorophenyl)(3,3-difluorocyclobutyl)methyl)-2,5-dimethylpiperazin-1-yl)-2-hydrazineyl-8-methyl-9-(((S)-tetrahydrofuran-2-yl)methyl)-9H-purine

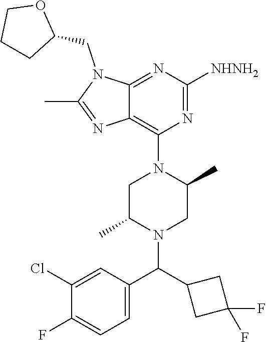 [SMILES: Cc1nc2c(N3C[C@@H](C)N(C(c4ccc(F)c(Cl)c4)C4CC(F)(F)C4)C[C@@H]3C)nc(NN)nc2n1C[C@@H]1CCCO1]

To a mixture of 2-chloro-6-((2S,5R)-4-((3-chloro-4-fluorophenyl)(3,3-difluorocyclobutyl)methyl)-2,5-dimethylpiperazin-1-yl)-8-methyl-9-(((S)-tetrahydrofuran-2-yl)methyl)-9H-purine (Step 1) in 1,4-dioxane (4 mL) was added hydrazine (0.49 mL, 16 mmol), and the mixture was stirred at 120° C. overnight. After cooling to rt, the reaction mixture was concentrated, and the crude residue was purified by flash column chromatography (40 g SiO₂, 0-5% MeOH/CH₂Cl₂) to afford the desired product (810 mg, 44% yield over 2 steps) as a mixture of diastereomers in the form of an off-white solid. LC-MS calculated for C₂₈H₃₇ClF₃N₈O (M+H)⁺: m/z=593.3. found 593.4.

### Step 3. 4-((2S,5R)-4-((3-Chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

To a mixture of 6-((2S,5R)-4-((3-chloro-4-fluorophenyl)(3,3-difluorocyclobutyl)methyl)-2,5-dimethylpiperazin-1-yl)-2-hydrazineyl-8-methyl-9-(((S)-tetrahydrofuran-2-yl)methyl)-9H-purine (810 mg, 1.37 mmol) in AcOH (1.79 mL, 31.3 mmol) was added triethyl orthoformate (1.15 mL, 6.91 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₂₉H₃₅ClF₃N₈O (M+H)⁺: m/z=603.3. found 603.8. ¹H NMR (600 MHz, DMSO-d₆) (mixture of rotamers) δ 9.49 (s, 1H), 7.73-7.53 (m, 1H), 7.52-7.32 (m, 2H), 6.19-5.95 (m, 0.4H), 5.89-5.69 (m, 0.6H), 5.00-4.81 (m, 0.6H), 4.79-4.65 (m, 1H), 4.64-4.44 (m, 1.4H), 4.16-4.06 (m, 1H), 3.80-3.24 (m, 4H), 3.19-2.95 (m, 1H), 2.94-2.74 (m, 2H), 2.73-2.52 (m, 5H), 2.45-2.29 (m, 1H), 2.28-2.09 (m, 2H), 2.08-1.98 (m, 1H), 1.98-1.89 (m, 1H), 1.88-1.78 (m, 1H), 1.77-1.68 (m, 1H), 1.59-1.27 (m, 3H), 1.10-0.84 (m, 3H).

### Example 4. 4-((2S,5R)-4-((4-Chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

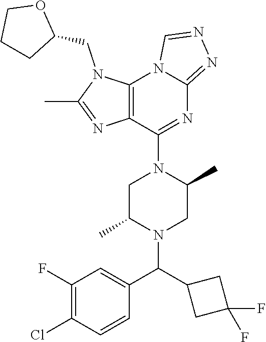 [SMILES: Cc1nc2c(N3C[C@@H](C)N(C(c4ccc(Cl)c(F)c4)C4CC(F)(F)C4)C[C@@H]3C)nc3nncn3c2n1C[C@@H]1CCCO1]

### Step 1. 2-Chloro-6-((2S,5R)-4-((4-chloro-3-fluorophenyl)(3,3-difluorocyclobutyl)methyl)-2,5-dimethylpiperazin-1-yl)-8-methyl-9-(((S)-tetrahydrofuran-2-yl)methyl)-9H-purine

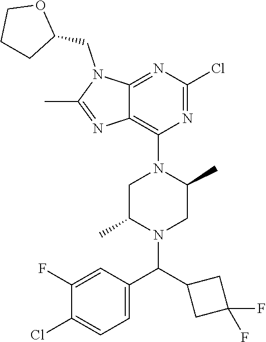 [SMILES: Cc1nc2c(N3C[C@@H](C)N(C(c4ccc(Cl)c(F)c4)C4CC(F)(F)C4)C[C@@H]3C)nc(Cl)nc2n1C[C@@H]1CCCO1]

To a mixture of (S)-2,6-dichloro-8-methyl-9-((tetrahydrofuran-2-yl)methyl)-9H-purine (Intermediate 5, 75 mg, 0.26 mmol) and (2R,5S)-1-((4-chloro-3-fluorophenyl)(3,3-difluorocyclobutyl)methyl)-2,5-dimethylpiperazine hydrochloride (Intermediate 48, 100 mg, 0.26 mmol) in n-BuOH (4 mL) was added N,N-diisopropylethylamine (0.14 mL, 0.8 mmol) and the mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated in vacuo to afford the desired product as a mixture of diastereomers. The crude material obtained was used directly without further purification. LC-MS calculated for C₂₈H₃₄Cl₂F₃N₆O (M+H)⁺: m/z=597.2. found 597.3.

### Step 2. 6-((2S,5R)-4-((4-Chloro-3-fluorophenyl)(3,3-difluorocyclobutyl)methyl)-2,5-dimethylpiperazin-1-yl)-2-hydrazineyl-8-methyl-9-(((S)-tetrahydrofuran-2-yl)methyl)-9H-purine

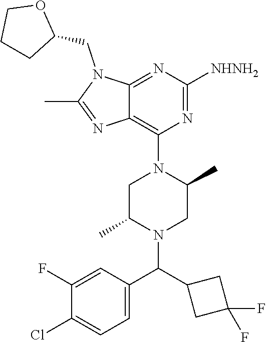 [SMILES: Cc1nc2c(N3C[C@@H](C)N(C(c4ccc(Cl)c(F)c4)C4CC(F)(F)C4)C[C@@H]3C)nc(NN)nc2n1C[C@@H]1CCCO1]

To a mixture of 2-chloro-6-((2S,5R)-4-((4-chloro-3-fluorophenyl)(3,3-difluorocyclobutyl)methyl)-2,5-dimethylpiperazin-1-yl)-8-methyl-9-(((S)-tetrahydrofuran-2-yl)methyl)-9H-purine (Step 1) in 1,4-dioxane (4 mL) was added hydrazine (41 μL, 1.3 mmol), and the mixture was stirred at 120° C. overnight. After cooling to rt, the reaction mixture was concentrated, and the crude residue was purified by flash column chromatography (SiO₂, 0-5% MeOH/CH₂Cl₂) to afford the desired product (37 mg, 24% yield over 2 steps) as a mixture of diastereomers in the form of an off-white solid. LC-MS calculated for C₂₈H₃₇ClF₃N₈O (M+H)⁺: m/z=593.3. found 593.4.

### Step 3. 4-((2S,5R)-4-((4-Chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine

To a mixture of 6-((2S,5R)-4-((4-chloro-3-fluorophenyl)(3,3-difluorocyclobutyl)methyl)-2,5-dimethylpiperazin-1-yl)-2-hydrazineyl-8-methyl-9-(((S)-tetrahydrofuran-2-yl)methyl)-9H-purine (37 mg, 0.062 mmol) in AcOH (0.15 mL, 2.6 mmol) was added triethyl orthoformate (0.1 mL, 0.6 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₂₉H₃₅ClF₃N₈O (M+H)⁺: m/z=603.3. found 603.4. ¹H{¹⁹F} NMR (500 MHz, DMSO-d₆) (mixture of rotamers) δ 9.47 (s, 1H), 7.60 (d, J=8.2 Hz, 1H), 7.46 (s, 1H), 7.26 (d, J=8.2 Hz, 1H), 6.19-5.97 (m, 0.4H), 5.88-5.65 (m, 0.6H), 5.00-4.81 (m, 0.6H), 4.80-4.64 (m, 1H), 4.63-4.41 (m, 1.4H), 4.16-4.05 (m, 1H), 3.74-3.51 (m, 3.6H), 3.45-3.32 (m, 0.4H), 3.09-2.97 (m, 1H), 2.91-2.74 (m, 2H), 2.73-2.53 (m, 5H), 2.42-2.31 (m, 1H), 2.28-2.09 (m, 2H), 2.08-2.01 (m, 1H), 1.97-1.89 (m, 1H), 1.88-1.77 (m, 1H), 1.77-1.66 (m, 1H), 1.54-1.29 (m, 3H), 1.00-0.87 (m, 3H).

### Example 5. 4-((2S,5R)-4-(Bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine

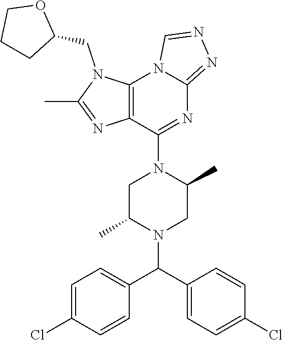 [SMILES: Cc1nc2c(N3C[C@@H](C)N(C(c4ccc(Cl)cc4)c4ccc(Cl)cc4)C[C@@H]3C)nc3nncn3c2n1C[C@@H]1CCCO1]

### Step 1: 6-((2S,5R)-4-(Bis(4-chlorophenyl)methyl)-2,5-dimethylpiperazin-1-yl)-2-chloro-8-methyl-9-(((S)-tetrahydrofuran-2-yl)methyl)-9H-purine

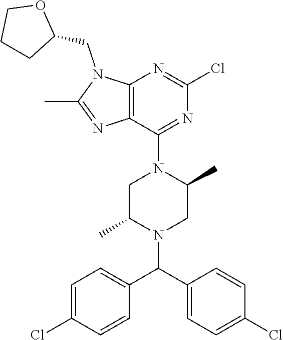 [SMILES: Cc1nc2c(N3C[C@@H](C)N(C(c4ccc(Cl)cc4)c4ccc(Cl)cc4)C[C@@H]3C)nc(Cl)nc2n1C[C@@H]1CCCO1]

A mixture of (2R,5S)-1-(bis(4-chlorophenyl)methyl)-2,5-dimethylpiperazine hydrochloride (Intermediate 83, 0.463 g, 1.20 mmol), (S)-2,6-dichloro-8-methyl-9-((tetrahydrofuran-2-yl)methyl)-9H-purine (Intermediate 5, 0.345 g, 1.20 mmol), and N,N-diisopropylethylamine (0.629 mL, 3.60 mmol) in n-BuOH (3.0 mL) was heated to 85° C. and stirred overnight. After cooling to rt, the mixture was diluted with CH₂Cl₂, water, and 1 M NaOH. The layers were separated and the aqueous layer was extracted with CH₂Cl₂. The combined organic layers were dried over MgSO₄, concentrated in vacuo, and the crude residue was purified by flash column chromatography (24 g SiO₂, EtOAc/hexanes) to give the title compound (0.552 g, 77% yield) as an orange solid. LC-MS calculated for C₃₀H₃₄Cl₃N₆O (M+H)⁺: m/z=599.2. found 599.2.

### Step 2: 6-((2S,5R)-4-(Bis(4-chlorophenyl)methyl)-2,5-dimethylpiperazin-1-yl)-2-hydrazineyl-8-methyl-9-(((S)-tetrahydrofuran-2-yl)methyl)-9H-purine

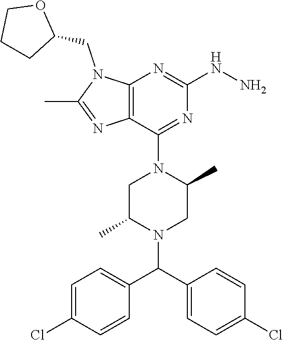 [SMILES: Cc1nc2c(N3C[C@@H](C)N(C(c4ccc(Cl)cc4)c4ccc(Cl)cc4)C[C@@H]3C)nc(NN)nc2n1C[C@@H]1CCCO1]

A mixture of 6-((2S,5R)-4-(bis(4-chlorophenyl)methyl)-2,5-dimethylpiperazin-1-yl)-2-chloro-8-methyl-9-(((S)-tetrahydrofuran-2-yl)methyl)-9H-purine (0.552 g, 0.920 mmol) and hydrazine hydrate (1.15 mL, 18.5 mmol) in n-BuOH (3.45 mL) was heated to 120° C. and stirred for 20 h. After cooling to rt, the mixture was diluted with CH₂Cl₂, water, and 1 M NaOH. The layers were separated and the aqueous layer was extracted with CH₂Cl₂. The combined organic layers were dried over MgSO₄, concentrated in vacuo, and the crude residue was purified by flash column chromatography (24 g SiO₂, MeOH/CH₂Cl₂) to give the title compound (0.484 g, 88% yield) as a white solid. LC-MS calculated for C₃₀H₃₇Cl₂N₈O (M+H)⁺: m/z=595.3. found 595.2.

### Step 3: 4-((2S,5R)-4-(Bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine

A mixture of 6-((2S,5R)-4-(bis(4-chlorophenyl)methyl)-2,5-dimethylpiperazin-1-yl)-2-hydrazineyl-8-methyl-9-(((S)-tetrahydrofuran-2-yl)methyl)-9H-purine (0.484 g, 0.813 mmol), acetic acid (2.33 mL, 40.7 mmol) and triethyl orthoformate (0.67 mL, 4.07 mmol) was stirred at 95° C. for 1 h. The mixture was cooled to rt, diluted with CH₂Cl₂ and slowly transferred to a separatory funnel containing saturated aqueous NaHCO₃. Following transfer, the aqueous layer was further basified with 1 M NaOH to pH=10. The layers were separated, and the aqueous layer extracted with CH₂Cl₂. The combined organic layers were dried over MgSO₄ and concentrated in vacuo. The crude residue was purified by flash column chromatography (24 g SiO₂, MeOH/CH₂Cl₂) to give the title compound (0.288 g, 58% yield) as a light orange solid. The material was further purified by prep-HPLC (Sunfire C18 column, eluting with a gradient of acetonitrile/water containing 0.1% TFA, at flow rate of 60 mL/min) to afford the title compound as its TFA salt. LC-MS calculated for C₃₁H₃₅Cl₂N₈O (M+H)⁺: m/z=605.2. found 605.2. ¹H NMR (500 MHz, DMSO-d₆) (mixture of rotamers) δ 9.52-9.48 (m, 1H), 7.64-7.55 (m, 4H), 7.43-7.37 (m, 4H), 6.25-6.18 (m, 0.4H), 5.96-5.91 (m, 0.6H), 5.11-5.04 (m, 0.6H), 4.76-4.67 (m, 2H), 4.66-4.61 (m, 0.4H), 4.59-4.51 (m, 1H), 4.14-4.06 (m, 1H), 3.89-3.83 (m, 0.6H), 3.73-3.67 (m, 1H), 3.67-3.61 (m, 0.4H), 3.59-3.52 (m, 1H), 3.24-3.19 (m, 0.4H), 3.18-3.14 (m, 0.6H), 2.83-2.77 (m, 0.4H), 2.76-2.69 (m, 0.6H), 2.59 (s, 1.2H), 2.57 (s, 1.8H), 2.50-2.46 (m, 0.6H), 2.45-2.41 (m, 0.4H), 2.17-2.08 (m, 1H), 1.99-1.89 (m, 1H), 1.88-1.78 (m, 1H), 1.77-1.67 (m, 1H), 1.53 (d, J=6.6 Hz, 1.2H), 1.48 (d, J=6.8 Hz, 1.8H), 0.93-0.88 (m, 3H).

### Example 6. 4-((2S,5R)-4-((3,3-Difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine

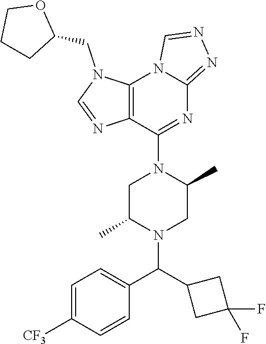 [SMILES: C[C@@H]1CN(c2nc3nncn3c3c2ncn3C[C@@H]2CCCO2)[C@@H](C)CN1C(c1ccc(C(F)(F)F)cc1)C1CC(F)(F)C1]

### Step 1. 2-Chloro-6-((2S,5R)-4-((3,3-difluorocyclobutyl)(4-(trifluoromethyl)phenyl)methyl)-2,5-dimethylpiperazin-1-yl)-9-(((S)-tetrahydrofuran-2-yl)methyl)-9H-purine

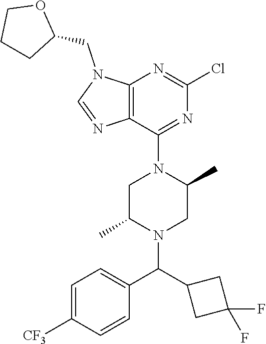 [SMILES: C[C@@H]1CN(c2nc(Cl)nc3c2ncn3C[C@@H]2CCCO2)[C@@H](C)CN1C(c1ccc(C(F)(F)F)cc1)C1CC(F)(F)C1]

To a mixture of (S)-2,6-dichloro-9-((tetrahydrofuran-2-yl)methyl)-9H-purine (Intermediate 1, 0.100 g, 0.366 mmol) and (2R,5S)-1-((3,3-difluorocyclobutyl)(4-(trifluoromethyl)phenyl)methyl)-2,5-dimethylpiperazine hydrochloride (Intermediate 3, 0.146 g, 0.366 mmol) in 1-butanol (4 mL) was added N,N-diisopropylethylamine (0.192 mL, 1.10 mmol) and the mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₂₈H₃₃ClF₅N₆O (M+H)⁺: m/z=599.2. found 599.4.

### Step 2. 6-((2S,5R)-4-((3,3-Difluorocyclobutyl)(4-(trifluoromethyl)phenyl)methyl)-2,5-dimethylpiperazin-1-yl)-2-hydrazineyl-9-(((S)-tetrahydrofuran-2-yl)methyl)-9H-purine

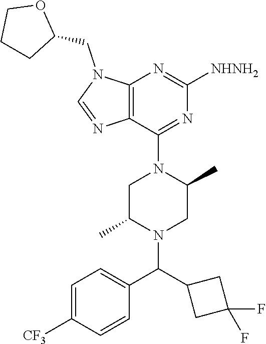 [SMILES: C[C@@H]1CN(c2nc(NN)nc3c2ncn3C[C@@H]2CCCO2)[C@@H](C)CN1C(c1ccc(C(F)(F)F)cc1)C1CC(F)(F)C1]

To a mixture of 2-chloro-6-((2S,5R)-4-((3,3-difluorocyclobutyl)(4-(trifluoromethyl)phenyl)methyl)-2,5-dimethylpiperazin-1-yl)-9-(((S)-tetrahydrofuran-2-yl)methyl)-9H-purine (Step 1) in 1,4-dioxane (4 mL) was added hydrazine (0.057 mL, 1.83 mmol), and the mixture was stirred at 120° C. for 2 h. After cooling to rt, the reaction mixture was concentrated, and the crude residue was purified by flash column chromatography (12 g SiO₂, 0-5% MeOH/CH₂Cl₂) to afford the desired product (84.4 mg, 39% yield over 2 steps) as a mixture of diastereomers in the form of an off-white solid. LC-MS calculated for C₂₈H₃₆F₅N₈O (M+H)⁺: m/z=595.3. found 595.5.

### Step 3. 4-((2S,5R)-4-((3,3-Difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine

To a mixture of 6-((2S,5R)-4-((3,3-difluorocyclobutyl)(4-(trifluoromethyl)phenyl)methyl)-2,5-dimethylpiperazin-1-yl)-2-hydrazineyl-9-(((S)-tetrahydrofuran-2-yl)methyl)-9H-purine (84.4 mg, 0.142 mmol) in AcOH (2.10 mL, 36.6 mmol) was added triethyl orthoformate (0.122 mL, 0.732 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₂₉H₃₄F₅N₈O (M+H)⁺: m/z=605.3. found 605.3. ¹H NMR (600 MHz, DMSO-d₆) (mixture of rotamers) δ 9.54 (s, 1H), 8.47-8.25 (m, 1H), 7.87-7.73 (m, 2H), 7.70-7.56 (m, 2H), 6.25-6.00 (m, 0.4H), 5.90-5.57 (m, 0.6H), 5.05-4.86 (m, 0.6H), 4.85-4.73 (m, 1H), 4.65-4.45 (m, 1.4H), 4.24-4.10 (m, 1H), 3.88-3.70 (m, 1H), 3.70-3.59 (m, 1.6H), 3.59-3.52 (m, 1H), 3.44-3.30 (m, 0.4H), 3.23-3.00 (m, 1H), 2.92-2.75 (m, 2H), 2.75-2.55 (m, 2H), 2.46-2.31 (m, 1H), 2.26-2.13 (m, 1H), 2.11-1.94 (m, 2H), 1.85-1.76 (m, 1H), 1.76-1.62 (m, 2H), 1.55-1.27 (m, 3H), 1.08-0.81 (m, 3H).

### Example 7. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

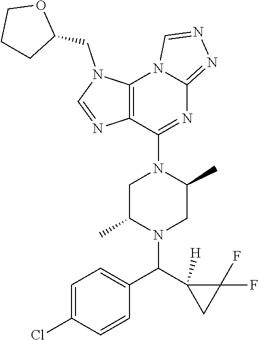 [SMILES: C[C@@H]1CN(c2nc3nncn3c3c2ncn3C[C@@H]2CCCO2)[C@@H](C)CN1C(c1ccc(Cl)cc1)[C@@H]1CC1(F)F]

### Step 1: 2-Chloro-6-((2S,5R)-4-((4-chlorophenyl)((S)-2,2-difluorocyclopropyl)methyl)-2,5-dimethylpiperazin-1-yl)-9-(((S)-tetrahydrofuran-2-yl)methyl)-9H-purine

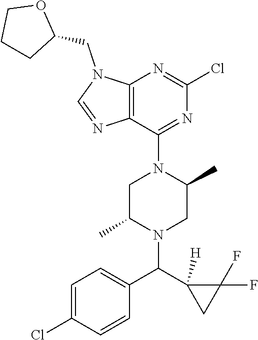 [SMILES: C[C@@H]1CN(c2nc(Cl)nc3c2ncn3C[C@@H]2CCCO2)[C@@H](C)CN1C(c1ccc(Cl)cc1)[C@@H]1CC1(F)F]

A mixture of (S)-2,6-dichloro-9-((tetrahydrofuran-2-yl)methyl)-9H-purine (Intermediate 1, 0.389 g, 1.42 mmol), (2R,5S)-1-((4-chlorophenyl)((S)-2,2-difluorocyclopropyl)methyl)-2,5-dimethylpiperazine hydrochloride (Intermediate 16, 0.500 g, 1.42 mmol), and N,N-diisopropylethylamine (0.746 mL, 4.27 mmol) in 2-propanol (3.56 mL) was stirred at 85° C. overnight. The mixture was cooled to room temperature and concentrated in vacuo. The residue was diluted with CH₂Cl₂ and quenched with water and saturated aqueous NaHCO₃. The layers were separated and the aqueous layer was extracted with CH₂Cl₂. The combined organic layers were dried over MgSO₄, concentrated in vacuo, and purified by flash column chromatography (24 g SiO₂, EtOAc/hexanes) to give the title compound (0.605 g, 1.10 mmol, 77% yield) as an off-white solid. LC-MS calculated for C₂₆H₃₁Cl₂F₂N₆O (M+H)⁺: m/z=551.2. found 551.2.

### Step 2: 6-((2S,5R)-4-((4-Chlorophenyl)((S)-2,2-difluorocyclopropyl)methyl)-2,5-dimethylpiperazin-1-yl)-2-hydrazineyl-9-(((S)-tetrahydrofuran-2-yl)methyl)-9H-purine

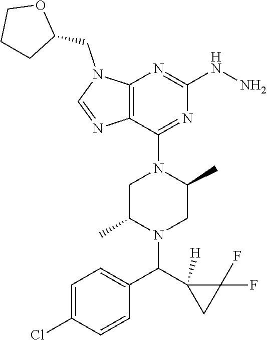 [SMILES: C[C@@H]1CN(c2nc(NN)nc3c2ncn3C[C@@H]2CCCO2)[C@@H](C)CN1C(c1ccc(Cl)cc1)[C@@H]1CC1(F)F]

A mixture of 2-chloro-6-((2S,5R)-4-((4-chlorophenyl)((S)-2,2-difluorocyclopropyl)methyl)-2,5-dimethylpiperazin-1-yl)-9-(((S)-tetrahydrofuran-2-yl)methyl)-9H-purine (Step 1) and hydrazine hydrate (0.683 mL, 10.96 mmol, Aldrich 225819) in n-BuOH (3.56 mL) was stirred at 120° C. overnight. The mixture was cooled to room temperature, diluted with CH₂Cl₂, and quenched with saturated aqueous NaHCO₃. The layers were separated and the aqueous layer was extracted with CH₂C₂. The combined organic layers were dried over MgSO₄, concentrated in vacuo, and the residue was purified by flash column chromatography (24 g SiO₂, MeOH/CH₂Cl₂) to give the title compound (0.542 g, 0.991 mmol, 70% yield) as a white solid. LC-MS calculated for C₂₆H₃₄ClF₂N₈O (M+H)⁺: m/z=547.3. found 547.3.

### Step 3: 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

A mixture of 6-((2S,5R)-4-((4-chlorophenyl)((S)-2,2-difluorocyclopropyl)methyl)-2,5-dimethylpiperazin-1-yl)-2-hydrazineyl-9-(((S)-tetrahydrofuran-2-yl)methyl)-9H-purine (Step 2) and triethyl orthoformate (0.415 mL, 2.49 mmol) in acetic acid (1.43 mL, 24.91 mmol) was stirred at 95° C. for 1 h. The mixture was concentrated in vacuo and the residue was diluted with CH₂Cl₂ and carefully quenched with saturated aqueous NaHCO₃. The layers were separated and the aqueous layer was extracted with CH₂Cl₂. The combined organic layers were dried over MgSO₄ and concentrated in vacuo. The residue was purified by flash column chromatography (24 g SiO₂, MeOH/CH₂Cl₂) to give the title compound (0.279 g, 35% yield) as a light yellow solid. The material was taken up in CH₂Cl₂ (2 mL) and Et₂O (10 mL) was added slowly. The precipitate was collected via filtration, washing with 1:1 diethyl ether/hexanes (20 mL). The solid was dried under vacuum on the filter for 30 minutes, then transferred to a vial and dried under high vacuum at 40° C. overnight. The solid material was taken up in 1:1 MeCN/H₂O (10 mL) and TFA (0.173 mL, 2.25 mmol) was added. The mixture was frozen and lyophilized to give the final product as its TFA salt. Further purification by prep-HPLC (Sunfire C18 column, eluting with a gradient of acetonitrile/water containing 0.1% TFA, at flow rate of 60 mL/min) afforded the title compound as a single stereoisomer as its TFA salt. LC-MS calculated for C₂₇H₃₂ClF₂N₈O (M+H)⁺: m/z=557.2. found 557.3. ¹H NMR (500 MHz, DMSO-d₆) (mixture of rotamers) δ 9.54 (s, 1H), 8.36 (s, 1H), 7.52-7.42 (m, 4H), 6.14-5.88 (m, 1H), 4.95-4.92 (m, 0.6H), 4.84-4.77 (m, 1.4H), 4.61-4.53 (m, 1H), 4.23-4.14 (m, 1H), 3.93-3.87 (m, 0.6H), 3.67-3.64 (m, 2.4H), 3.60-3.52 (m, 1H), 3.39-3.26 (m, 1H), 2.75-2.71 (m, 1H), 2.29-2.25 (m, 1H), 2.19-2.02 (m, 2H), 1.86-1.65 (m, 3H), 1.57-1.46 (m, 1H), 1.39-1.25 (m, 3H), 1.07-0.97 (m, 4H).

### Example 8. 4-((2S,5R)-4-((4-Chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine

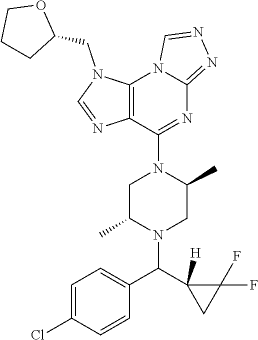 [SMILES: C[C@@H]1CN(c2nc3nncn3c3c2ncn3C[C@@H]2CCCO2)[C@@H](C)CN1C(c1ccc(Cl)cc1)[C@H]1CC1(F)F]

### Step 1: (2R,5S)-1-((4-Chlorophenyl)((R)-2,2-difluorocyclopropyl)methyl)-2,5-dimethylpiperazine hydrochloride

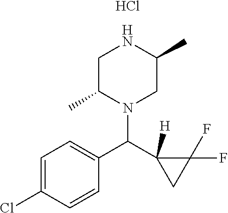 [SMILES: Cl ; C[C@@H]1CN[C@@H](C)CN1C(c1ccc(Cl)cc1)[C@H]1CC1(F)F]

A mixture of tert-butyl (2S,5R)-4-((4-chlorophenyl)((R)-2,2-difluorocyclopropyl)methyl)-2,5-dimethylpiperazine-1-carboxylate (Intermediate 19, 0.352 mmol) in THE (0.88 mL) was treated with HCl (4 M in 1,4-dioxane) (0.879 mL, 3.52 mmol) and stirred at 60° C. for 1 h. The mixture was cooled to room temperature and concentrated in vacuo and the product was used without further purification. LC-MS calculated for C₁₆H₂₂ClF₂N₂(M+H)⁺: m/z=315.1. found 315.1.

### Step 2: 2-Chloro-6-((2S,5R)-4-((4-chlorophenyl)((R)-2,2-difluorocyclopropyl)methyl)-2,5-dimethylpiperazin-1-yl)-9-(((S)-tetrahydrofuran-2-yl)methyl)-9H-purine

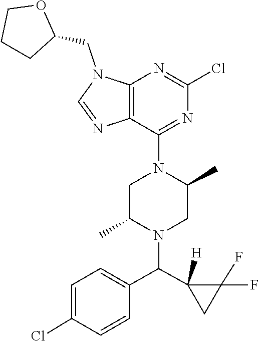 [SMILES: C[C@@H]1CN(c2nc(Cl)nc3c2ncn3C[C@@H]2CCCO2)[C@@H](C)CN1C(c1ccc(Cl)cc1)[C@H]1CC1(F)F]

A mixture of (2R,5S)-1-((4-chlorophenyl)((R)-2,2-difluorocyclopropyl)methyl)-2,5-dimethylpiperazine hydrochloride (Step 1, 0.176 mmol) and (S)-2,6-dichloro-9-((tetrahydrofuran-2-yl)methyl)-9H-purine (Intermediate 1, 48.0 mg, 0.176 mmol), and N,N-diisopropylethylamine (0.154 mL, 0.879 mmol) in 2-propanol (0.44 mL) was stirred at 90° C. overnight. The mixture cooled to room temperature, concentrated in vacuo, and directly purified by flash column chromatography (12 g SiO₂, EtOAc/hexanes) to afford 15 the major diastereomer of the title compound as a single stereoisomer. LC-MS calculated for C₂₆H₃₁Cl₂F₂N₆O (M+H)⁺: m/z=551.2. found 551.3.

### Step 3: 6-((2S,5R)-4-((4-Chlorophenyl)((R)-2,2-difluorocyclopropyl)methyl)-2,5-dimethylpiperazin-1-yl)-2-hydrazineyl-9-(((S)-tetrahydrofuran-2-yl)methyl)-9H-purine

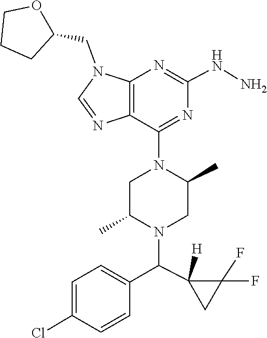 [SMILES: C[C@@H]1CN(c2nc(NN)nc3c2ncn3C[C@@H]2CCCO2)[C@@H](C)CN1C(c1ccc(Cl)cc1)[C@H]1CC1(F)F]

A mixture of 2-chloro-6-((2S,5R)-4-((4-chlorophenyl)((R)-2,2-difluorocyclopropyl)methyl)-2,5-dimethylpiperazin-1-yl)-9-(((S)-tetrahydrofuran-2-yl)methyl)-9H-purine (Step 2, 72.5 μmol) and hydrazine hydrate (45.2 μL, 0.725 mmol, Aldrich 225819) in n-BuOH (0.18 mL) was stirred at 120° C. overnight. The mixture was cooled to room temperature, concentrated in vacuo, and the residue was directly purified by flash column chromatography (12 g SiO₂, MeOH/CH₂Cl₂) to give the title compound as a clear oil. LC-MS calculated for C₂₆H₃₄ClF₂N₈O (M+H)⁺: m/z=547.3. found 547.3.

### Step 4: 4-((2S,5R)-4-((4-Chlorophenyl)((R)-2,2-di fluorocyclopropyl)methyl)-2,5-dimethylpiperazin-1-yl)-1-(((S)-tetrahydrofuran-2-yl)methyl)-1H-[1,2,4]triazolo[3,4-b]purine

A mixture of 6-((2S,5R)-4-((4-chlorophenyl)((R)-2,2-difluorocyclopropyl)methyl)-2,5-dimethylpiperazin-1-yl)-2-hydrazineyl-9-(((S)-tetrahydrofuran-2-yl)methyl)-9H-purine (Step 3) and triethyl orthoformate (27.8 μL, 0.167 mmol) in acetic acid (96.0 μL, 1.67 mmol) was stirred at 95° C. for 1 h. The mixture was cooled to room temperature, diluted with MeOH, and purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₇H₃₂ClF₂N₈O (M+H)⁺: m/z=557.2. found 557.3. ¹H NMR (500 MHz, DMSO-d₆) (mixture of rotamers) δ 9.57 (s, 1H), 8.40-8.35 (m, 1H), 7.51 (d, J=8.8 Hz, 2H), 7.46 (d, J=8.1 Hz, 2H), 6.10-5.96 (m, 1H), 4.97-4.93 (m, 0.5H), 4.85-4.79 (m, 1.5H), 4.59 (dd, J=15.1, 7.7 Hz, 1H), 4.19 (qd, J=7.1, 2.8 Hz, 1H), 3.98-3.88 (m, 0.5H), 3.70-3.62 (m, 2.5H), 3.60-3.53 (m, 1H), 3.48-3.32 (m, 1H), 2.85-2.62 (m, 1H), 2.39-2.01 (m, 3H), 1.86-1.64 (m, 3H), 1.60-1.50 (m, 1H), 1.37-1.24 (m, 3H), 1.08-0.97 (m, 4H).

### Example 9. 4-((2S,5R)-4-((4-Chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine

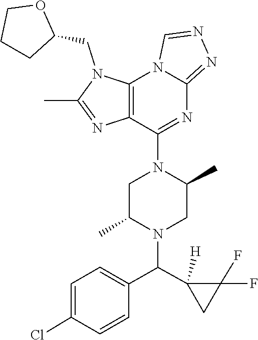 [SMILES: Cc1nc2c(N3C[C@@H](C)N(C(c4ccc(Cl)cc4)[C@@H]4CC4(F)F)C[C@@H]3C)nc3nncn3c2n1C[C@@H]1CCCO1]

This compound was prepared according to the procedures outlined in Steps 2-4 for Example 8, with (2R,5S)-1-((4-chlorophenyl)((S)-2,2-difluorocyclopropyl)methyl)-2,5-dimethylpiperazine hydrochloride (Intermediate 16) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The major diastereomer of the title compound was isolated as a single stereoisomer as its TFA salt. LC-MS calculated for C₂₈H₃₄ClF₂N₈O (M+H)⁺: m/z=571.3. found 571.3. ¹H NMR (500 MHz, DMSO-d₆) (mixture of rotamers) δ 9.52 (s, 1H), 7.59-7.54 (m, 2H), 7.49-7.45 (m, 2H), 6.44-6.40 (m, 0.5H), 5.91-5.79 (m, 0.5H), 5.23-5.20 (m, 0.5H), 4.78-4.69 (m, 1H), 4.61-4.52 (m, 1.5H), 4.16-4.08 (m, 1H), 3.76-3.64 (m, 1.5H), 3.60-3.53 (m, 1H), 3.48-3.41 (m, 0.5H), 3.29-3.24 (m, 1H), 3.08-3.00 (m, 1H), 2.94-2.87 (m, 2H), 2.65 (s, 1.5H), 2.56 (s, 1.5H), 2.17-2.12 (m, 1H), 2.07-1.91 (m, 2H), 1.89-1.79 (m, 1H), 1.78-1.68 (m, 1H), 1.57 (d, J=6.5 Hz, 1.5H), 1.51-1.46 (m, 2.5H), 1.13-1.04 (m, 1H), 0.92-0.84 (m, 3H).

### Example 10. 4-((2S,5R)-4-(1-(4-Chlorophenyl)-3-methylbutyl)-2,5-dimethylpiperazin-1-yl)-1-(((S)-tetrahydrofuran-2-yl)methyl)-1H-[1,2,4]triazolo[3,4-b]purine

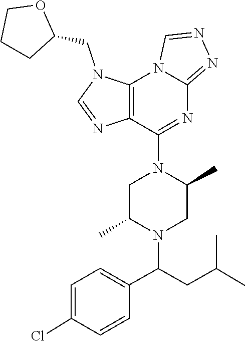

This compound was prepared according to the procedures outlined in Example 7, with (2S,5R)-4-(1-(4-chlorophenyl)-3-methylbutyl)-2,5-dimethylpiperazine hydrochloride (Intermediate 10) replacing (2R,5S)-1-((4-chlorophenyl)((S)-2,2-difluorocyclopropyl)methyl)-2,5-dimethylpiperazine hydrochloride. Following flash column chromatography in Step 3, the material obtained was purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₈H₃₈ClN₈O (M+H)⁺: m/z=537.3. found 537.3. ¹H NMR (500 MHz, DMSO-d₆) δ 9.47 (s, 1H), 8.31 (s, 1H), 7.60-7.37 (m, 4H), 5.81-4.95 (m, 2H), 4.80 (dd, J=15.0, 3.0 Hz, 1H), 4.58 (dd, J=15.0, 7.5 Hz, 1H), 4.22 (qd, J=7.1, 2.9 Hz, 1H), 4.03-3.80 (m, 1H), 3.74-3.51 (m, 3H), 3.40-2.76 (m, 3H), 2.11 (dtd, J=12.5, 7.3, 4.9 Hz, 1H), 1.90-1.66 (m, 5H), 1.42 (d, J=6.4 Hz, 3H), 1.28-1.19 (m, 1H), 1.12-1.02 (m, 3H), 0.88 (d, J=6.5 Hz, 3H), 0.82 (d, J=6.6 Hz, 3H).

### Example 11. 4-((2S,5R)-4-(1-(4-Chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine

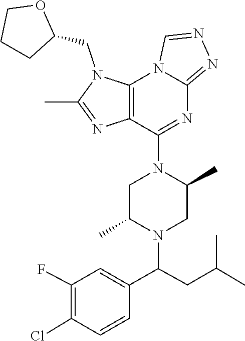

This compound was prepared according to the procedures outlined in Steps 2-4 for Example 8, with (2R,5S)-1-(1-(4-chloro-3-fluorophenyl)-3-methylbutyl)-2,5-dimethylpiperazine hydrochloride (Intermediate 12) replacing (2R,5S)-1-((4-chlorophenyl)((R)-2,2-difluorocyclopropyl)methyl)-2,5-dimethylpiperazine hydrochloride and (S)-2,6-dichloro-8-methyl-9-((tetrahydrofuran-2-yl)methyl)-9H-purine (Intermediate 5) replacing (S)-2,6-dichloro-9-((tetrahydrofuran-2-yl)methyl)-9H-purine. The title compound was isolated as a single stereoisomer as its TFA salt. LC-MS calculated for C₂₉H₃₉ClFN₈O (M+H)⁺: m/z=569.3. found 569.2.

### Example 12. 4-((2S,5R)-4-((4-Chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine

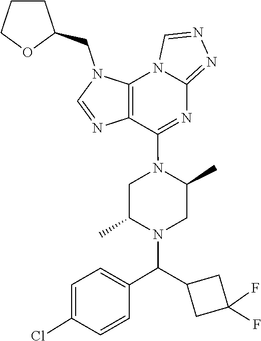

This compound was prepare according to the procedures described in Example 6, with (2R,5S)-1-((4-chlorophenyl)(3,3-difluorocyclobutyl)methyl)-2,5-dimethylpiperazine hydrochloride (Intermediate 39) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₈H₃₄ClF₂N₈O (M+H)⁺: m/z=571.3. found 571.4. ¹H NMR (500 MHz, DMSO-d₆) (mixture of rotamers) δ 9.54 (s, 1H), 8.48-8.24 (m, 1H), 7.66-7.28 (m, 4H), 6.18-5.99 (m, 0.4H), 5.96-5.57 (m, 0.6H), 5.04-4.88 (m, 0.6H), 4.88-4.74 (m, 1H), 4.65-4.47 (m, 1.4H), 4.24-4.08 (m, 2H), 3.71-3.61 (m, 1.6H), 3.61-3.51 (m, 1H), 3.45-3.27 (m, 0.4H), 3.22-3.00 (m, 1H), 2.97-2.74 (m, 2H), 2.74-2.54 (m, 2H), 2.47-2.32 (m, 1H), 2.29-2.16 (m, 1H), 2.14-1.96 (m, 2H), 1.89-1.77 (m, 1H), 1.77-1.66 (m, 2H), 1.54-1.27 (m, 3H), 1.09-0.84 (m, 3H).

### Example 13. 4-((2S,5R)-4-((4-Chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine

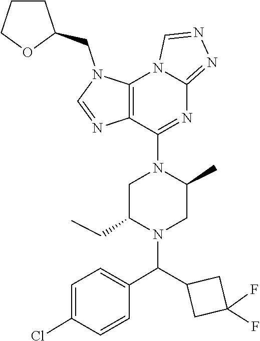 [SMILES: CC[C@@H]1CN(c2nc3nncn3c3c2ncn3C[C@@H]2CCCO2)[C@@H](C)CN1C(c1ccc(Cl)cc1)C1CC(F)(F)C1]

This compound was prepared according to the procedures described in Example 6, with (2R,5S)-1-((4-chlorophenyl)(3,3-difluorocyclobutyl)methyl)-2-ethyl-5-methylpiperazine hydrochloride (Intermediate 43) replacing (2R,5S)-1-((3,3-difluorocyclobutyl)(4-(trifluoromethyl)phenyl)methyl)-2,5-dimethylpiperazine hydrochloride in Step 1. LC-MS calculated for C₂₉H₃₆ClF₂N₈O (M+H)⁺: m/z=585.3; found 585.3. ¹H NMR (500 MHz, DMSO-d₆) (mixture of rotamers) δ 9.54 (s, 1H), 8.46-8.31 (m, 1H), 7.57-7.44 (m, 2H), 7.44-7.34 (m, 2H), 6.01-5.86 (m, 1H), 4.87-4.75 (m, 1.6H), 4.65-4.53 (m, 1.4H), 4.25-4.15 (m, 1H), 3.85-3.71 (m, 1H), 3.69-3.61 (m, 1H), 3.60-3.46 (m, 1.6H), 3.37-3.21 (m, 0.4H), 3.20-2.99 (m, 1H), 2.86-2.55 (m, 4H), 2.45-2.32 (m, 1H), 2.31-2.15 (m, 1H), 2.14-2.05 (m, 1H), 2.05-1.93 (m, 1H), 1.88-1.76 (m, 1H), 1.75-1.63 (m, 2H), 1.60-1.50 (m, 1H), 1.48-1.40 (m, 1H), 1.39-1.23 (m, 3H), 0.88-0.72 (m, 3H).

### Example 14. 4-((2S,5R)-4-(2-Fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine

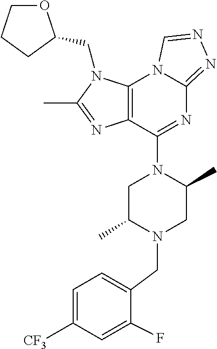

To a mixture of 4-((2S,5R)-2,5-dimethylpiperazin-1-yl)-2-methyl-1-(((S)-tetrahydrofuran-2-yl)methyl)-1H-[1,2,4]triazolo[3,4-b]purine hydrochloride (Intermediate 45, 10 mg, 0.025 mmol) in CH₂Cl₂ (0.5 mL) was added N,N-diisopropylethylamine (8.6 μL, 0.049 mmol) and 2-fluoro-4-(trifluoromethyl)benzaldehyde (9.4 mg, 0.049 mmol) and the reaction mixture was stirred at rt for 30 min. AcOH (2.1 μL, 0.037 mmol) was added and the reaction mixture was stirred at rt for 10 min before sodium triacetoxyborohydride (10.4 mg, 0.049 mmol) was added and the reaction mixture was stirred at rt for 4 h. The reaction mixture was diluted with methanol and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₂₆H₃₁F₄N₈O (M+H)⁺: m/z=547.3. found 547.3. ¹H NMR (600 MHz, DMSO-d₆, 70° C.) (mixture of rotamers) δ 9.45-9.42 (m, 1H), 7.84-7.78 (m, 1H), 7.64-7.59 (m, 2H), 5.83-5.23 (m, 2H), 4.77-4.70 (m, 1H), 4.61-4.52 (m, 1H), 4.17-4.10 (m, 1H), 3.87-3.81 (m, 1H), 3.76-3.68 (m, 3H), 3.62-3.55 (m, 1H), 3.34-3.22 (m, 1H), 3.02-2.97 (m, 1H), 2.67-2.60 (m, 3H), 2.58-2.52 (m, 1H), 2.23-2.11 (m, 1H), 1.97-1.80 (m, 2H), 1.78-1.67 (m, 1H), 1.44-1.39 (m, 3H), 1.09-1.05 (m, 3H).

### Examples 15 and 16. 4-((2S,5R)-5-Ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine and 4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine

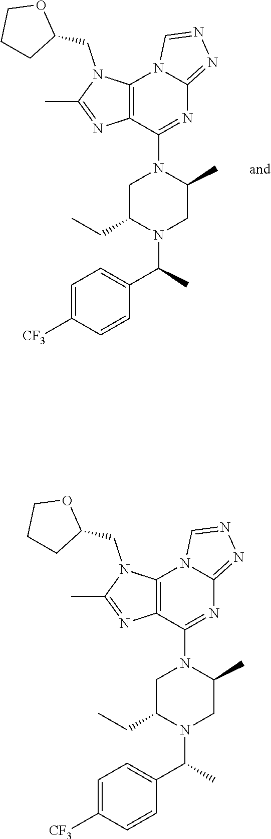 [SMILES: CC[C@@H]1CN(c2nc3nncn3c3c2nc(C)n3C[C@@H]2CCCO2)[C@@H](C)CN1[C@@H](C)c1ccc(C(F)(F)F)cc1 ; CC[C@@H]1CN(c2nc3nncn3c3c2nc(C)n3C[C@@H]2CCCO2)[C@@H](C)CN1[C@H](C)c1ccc(C(F)(F)F)cc1]

### Step 1. 1-(4-(Trifluoromethyl)phenyl)ethyl methanesulfonate

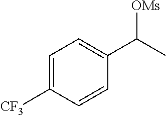 [SMILES: CC(OS(C)(=O)=O)c1ccc(C(F)(F)F)cc1]

To a mixture of 1-(4-(trifluoromethyl)phenyl)ethan-1-ol (0.20 mL, 1.3 mmol) and methanesulfonyl chloride (0.11 mL, 1.43 mmol) in CH₂Cl₂ (5.0 mL) was added triethylamine (0.2 mL, 1.43 mmol), and the reaction mixture was allowed to stir at rt overnight. The reaction mixture was concentrated in vacuo and used directly for next step without further purification.

### Step 2. tert-Butyl (2S,5R)-5-ethyl-2-methyl-4-(1-(4-(trifluoromethyl)phenyl)ethyl)piperazine-1-carboxylate

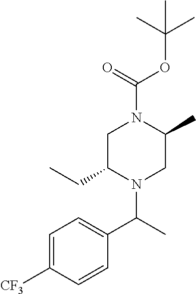 [SMILES: CC[C@@H]1CN(C(=O)OC(C)(C)C)[C@@H](C)CN1C(C)c1ccc(C(F)(F)F)cc1]

To a mixture of tert-butyl (2S,5R)-5-ethyl-2-methylpiperazine-1-carboxylate (Intermediate 41, 228 mg, 1.0 mmol) and 1-(4-(trifluoromethyl)phenyl)ethyl methanesulfonate (Step 1) in MeCN (5.0 mL) was added N,N-diisopropylethylamine (0.35 mL, 2.0 mmol), and the reaction mixture was allowed to stir at 90° C. overnight. The reaction mixture was diluted with EtOAc and water. The layers were separated and the aqueous layer was extracted with EtOAc. The combined organic layers were washed with brine, dried over MgSO₄, concentrated in vacuo, and purified by flash column chromatography (4 g SiO₂, EtOAc/hexanes) to give the desired product as a yellow solid. LC-MS calculated for C₂₁H₃₂F₃N₂O₂(M+H)⁺: m/z=401.2. found 401.2.

### Step 3. (2R,5S)-2-Ethyl-5-methyl-1-(1-(4-(trifluoromethyl)phenyl)ethyl)piperazine hydrochloride

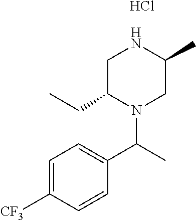 [SMILES: Cl ; CC[C@@H]1CN[C@@H](C)CN1C(C)c1ccc(C(F)(F)F)cc1]

To a mixture of tert-butyl (2S,5R)-5-ethyl-2-methyl-4-(1-(4-(trifluoromethyl)phenyl)ethyl)piperazine-1-carboxylate (Step 2) in CH₂Cl₂ (1.0 mL) was added a 4 molar solution of HCl in 1,4-dioxane (0.5 mL, 2 mmol), and the reaction mixture was allowed to stir at rt for 4 h. The reaction mixture was concentrated in vacuo, and the crude material obtained was used directly without further purification. LC-MS calculated for C₁₆H₂₄F₃N₂ (M+H)⁺: m/z=301.2. found 301.2.

### Step 4. 2-Chloro-6-((2S,5R)-5-ethyl-2-methyl-4-(1-(4-(trifluoromethyl)phenyl)ethyl)piperazin-1-yl)-8-methyl-9-(((S)-tetrahydrofuran-2-yl)methyl)-9H-purine

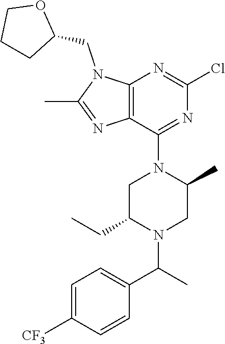 [SMILES: CC[C@@H]1CN(c2nc(Cl)nc3c2nc(C)n3C[C@@H]2CCCO2)[C@@H](C)CN1C(C)c1ccc(C(F)(F)F)cc1]

To a mixture of (S)-2,6-dichloro-8-methyl-9-((tetrahydrofuran-2-yl)methyl)-9H-purine (Intermediate 5, 344 mg, 1.2 mmol) and (2R,5S)-2-ethyl-5-methyl-1-(1-(4-(trifluoromethyl)phenyl)ethyl)piperazine hydrochloride (Step 3) in MeCN (5.0 mL) was added potassium carbonate (276 mg, 2.0 mmol) and the reaction mixture was stirred at 90° C. overnight. After cooling to rt, the reaction mixture was filtered over Celite and the filtrate was concentrated in vacuo. The crude residue was used directly for next step without further purification. LC-MS calculated for C₂₇H₃₅ClF₃N₆O (M+H)⁺: m/z=551.2. found 551.3.

### Step 5. 6-((2S,5R)-5-Ethyl-2-methyl-4-(1-(4-(trifluoromethyl)phenyl)ethyl)piperazin-1-yl)-2-hydrazineyl-8-methyl-9-(((S)-tetrahydrofuran-2-yl)methyl)-9H-purine

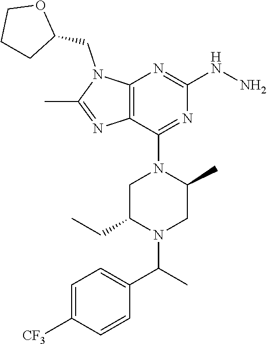 [SMILES: CC[C@@H]1CN(c2nc(NN)nc3c2nc(C)n3C[C@@H]2CCCO2)[C@@H](C)CN1C(C)c1ccc(C(F)(F)F)cc1]

To a mixture of 2-chloro-6-((2S,5R)-5-ethyl-2-methyl-4-(1-(4-(trifluoromethyl)phenyl)ethyl)piperazin-1-yl)-8-methyl-9-(((S)-tetrahydrofuran-2-yl)methyl)-9H-purine (Step 4), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (85 mg, 0.10 mmol), and cesium carbonate (1.63 g, 5.0 mmol) was added a 1 molar solution of hydrazine in THF (5.0 mL, 5.0 mmol) and the mixture was stirred at 90° C. for 30 min. After cooling to rt, the reaction mixture was filtered through a pad of MgSO₄ in a SiliaPrep SPE thiol cartridge (SiliCycle SPE-R51030B-06P). The filtrate was concentrated, and the crude material obtained was used directly without further purification. LC-MS calculated for C₂₇H₃₈F₃N₈O (M+H)⁺: m/z=547.3. found 547.3.

### Step 6. 4-((2S,5R)-5-Ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine and 4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine

A mixture of 6-((2S,5R)-5-ethyl-2-methyl-4-(1-(4-(trifluoromethyl)phenyl)ethyl)piperazin-1-yl)-2-hydrazineyl-8-methyl-9-(((S)-tetrahydrofuran-2-yl)methyl)-9H-purine (Step 5), triethyl orthoformate (1.0 mL, 6.0 mmol), and AcOH (0.034 mL, 0.60 mmol) was stirred at 90° C. for 1 h. After cooling to rt, the reaction mixture was diluted with acetonitrile and water and purified by prep-HPLC (Sunfire C18 column, eluting with a gradient of acetonitrile/water containing 0.1% TFA, at flow rate of 60 mL/min) to afford each diastereomer as its TFA salt.

Example 15: Retention time on LC-MS tᵣ=1.42 min. LC-MS calculated for C₂₈H₃₆F₃N₈O (M+H)⁺: m/z=557.3. found 557.3. ¹H NMR (600 MHz, DMSO-d₆, 70° C.) (mixture of rotamers) δ 9.44-9.41 (m, 1H), 7.77-7.72 (m, 2H), 7.69-7.64 (m, 2H), 6.34-5.77 (m, 2H), 4.76-4.69 (m, 1H), 4.60-4.52 (m, 1H), 4.18-4.09 (m, 1H), 4.07-3.95 (m, 1H), 3.75-3.67 (m, 1H), 3.66-3.42 (m, 2H), 3.11-2.84 (m, 2H), 2.72-2.54 (m, 4H), 2.20-2.10 (m, 1H), 2.00-1.89 (m, 1H), 1.89-1.81 (m, 1H), 1.77-1.67 (m, 1H), 1.59-1.33 (m, 8H), 0.79-0.73 (m, 3H).

Example 16: Retention time on LC-MS tᵣ=1.45 min. LC-MS calculated for C₂₈H₃₆F₃N₈O (M+H)⁺: m/z=557.3. found 557.3. ¹H NMR (600 MHz, DMSO-d₆, 70° C.) (mixture of rotamers) δ 9.45-9.41 (m, 1H), 7.77-7.64 (m, 4H), 6.35-5.94 (m, 1H), 5.19-4.85 (m, 1H), 4.76-4.69 (m, 1H), 4.61-4.52 (m, 1H), 4.18-4.10 (m, 1H), 3.98-3.68 (m, 3H), 3.62-3.54 (m, 1H), 3.39-3.19 (m, 1H), 2.88-2.70 (m, 1H), 2.68-2.56 (m, 3H), 2.41-2.28 (m, 1H), 2.20-2.10 (m, 1H), 1.98-1.80 (m, 2H), 1.80-1.67 (m, 1H), 1.60-1.43 (m, 2H), 1.39-1.31 (m, 6H), 1.04-0.97 (m, 3H).

### Example 17. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

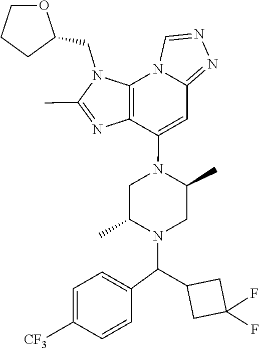

### Step 1. 6-Chloro-4-((2S,5R)-4-((3,3-difluorocyclobutyl)(4-(trifluoromethyl)phenyl)methyl)-2,5-dimethylpiperazin-1-yl)-3-nitropyridin-2-amine

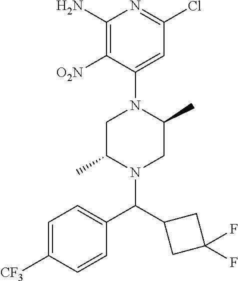 [SMILES: C[C@@H]1CN(c2cc(Cl)nc(N)c2[N+](=O)[O-])[C@@H](C)CN1C(c1ccc(C(F)(F)F)cc1)C1CC(F)(F)C1]

To a mixture of 4,6-dichloro-3-nitropyridin-2-amine (50 mg, 0.24 mmol, ChemScene CS-0094679) and (2R,5S)-1-((3,3-difluorocyclobutyl)(4-(trifluoromethyl)phenyl)methyl)-2,5-dimethylpiperazine hydrochloride (Intermediate 3, 96 mg, 0.24 mmol) in MeCN (2.0 mL) was added N,N-diisopropylethylamine (0.126 mL, 0.721 mmol) and the mixture was stirred at 90° C. overnight. After cooling to rt, the mixture was concentrated in vacuo, and the crude material obtained was used directly without further purification. LC-MS calculated for C₂₃H₂₆ClF₅N₅O₂ (M+H)⁺: m/z=534.2. found 534.3.

### Step 2. 6-Chloro-4-((2S,5R)-4-((3,3-difluorocyclobutyl)(4-(trifluoromethyl)phenyl)methyl)-2,5-dimethylpiperazin-1-yl)pyridine-2,3-diamine

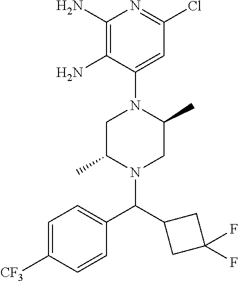 [SMILES: C[C@@H]1CN(c2cc(Cl)nc(N)c2N)[C@@H](C)CN1C(c1ccc(C(F)(F)F)cc1)C1CC(F)(F)C1]

A mixture of 6-chloro-4-((2S,5R)-4-((3,3-difluorocyclobutyl)(4-(trifluoromethyl)phenyl)methyl)-2,5-dimethylpiperazin-1-yl)-3-nitropyridin-2-amine (Step 1) in DMF (2.0 mL) was added hypodiboric acid (64.7 mg, 0.721 mmol), followed by dropwise addition of a solution of 4,4′-dipyridyl (0.38 mg, 2.4 μmol) in DMF (0.5 mL). The mixture was stirred at rt for 10 min, at which point the mixture was diluted with water and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₃H₂₈ClF₅N₅(M+H)⁺: m/z=504.2. found 504.3.

### Step 3. 5-Chloro-7-((2S,5R)-4-((3,3-difluorocyclobutyl)(4-(trifluoromethyl)phenyl)methyl)-2,5-dimethylpiperazin-1-yl)-2-methyl-3H-imidazo[4,5-b]pyridine

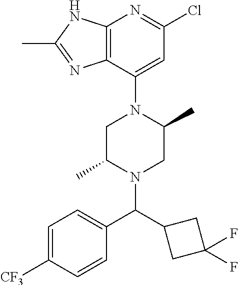 [SMILES: Cc1nc2c(N3C[C@@H](C)N(C(c4ccc(C(F)(F)F)cc4)C4CC(F)(F)C4)C[C@@H]3C)cc(Cl)nc2[nH]1]

A mixture of 6-chloro-4-((2S,5R)-4-((3,3-difluorocyclobutyl)(4-(trifluoromethyl)phenyl)methyl)-2,5-dimethylpiperazin-1-yl)pyridine-2,3-diamine (Step 2) and acetic acid (0.5 mL, 8.73 mmol) in triethyl orthoacetate (0.5 mL, 2.7 mmol) was stirred at 90° C. for 4 h. After cooling to rt, the mixture was diluted with saturated aqueous NaHCO₃ and EtOAc. The organic layer was removed, and the aqueous layer was extracted with EtOAc. The combined organic layers were dried over MgSO₄, filtered, and concentrated under reduced pressure to afford the desired product. The crude material obtained was used directly without further purification. LC-MS calculated for C₂₅H₂₈ClF₅N₅(M+H)⁺: m/z=528.2. found 528.2.

### Step 4. 5-Chloro-7-((2S,5R)-4-((3,3-difluorocyclobutyl)(4-(trifluoromethyl)phenyl)methyl)-2,5-dimethylpiperazin-1-yl)-2-methyl-3-(((S)-tetrahydrofuran-2-yl)methyl)-3H-imidazo[4,5-b]pyridine

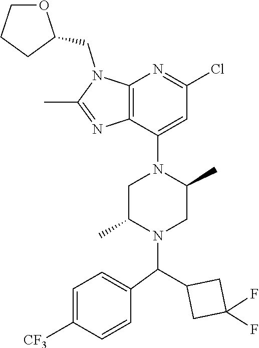 [SMILES: Cc1nc2c(N3C[C@@H](C)N(C(c4ccc(C(F)(F)F)cc4)C4CC(F)(F)C4)C[C@@H]3C)cc(Cl)nc2n1C[C@@H]1CCCO1]

A mixture of 5-chloro-7-((2S,5R)-4-((3,3-difluorocyclobutyl)(4-(trifluoromethyl)phenyl)methyl)-2,5-dimethylpiperazin-1-yl)-2-methyl-3H-imidazo[4,5-b]pyridine (Step 3) in MeCN (2.0 mL) was added cesium carbonate (236 mg, 0.724 mmol) and (S)-(tetrahydrofuran-2-yl)methyl methanesulfonate (Intermediate 50, 87 mg, 0.48 mmol) and the reaction mixture was stirred at 90° C. overnight. After cooling to rt, the mixture was concentrated in vacuo, and the residue was taken up in CH₂Cl₂ and washed with saturated aqueous NaHCO₃. The organic layer was removed, and the aqueous layer was extracted with CH₂Cl₂. The combined organic layers were dried over MgSO₄ and the filtrate was concentrated to afford the desired product as a mixture of diastereomers. The crude material obtained was used directly without further purification. LC-MS calculated for C₃₀H₃₆ClF₅N₅O (M+H)⁺: m/z=612.3. found 612.3.

### Step 5. 7-((2S,5R)-4-((3,3-Difluorocyclobutyl)(4-(trifluoromethyl)phenyl)methyl)-2,5-dimethylpiperazin-1-yl)-5-hydrazineyl-2-methyl-3-(((S)-tetrahydrofuran-2-yl)methyl)-3H-imidazo[4,5-b]pyridine

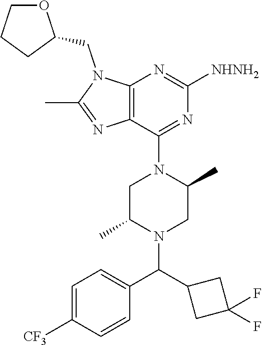 [SMILES: Cc1nc2c(N3C[C@@H](C)N(C(c4ccc(C(F)(F)F)cc4)C4CC(F)(F)C4)C[C@@H]3C)nc(NN)nc2n1C[C@@H]1CCCO1]

To a mixture of 5-chloro-7-((2S,5R)-4-((3,3-difluorocyclobutyl)(4-(trifluoromethyl)phenyl)methyl)-2,5-dimethylpiperazin-1-yl)-2-methyl-3-(((S)-tetrahydrofuran-2-yl)methyl)-3H-imidazo[4,5-b]pyridine (Step 4), cesium carbonate (0.236 g, 0.725 mmol), and methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (21 mg, 0.024 mmol, Aldrich 745979) in 1,4-dioxane (2 mL) was added hydrazine (0.077 mL, 2.4 mmol), and the mixture was purged with nitrogen and stirred at 90° C. for 1 h. After cooling to rt, the reaction mixture was diluted with CH₂Cl₂ and filtered through a pad of MgSO₄ in a SiliaPrep SPE thiol cartridge (500 mg, SiliCycle SPE-R51030B-06P). The filtrate was concentrated, and the crude residue was purified by flash column chromatography (12 g SiO₂, 0-5% MeOH/CH₂Cl₂) to afford the desired product (68 mg, 46% yield over 5 steps) as a mixture of diastereomers in the form of an off-white solid. LC-MS calculated for C₃₀H₃₉F₅N₇O (M+H)⁺: m/z=608.3. found 608.4.

### Step 6. 4-((2S,5R)-4-((3,3-Difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

To a mixture of 7-((2S,5R)-4-((3,3-difluorocyclobutyl)(4-(trifluoromethyl)phenyl)methyl)-2,5-dimethylpiperazin-1-yl)-5-hydrazineyl-2-methyl-3-(((S)-tetrahydrofuran-2-yl)methyl)-3H-imidazo[4,5-b]pyridine (68 mg, 0.11 mmol) in AcOH (1.0 mL, 17 mmol) was added triethyl orthoformate (0.037 mL, 0.224 mmol) and the reaction mixture was stirred at 90° C. for 1 h. After cooling to rt, the reaction mixture was diluted with acetonitrile, water, and several drops of TFA, and the diastereomeric mixture was filtered and purified by prep-HPLC (Sunfire C18 column, eluting with a gradient of acetonitrile/water containing 0.1% TFA, at flow rate of 60 mL/min) to afford the major diastereomer as a single stereoisomer as its TFA salt. LC-MS calculated for C₃₁H₃₇F₅N₇O (M+H)⁺: m/z=618.3. found 618.4.

### Example 18. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

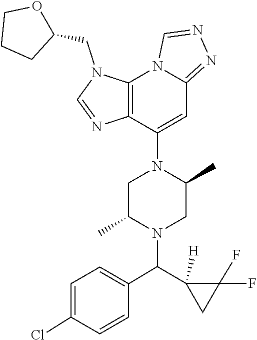 [SMILES: C[C@@H]1CN(c2cc3nncn3c3c2ncn3C[C@@H]2CCCO2)[C@@H](C)CN1C(c1ccc(Cl)cc1)[C@@H]1CC1(F)F]

### Step 1. 7-((2S,5R)-4-((4-Chlorophenyl)((S)-2,2-difluorocyclopropyl)methyl)-2,5-dimethylpiperazin-1-yl)-5-hydrazineyl-3-(((S)-tetrahydrofuran-2-yl)methyl)-3H-imidazo[4,5-b]pyridine

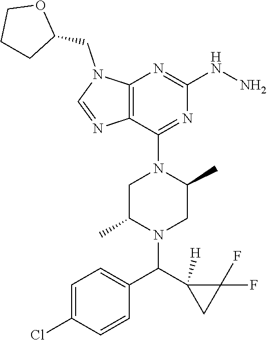 [SMILES: C[C@@H]1CN(c2nc(NN)nc3c2ncn3C[C@@H]2CCCO2)[C@@H](C)CN1C(c1ccc(Cl)cc1)[C@@H]1CC1(F)F]

A mixture 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine (Intermediate 52, 44.9 mg, 0.082 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (7.0 mg, 8.2 μmol, Aldrich 745979), cesium carbonate (80.0 mg, 0.245 mmol), and hydrazine hydrate (7.6 μL, 0.12 mmol, Aldrich 225819) in 1,4-dioxane (0.41 mL) was stirred at 90° C. for 1 h. The mixture was cooled to room temperature, filtered through a pad of MgSO₄ in a SiliaPrep SPE thiol cartridge (SiliCycle SPE-R51030B-06P), concentrated in vacuo, and purified by flash column chromatography (12 g SiO₂, MeOH/DCM) to give the title compound. LC-MS calculated for C₂₇H₃₅ClF₂N₇O (M+H)⁺: m/z=546.3. found 546.4.

### Step 2. 4-((2S,5R)-4-((4-Chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

A mixture of 7-((2S,5R)-4-((4-chlorophenyl)((S)-2,2-difluorocyclopropyl)methyl)-2,5-dimethylpiperazin-1-yl)-5-hydrazineyl-3-(((S)-tetrahydrofuran-2-yl)methyl)-3H-imidazo[4,5-b]pyridine (Step 1), triethyl orthoformate (34 μL, 0.20 mmol), and acetic acid (117 μL, 2.04 mmol) was stirred at 95° C. for 1 h, cooled to room temperature, and directly purified by prep-HPLC (Sunfire C18 column, eluting with a gradient of acetonitrile/water containing 0.1% TFA, at flow rate of 60 mL/min) to afford the title compound as a single stereoisomer as its TFA salt. LC-MS calculated for C₂₈H₃₃ClF₂N₇O (M+H)⁺: m/z=556.2. found 556.3. ¹H NMR (500 MHz, DMSO-d₆) δ 9.55 (s, 1H), 8.25 (s, 1H), 7.48 (d, J=8.2 Hz, 2H), 7.43 (d, J=8.1 Hz, 2H), 6.54 (s, 1H), 5.08-4.91 (m, 2H), 4.84 (dd, J=15.1, 3.0 Hz, 1H), 4.63 (dd, J=15.1, 7.6 Hz, 1H), 4.23 (qd, J=7.2, 2.8 Hz, 1H), 3.74 (dd, J=13.1, 3.6 Hz, 1H), 3.64 (q, J=7.1 Hz, 1H), 3.61-3.55 (m, 2H), 3.38 (d, J=9.8 Hz, 1H), 2.85 (dd, J=12.2, 4.3 Hz, 1H), 2.28 (dd, J=12.2, 3.0 Hz, 1H), 2.21-2.07 (m, 2H), 1.88-1.68 (m, 3H), 1.51 (td, J=9.4, 5.2 Hz, 1H), 1.26 (d, J=6.5 Hz, 3H), 1.11 (d, J=6.4 Hz, 3H), 0.99 (dt, J=11.3, 8.3 Hz, 1H).

### Example 19. 4-((2S,5R)-4-((4-Chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine

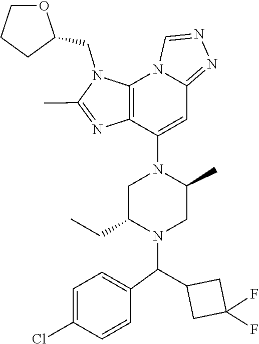

The title compound was prepared according to the procedures outlined in Example 17, with (2R,5S)-1-((4-chlorophenyl)(3,3-difluorocyclobutyl)methyl)-2-ethyl-5-methylpiperazine hydrochloride (Intermediate 43) replacing (2R,5S)-1-((3,3-difluorocyclobutyl)(4-(trifluoromethyl)phenyl)methyl)-2,5-dimethylpiperazine hydrochloride in Step 1. The title compound was isolated as a single stereoisomer as its TFA salt. LC-MS calculated for C₃₁H₃₉ClF₂N₇O (M+H)⁺: m/z=598.3. found 598.4. ¹H NMR (500 MHz, DMSO-d₆) (mixture of rotamers) δ 9.56 (s, 1H), 7.54-7.43 (m, 2H), 7.43-7.31 (m, 2H), δ 6.47 (s, 1H), 5.71-5.08 (m, 1H), 4.84-4.66 (m, 1.6H), 4.66-4.55 (m, 1.4H), 4.38-4.18 (m, 1H), 4.15-4.08 (m, 1H), 3.68-3.64 (m, 1H), 3.58-3.50 (m, 1H), 3.39-3.29 (m, 1H), 3.15-2.99 (m, 1H), 2.87-2.67 (m, 1H), 2.63-2.54 (m, 3H), 2.46-2.31 (m, 4H), 2.30-2.19 (m, 1H), 2.18-2.09 (m, 1H), 2.09-1.96 (m, 1H), 1.96-1.88 (m, 1H), 1.88-1.78 (m, 1H), 1.78-1.68 (m, 1H), 1.60-1.47 (m, 1H), 1.36-1.14 (m, 4H), 0.86-0.72 (m, 3H).

### Examples 20 and 21. 4-((2S,5R)-4-((S)-1-(4-Chlorophenyl)propyl)-2,5-dimethylpiperazin-1-yl)-1-(((S)-tetrahydrofuran-2-yl)methyl)-1H-[1,2,4]triazolo[3,4-b]purine and 4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine

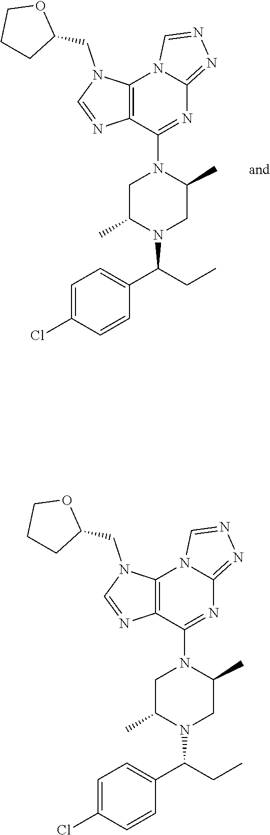 [SMILES: CC[C@@H](c1ccc(Cl)cc1)N1C[C@H](C)N(c2nc3nncn3c3c2ncn3C[C@@H]2CCCO2)C[C@H]1C ; CC[C@H](c1ccc(Cl)cc1)N1C[C@H](C)N(c2nc3nncn3c3c2ncn3C[C@@H]2CCCO2)C[C@H]1C]

The title compounds were prepared according to the procedures outlined in Steps 2-4 for Example 8, with (2R,5S)-1-(1-(4-chlorophenyl)propyl)-2,5-dimethylpiperazine hydrochloride (Intermediate 55) replacing (2R,5S)-1-((4-chlorophenyl)((R)-2,2-difluorocyclopropyl)methyl)-2,5-dimethylpiperazine hydrochloride. The crude reaction mixture was purified by prep-HPLC (Sunfire C18 column, eluting with a gradient of acetonitrile/water containing 0.1% TFA, at flow rate of 60 mL/min) to afford a diastereomeric mixture of the title compounds as TFA salts. The diastereomeric mixture was dissolved in CH₂Cl₂ (2 mL) and 1 M NaOH (5 mL) was added. The layers were separated and the aqueous layer was extracted with CH₂Cl₂ (5×2 mL). The combined organic layers were dried over MgSO₄ and concentrated in vacuo. The residue was purified by normal phase preparative chiral HPLC (CHIRALPAK® IG column, 250×21.2 mm, 5 μm, eluting with a gradient of 30% EtOH in hexanes, at a flow rate of 20 mL/min) to afford the title compounds as separated diastereomers. Each diastereomer was then taken up in MeCN (1 mL) and water (1 mL), several drops of TFA were added, and the mixture was frozen and dried via lyophilization to afford each of the title compounds as its TFA salt.

Example 20: Retention time on CHIRALPAK® IG column tᵣ=17 min, LC-MS calculated for C₂₆H₃₄ClN₈O (M+H)⁺: m/z=509.3. found 509.2.

Example 21: Retention time on CHIRALPAK® IG column tᵣ=24 min, LC-MS calculated for C₂₆H₃₄ClN₈O (M+H)⁺: m/z=509.3. found 509.2.

### Example 22. 4-((2S,5R)-4-((4-Chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine

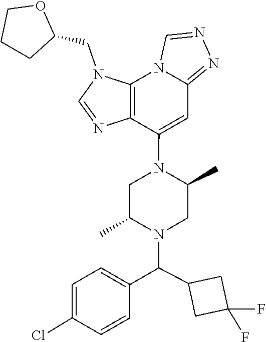

The title compound was prepared according to the procedures outlined in Example 18, with 5-chloro-7-((2S,5R)-4-((4-chlorophenyl)(3,3-difluorocyclobutyl)methyl)-2,5-dimethylpiperazin-1-yl)-3-(((S)-tetrahydrofuran-2-yl)methyl)-3H-imidazo[4,5-b]pyridine (Intermediate 58) replacing 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine in Step 1. The major diastereomer of the title compound was isolated as a single stereoisomer as its TFA salt. LC-MS calculated for C₂₉H₃₅ClF₂N₇O (M+H)⁺: m/z=570.3. found 570.3. ¹H NMR (500 MHz, DMSO-d₆) δ 9.63 (s, 1H), 8.31 (s, 1H), 7.50-7.44 (m, 4H), 6.58 (s, 1H), 5.32-4.72 (m, 3H), 4.63 (dd, J=15.1, 7.7 Hz, 1H), 4.20 (qd, J=7.0, 2.8 Hz, 1H), 3.75-3.37 (m, 4H), 3.25-3.00 (m, 1H), 2.94-2.76 (m, 2H), 2.75-2.55 (m, 2H), 2.48-2.30 (m, 1H), 2.28-2.15 (m, 1H), 2.13-1.96 (m, 2H), 1.85-1.68 (m, 3H), 1.34 (d, J=6.4 Hz, 3H), 1.15-0.87 (m, 3H).

### Example 23. 4-((2S,5R)-4-((4-Chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine

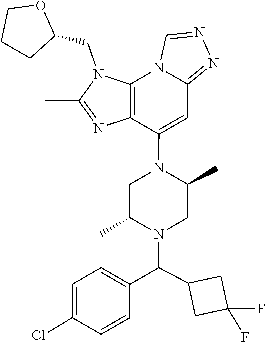

The title compound was prepared according to the procedures outlined in Example 18, with 5-chloro-7-((2S,5R)-4-((4-chlorophenyl)(3,3-difluorocyclobutyl)methyl)-2,5-dimethylpiperazin-1-yl)-2-methyl-3-(((S)-tetrahydrofuran-2-yl)methyl)-3H-imidazo[4,5-b]pyridine (Intermediate 59) replacing 5-chloro-7-((2S,5R)-4-((4-chlorophenyl)((S)-2,2-difluorocyclopropyl)methyl)-2,5-dimethylpiperazin-1-yl)-3-(((S)-tetrahydrofuran-2-yl)methyl)-3H-imidazo[4,5-b]pyridine in Step 1. The major diastereomer of the title compound was isolated as a single stereoisomer as its TFA salt. LC-MS calculated for C₃₀H₃₇ClF₂N₇O (M+H)⁺: m/z=584.3. found 584.2. ¹H NMR (500 MHz, DMSO-d₆) δ 9.59 (s, 1H), 7.56-7.35 (m, 4H), 6.56 (s, 1H), 5.33-4.68 (m, 3H), 4.63 (dd, J=15.9, 9.1 Hz, 1H), 4.12 (q, J=9.0 Hz, 1H), 3.79-3.51 (m, 3H), 3.51-3.36 (m, 1H), 3.29-3.00 (m, 1H), 2.99-2.76 (m, 2H), 2.72-2.57 (m, 2H), 2.51 (s, 3H), 2.48-2.32 (m, 1H), 2.27-2.10 (m, 2H), 2.08-1.97 (m, 1H), 1.97-1.89 (m, 1H), 1.83 (dp, J=11.6, 7.5 Hz, 1H), 1.74 (dq, J=12.0, 8.2 Hz, 1H), 1.33 (d, J=6.2 Hz, 3H), 1.11-0.90 (m, 3H).

### Example 24. 2-(4-((2S,5R)-4-(Bis(4-fluorophenyl)methyl)-2,5-dimethylpiperazin-1-yl)-1H-[1,2,4]triazolo[3,4-b]purin-1-yl)-N,N-dimethylethane-1-amine (i.e., “Compound 3”)

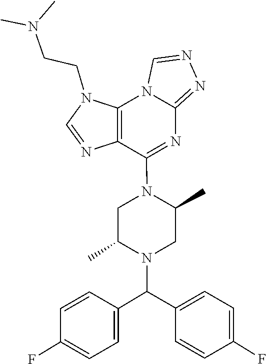 [SMILES: C[C@@H]1CN(c2nc3nncn3c3c2ncn3CCN(C)C)[C@@H](C)CN1C(c1ccc(F)cc1)c1ccc(F)cc1]

### Step 1. 2-(6-((2S,5R)-4-(Bis(4-fluorophenyl)methyl)-2,5-dimethylpiperazin-1-yl)-2-chloro-9H-purin-9-yl)-N,N-dimethylethane-1-amine

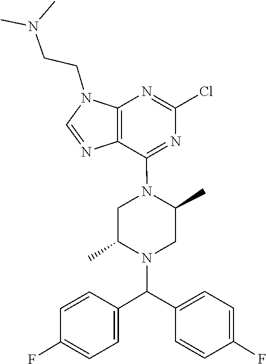 [SMILES: C[C@@H]1CN(c2nc(Cl)nc3c2ncn3CCN(C)C)[C@@H](C)CN1C(c1ccc(F)cc1)c1ccc(F)cc1]

A mixture of 6-((2S,5R)-4-(bis(4-fluorophenyl)methyl)-2,5-dimethylpiperazin-1-yl)-2-chloro-9H-purine (Intermediate 21, 2.33 g, 4.97 mmol) in DMF (24.8 mL) was added cesium carbonate (4.86 g, 14.9 mmol) and 2-bromo-N,N-dimethylethane-1-amine hydrobromide (1.74 g, 7.47 mmol, AstaTech 010701) and the reaction mixture was stirred at 80° C. overnight. After cooling to rt, additional cesium carbonate (1.62 g, 4.97 mmol) and 2-bromo-N,N-dimethylethane-1-amine hydrobromide (1.74 g, 7.47 mmol) was added and the reaction mixture was stirred at 80° C. for 1 h. The reaction mixture was filtered and the filtrate was concentrated. The crude residue was purified by flash column chromatography (40 g SiO₂, eluting with a gradient of 0-10% MeOH in CH₂Cl₂) to afford the desired product (1.45 g, 54% yield) as a yellow waxy solid. LC-MS calculated for C₂₈H₃₃ClF₂N₇(M+H)⁺: m/z=540.2. found 540.2.

### Step 2. 2-(6-((2S,5R)-4-(Bis(4-fluorophenyl)methyl)-2,5-dimethylpiperazin-1-yl)-2-hydrazineyl-9H-purin-9-yl)-N,N-dimethylethane-1-amine

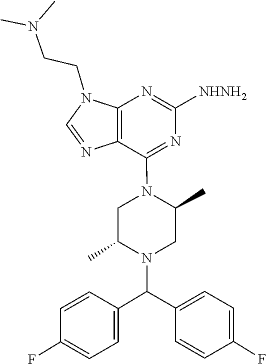 [SMILES: C[C@@H]1CN(c2nc(NN)nc3c2ncn3CCN(C)C)[C@@H](C)CN1C(c1ccc(F)cc1)c1ccc(F)cc1]

A mixture of 2-(6-((2S,5R)-4-(bis(4-fluorophenyl)methyl)-2,5-dimethylpiperazin-1-yl)-2-chloro-9H-purin-9-yl)-N,N-dimethylethane-1-amine (1.45 g, 2.68 mmol), methanesulfonato(2-(di-t-butylphosphino)-3,6-dimethoxy-2′,4′,6′-tri-i-propyl-1,1′-biphenyl)(2′-amino-1,1′-biphenyl-2-yl)palladium(II) (46 mg, 0.054 mmol, Strem 46-0325), and cesium carbonate (1.75 g, 5.37 mmol) in a 1 molar solution of hydrazine in THE (6.71 mL, 6.71 mmol) was purged with nitrogen and stirred at 60° C. for 1 h. After cooling to rt, the reaction mixture was diluted with CH₂Cl₂ and filtered through a pad of MgSO₄ in a SiliaPrep SPE thiol cartridge (SiliCycle SPE-R51030B-06P). The filtrate was concentrated and the crude material obtained was used directly without further purification. LC-MS calculated for C₂₈H₃₆F₂N₉ (M+H)⁺: m/z=536.3. found 536.3.

### Step 3. 2-(4-((2S,5R)-4-(Bis(4-fluorophenyl)methyl)-2,5-dimethylpiperazin-1-yl)-1H-[1,2,4]triazolo[3,4-b]purin-1-yl)-N,N-dimethylethane-1-amine

A mixture of 2-(6-((2S,5R)-4-(bis(4-fluorophenyl)methyl)-2,5-dimethylpiperazin-1-yl)-2-hydrazineyl-9H-purin-9-yl)-N,N-dimethylethane-1-amine (Step 2), triethyl orthoformate (4.5 mL, 27 mmol), and AcOH (154 μL, 2.68 mmol) was stirred at 90° C. overnight. After cooling to rt, the reaction mixture was diluted with acetonitrile and concentrated in vacuo. To the residue was added acetonitrile and water, and the mixture was filtered and purified by prep-HPLC (Sunfire C18 column, eluting with a gradient of acetonitrile/water containing 0.1% TFA, at flow rate of 60 mL/min) to afford the desired product as its TFA salt. Fractions containing the desired product were concentrated, and the material was re-purified by prep-HPLC (Sunfire C18 column, eluting with a gradient of acetonitrile/water containing 0.1% NH₄₀H, at flow rate of 60 mL/min) to afford the desired product. LC-MS calculated for C₂₉H₃₄F₂N₉ (M+H)⁺: m/z=546.3. found 546.3. ¹H NMR (600 MHz, DMSO-d₆) (mixture of rotamers) δ 9.20 (s, 1H), 8.03 (s, 1H), 7.62-7.54 (m, 4H), 7.18-7.11 (m, 4H), 6.19-6.00 (m, 0.4H), 5.89-5.70 (m, 0.6H), 5.16-4.97 (m, 0.6H), 4.65 (s, 1H), 4.62-4.50 (m, 2.4H), 3.82-3.59 (m, 0.6H), 3.55-3.36 (m, 0.4H), 3.20-2.99 (m, 1H), 2.82-2.60 (m, 3H), 2.45-2.34 (m, 1H), 2.15 (s, 6H), 1.42 (d, J=6.6 Hz, 3H), 0.90 (d, J=6.5 Hz, 3H).

### Example A. In Vitro DGKα and DGKζ Inhibition Assays

The DGKα and DGKζ biochemical reactions were performed using His-tagged human recombinant enzymes (Signal Chem, DGKα, #D21-10BH; DGKζ, #D30-10H)) and DLG (Dilauroyl-sn-glycerol) lipid substrate (Signal Chem, #D430-59). ADP-Glo assay was performed using ADP-Glo™ kinase Assay kit (Promega, #V9104). The reactions were carried out in assay buffer containing 40 mM Tris, pH 7.5, 0.1% CHAPS, 0.1% Prionex, 40 mM NaCl, 5 mM MgCl₂, 1 mM CaCl₂, and 1 mM DTT. DGKα reactions contained 0.1 nM DGKα, 50 μM ATP, and 20 μM DLG. And DGKζ reactions contained 0.4 nM DGKζ, 30 μM ATP, and 20 μM DLG.

For compound inhibition studies, 40 nL test compound in DMSO was added to wells of white polystyrene plates in 384-well (Greiner, #784075) or 1536-well format (Greiner, #782075). Compounds were added with top concentration of 2 mM with 11 point, 3-fold dilution series. Enzyme solution (contains 2×DGK enzyme concentration in 1× assay buffer) was added to the plate in 2 μL/well volume, followed by 2 μL/well of substrate solution (contains 2× concentration of ATP and DLG substrate in 1× assay buffer). Plates were then centrifuged for 1 min at 1200 RPM and sealed or lidded. For 4 μL reaction volume, test compounds were therefore diluted 100× to final top concentration of 20 μM. After 90 minute incubation, reactions were quenched by addition of 2 μL/well Promega ADP-Glo Reagent, followed by centrifugation and lidding. After 60 min incubation, 2 μL/well Promega Kinase Detection Reagent was added, plates centrifuged, and incubated for 30 min. Plates were then read using Luminescence method on BMG PHERAstar FSX plate reader. Percent inhibition was calculated and IC50s were determined using 4-parameter fit in Genedata Screener. Labcyte Echo acoustic dispenser was used for compound addition, and Formulatrix Tempest liquid handler was used for all reagent dispenses.

Example DGK inhibitors of the disclosure were tested in one or more of the assays described in Example A, and the resulting data are shown in Table B.

TABLE B

| | DGKα | DGKζ |
|---|---|---|
| Example | IC50 (nM) | IC50 (nM) |
| 1 | + | + |
| 2 | + | + |
| 3 | + | + |
| 4 | + | + |
| 5 | + | + |
| 6 | + | + |
| 7 | + | + |
| 8 | + | + |
| 9 | + | + |
| 10 | + | + |
| 11 | + | + |
| 12 | + | + |
| 13 | + | + |
| 14 | + | + |
| 15 | + | + |
| 16 | + | + |
| 17 | + | + |
| 18 | + | ++ |
| 19 | + | + |
| 20 | + | ++ |
| 21 | + | + |
| 22 | + | + |
| 23 | + | + |
| 24 | + | + |

+ refers to IC₅₀ of ≤20 nM
++ refers to IC₅₀ of >20 nM to ≤200 nM

### Example B. Combinational Effect of DGK Inhibitor with anti-PD-1 Results in Increased Tumor Growth Control In Vivo

### CT26 Model

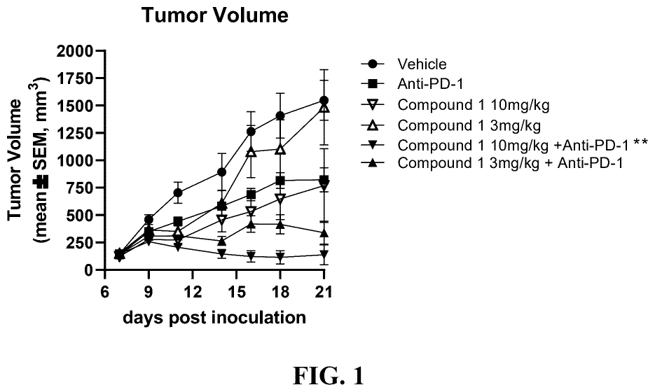

The in vivo effect of combining Compound 1 plus anti-PD-1 (RMP1-14, Bio X Cell) was assessed in the CT26 (CRL-2638, ATCC, Manassas, VA) syngeneic tumor model (see FIG. 1) in 6 to 8 weeks old Balb/C mice (Jackson laboratories). Compound 1 was suspended in 5% N,N-dimethylacetamide (DMAC)+50 mM Citrate buffer in 0.5% methyl cellulose for oral administration and RMP1-14 was suspended in saline for intraperitoneal administration. Briefly, the left flank of the mice was shaved the day prior to subcutaneous inoculation with 1×10⁶ CT26 cells suspended in PBS. On day 7, tumor dimensions were measured by Vernier calipers, and volume estimated by the formula Volume=[L (long dimension)×W² (short dimension)]/2. Mice were randomized into 6 groups of 10 mice of approximate mean volume (˜150 mm³). Tumors were measured 3 times per week for the duration of the study. Starting day 7, mice were dosed with (i) vehicle; (ii) 250 μg/mouse of RMP1-14; (iii) 10 mg/kg of Compound 1; (iv) 3 mg/kg of Compound 1; (v) 10 mg/kg of Compound 1 and 250 μg/mouse of RMP1-14; or (vi) 3 mg/kg of Compound 1 and 250 μg/mouse of RMP1-14. Compound 1 was administered orally once daily (QD) for 14 days, while RMP1-14 was dosed every 5 days (for a total of 3 doses). As shown in FIG. 1, 10 mg/kg of Compound 1 alone had a therapeutic effect in delaying tumor growth when compared to the control vehicle group. The combination of either 10 mg/kg or 3 mg/kg Compound 1 with 250 μg RMP1-14 results in a synergistic effect in tumor growth delay when compared to their respective controls determined by Bliss Independence analysis.

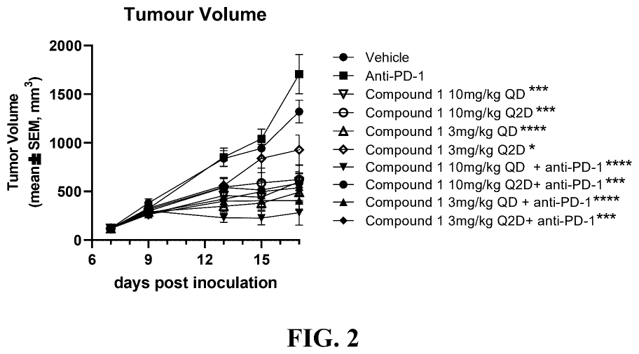

In a separate experiment, mice were randomized on day 7 into 10 groups of 10 mice of approximate mean volume (˜120 mm³). Tumors were measured 3 times a week for the duration of the efficacy study (day 22). Starting day 7, mice were dosed with (i) vehicle; (ii) 250 μg/mouse of RMP1-14; (iii) 10 mg/kg of Compound 1 QD; (iv) 10 mg/kg of Compound 1 Q2D; (v) 3 mg/kg of Compound 1 QD; (vi) 3 mg/kg of Compound 1 QD; (vii) 10 mg/kg of Compound 1 QD and 250 μg/mouse of RMP1-14; (viii) 10 mg/kg of Compound 1 Q2D and 250 μg/mouse of RMP1-14; (ix) 3 mg/kg of Compound 1 QD and 250 μg/mouse of RMP1-14; or (x) 3 mg/kg of Compound 1 Q2D and 250 μg/mouse of RMP1-14. Compound 1 was administered orally either daily (QD) or every other day (Q2D) for 10 days, while RMP1-14 was dosed every 5 days (for a total of 2 doses). As shown in FIG. 2, both 10 and 3 mg/kg of Compound 1 alone had a therapeutic effect in delaying tumor growth when dosed QD compared to the control vehicle group. The combination of either 10 mg/kg or 3 mg/kg Compound 1 with 250 μg RMP1-14 resulted in a synergistic effect in tumor growth delay when dosed either QD or Q2D when compared to their respective controls determined by Bliss Independence analysis (see FIG. 2).

### MC38 Model

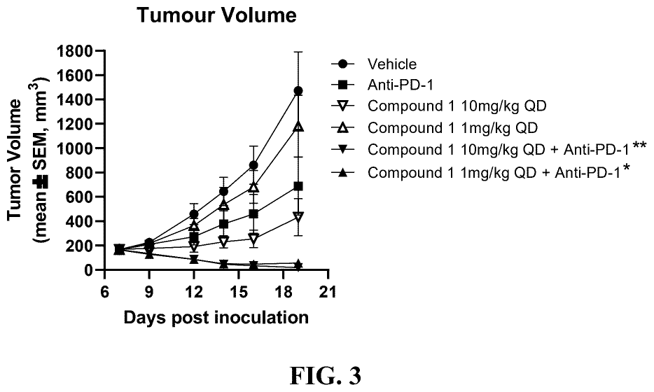

The in vivo effect of combining Compound 1 plus anti-PD-1 (RMP1-14, Bio X Cell) was assessed in the MC38 (CRL-1642, ATCC, Manassas, VA) syngeneic tumor model (see FIG. 3) in 6 to 8 weeks old C57Bl/6 mice (Jackson laboratories). Compound 1 was suspended in 5% N,N-dimethylacetamide (DMAC)+50 mM Citrate buffer in 0.5% methyl cellulose for oral administration and RMP1-14 was suspended in saline for intraperitoneal administration. Briefly, the left flank of the mice was shaved the day prior to subcutaneous inoculation with 2×10⁶ MC38 cells suspended in PBS. On day 7, tumor dimensions were measured by Vernier calipers, and volume estimated by the formula Volume=[L (long dimension)×W² (short dimension)]/2. Mice were randomized into 6 groups of 10 mice of approximate mean volume (˜160 mm³). Tumors were measured 3 times per week for the duration of the study. Starting day 7, mice were dosed with (i) vehicle; (ii) 250 μg/mouse of RMP1-14; (iii) 10 mg/kg of Compound 1; (iv) 1 mg/kg of Compound 1; (v) 10 mg/kg of Compound 1 and 250 μg/mouse of RMP1-14; or (vi) 1 mg/kg of Compound 1 and 250 μg/mouse of RMP1-14. Compound 1 was administered orally once daily (QD) for 12 days, while RMP1-14 was dosed every 5 days (for a total of 3 doses). As shown in FIG. 3, the combination of either 10 mg/kg or 1 mg/kg Compound 1 with 250 μg RMP1-14 results in an additive effect in tumor growth delay when compared to their respective controls determined by Bliss Independence analysis.

### Example C. Human PD-1/Human PD-L1 Dual Knock-In Model

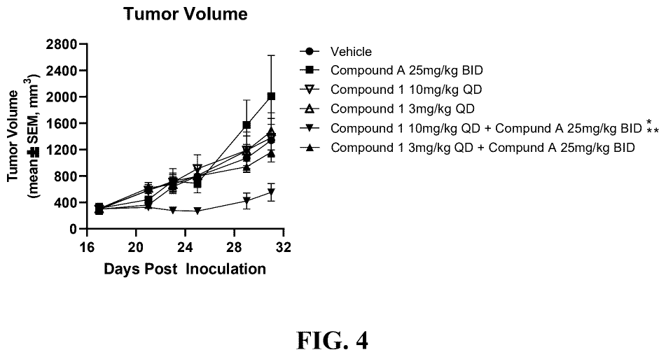

The in vivo effect of combining Compound 1 plus (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid (i.e., Compound A) was assessed in 8 to 10 weeks old human PD-1/human PD-L1 dual Knock-In mice (GemPharmatech, Shanghai, China) using CT26 syngeneic tumors overexpressing human PD-L1 (Clone299, Synthego, California). Both Compound 1 and Compound A were suspended in 5% N,N-dimethylacetamide (DMAC)+50 mM Citrate buffer in 0.5% methyl cellulose for oral administration. Briefly, the left flank of the mice was shaved the day prior to subcutaneous inoculation with 1×10⁶ CT26 Clone299 cells suspended in PBS. On day 17, tumor dimensions were measured by Vernier calipers, and volume estimated by the formula Volume=[L (long dimension)×W² (short dimension)]/2. Mice were randomized into 6 groups of 7 mice of approximate mean volume (˜300 mm³). Tumors were measured 3 times per week for the duration of the study. Starting day 17, mice were dosed with (i) vehicle; (ii) 25 mg/kg of Compound A; (iii) 10 mg/kg of Compound 1; (iv) 3 mg/kg of Compound 1; (v) 10 mg/kg of Compound 1 and 25 mg/kg of Compound A; or (vi) 3 mg/kg of Compound 1 and 25 mg/kg of Compound A. Compound 1 was administered orally once daily (QD) for 15 days, while Compound A was dosed twice a days (BID) for 15 days. As shown in FIG. 4, the combination of 10 mg/kg Compound 1 with 25 mg/kg Compound A BID results in a synergistic effect in tumor growth delay when compared to their respective controls determined by Bliss Independence analysis.

### Example D. Combinational Effect Compound 2 with Anti-PD-1 Results in Increased T Cell Cytokine Production In Vitro

To assess the ability of 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine (i.e., Compound 2) to further enhance the T cell effector function induced by checkpoint blockade, the combination effect of Compound 2 and anti-PD-1 reagent (retifanlimab and pembrolizumab) was evaluated in an allogenic mixed lymphocyte reaction assay.

In brief, Human PBMCs were isolated from leukopaks (BioIVT) obtained from healthy donors by the Ficoll-Paque (Cytiva 17144002) cell separation method. Monocyte isolation was performed using the Dynabeads Untouched human monocyte kit (Life Technologies 11350D), and monocytes were cultured in 10% FBS RPMI 1640 treated with 100 ng/mL granulocyte-macrophage colony-stimulating factor (R&D Systems 215-GM/CF) and 50 ng/mL IL-4 (R&D Systems 204-IL/CF) for at least 6 days at 37° C. with 5% CO₂ to differentiate into dendritic cells. Pan T cells were isolated using the Dynabeads Untouched human T-cell kit (Life Technologies 11344D) and plated at 1×10⁵ cells/well in 100 μL of 10% FBS RPMI 1640 in 96-well, round-bottom plates (Costar 3879). Then, 100 μL of 1×10⁵ cells/mL of allogeneic monocyte-derived dendritic cells were added to the T cells at a 1:10 ratio in the presence of Compound 2 alone or in combination with the checkpoint inhibitors. The co-culture plate was placed in the incubator for 4 days and 16 μL of supernatant from each well was transferred to a 384-well white plate (Greiner 784075). To each well, 4 μL of a 1:1 mixture of diluted donor fluorophore solution and diluted acceptor fluorophore solution from the HTRF human IFNγ detection kit (Revvity 62HIFNGPEH) was added to a final volume of 20 μL per well. After 20 hours of incubation at room temperature, the HTRF signal was measured by the PHERAstar FSX microplate reader (BMG Labtech). Data analysis was performed in GraphPad Prism.

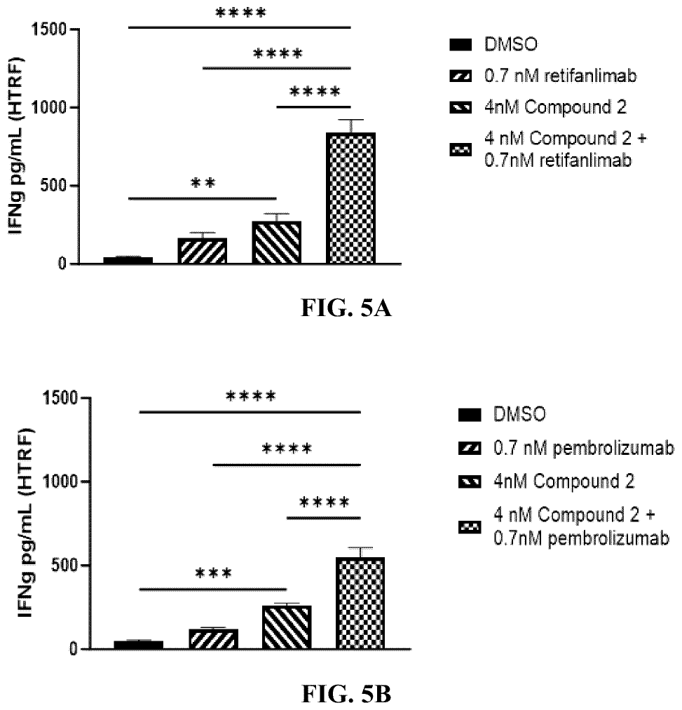

Results indicated that the combination treatment of Compound 2 plus retifanlimab (FIG. 5A), and Compound 2 plus pembrolizumab (FIG. 5B) resulted in statistically significant higher production of IFNγ than anti-PD-1 mAb or Compound 2 single agent treatment. Therefore, this data indicates Compound 2 combines well with anti-PD-1 mAb activity in vitro to further enhance T cell activation.

### Example E. Combinational Effect of Compound 2 with Anti-PD-1 Results in Increased Tumor Growth Control In Vivo

### CT26 Model

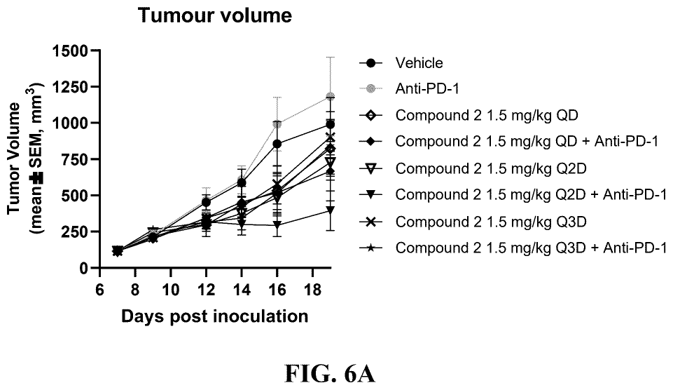

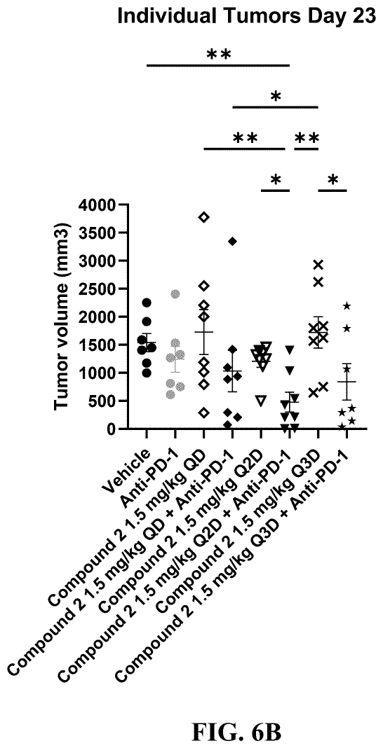

The in vivo effect of combining Compound 2 plus anti-PD-1 (RMP1-14, Bio X Cell) was assessed in the CT26 (CRL-2638, ATCC, Manassas, VA) syngeneic tumor model (see FIG. 6A-6B) in 6 to 8 weeks old Balb/C mice (Jackson laboratories). Compound 2 was suspended in 5% N,N-dimethylacetamide (DMAC)+50 mM Citrate buffer in 0.5% methyl cellulose for oral administration and RMP1-14 was suspended in saline for intraperitoneal administration. Briefly, the left flank of the mice was shaved the day prior to subcutaneous inoculation with 1×10⁶ CT26 cells suspended in PBS. On day 7, tumor dimensions were measured by Vernier calipers, and volume estimated by the formula Volume=[L (long dimension)×W² (short dimension)]/2. Mice were randomized into 6 groups of 10 mice of approximate mean volume (˜130 mm³). Tumors were measured 3 times per week for the duration of the study. Starting day 7, mice were dosed with (i) vehicle; (ii) 250 μg/mouse of RMP1-14 (Anti-PD-1); (iii) 1.5 mg/kg of Compound 2 QD; (iv) 1.5 mg/kg of Compound 2 QD and 250 μg/mouse of RMP1-14; (v) 1.5 mg/kg of Compound 2 Q2D; (vi) 1.5 mg/kg of Compound 2 Q2D and 250 μg/mouse of RMP1-14; (vii) 1.5 mg/kg of Compound 2 Q3D; or (viii) 1.5 mg/kg of Compound 2 Q3D and 250 μg/mouse of RMP1-14. None of the single treatment arm groups dosed with 1.5 mg/kg Compound 2 provided statistically significant TGI compared with the vehicle control group (FIG. 6B). However, when combined with anti-PD-1 mAb, Compound 2 induced significant TGI at 1.5 mg/kg Q2D (p=0.0036). In addition, all the combination groups were statistically significant compared with the respective single-arm control group.

### MC38 Model

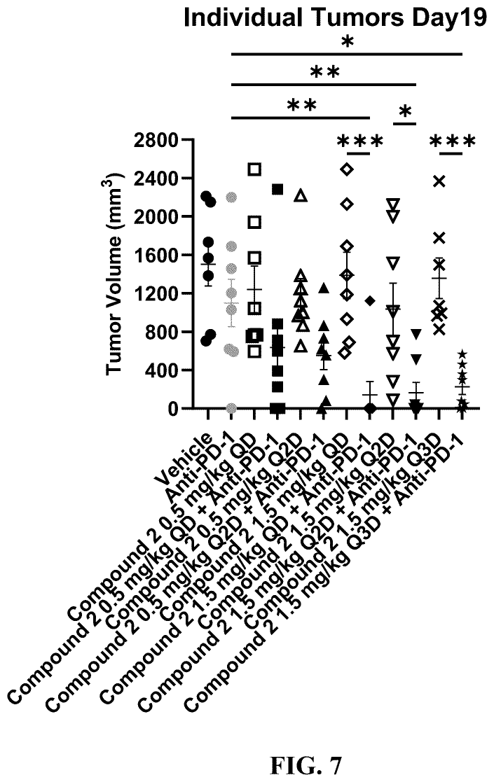

The in vivo effect of combining Compound 2 plus anti-PD-1 (RMP1-14, Bio X Cell) was assessed in the MC38 (CRL-1642, ATCC, Manassas, VA) syngeneic tumor model in 6 to 8 weeks old C57Bl/6 mice (Jackson laboratories). Compound 2 was suspended in 5% N,N-dimethylacetamide (DMAC)+50 mM Citrate buffer in 0.5% methyl cellulose for oral administration and RMP1-14 was suspended in saline for intraperitoneal administration. Briefly, the left flank of the mice was shaved the day prior to subcutaneous inoculation with 2×10⁶ MC38 cells suspended in PBS. On day 7, tumor dimensions were measured by Vernier calipers, and volume estimated by the formula Volume=[L (long dimension)×W² (short dimension)]/2. Mice were randomized into 6 groups of 10 mice of approximate mean volume (˜130 mm³). Tumors were measured 3 times per week for the duration of the study. Starting day 7, mice were dosed with (i) vehicle; (ii) 250 μg/mouse of RMP1-14 (Anti-PD-1); (iii) 0.5 mg/kg of Compound 2 QD; (iv) 0.5 mg/kg of Compound 2 QD and 250 μg/mouse of RMP1-14; (v) 0.5 mg/kg of Compound 2 Q2D; (vi) 0.5 mg/kg of Compound 2 QD and 250 μg/mouse of RMP1-14; (vii) 1.5 mg/kg of Compound 2 QD; (viii) 1.5 mg/kg of Compound 2 QD and 250 μg/mouse of RMP1-14 (ix) 1.5 mg/kg of Compound 2 Q2D; (x) 1.5 mg/kg of Compound 2 Q2D and 250 μg/mouse of RMP1-14; (xi) 1.5 mg/kg of Compound 2 Q3D; or (xii) 1.5 mg/kg of Compound 2 Q3D and 250 μg/mouse of RMP1-14. None of the single treatment arm groups dosed with Compound 2 0.5 or 1.5 mg/kg provided statistically significant TGI compared with both vehicle and anti-PD-1 mAb control groups (see FIG. 7). However, when combined with anti-PD-1 mAb, Compound 2 induced significant TGI at both 0.5 and 1.5 mg/kg, independent of the dose schedule, when compared with the vehicle control group. More importantly, Compound 2 1.5 mg/kg in combination with anti-PD-1 mAb induced significant TGI compared with the anti-PD-1 mAb control group. In addition, all the combination groups were statistically significant compared with the respective single-arm Compound 2 control group.

### Example F. Human PD-1/Human PD-L1 Dual Knock-In Model

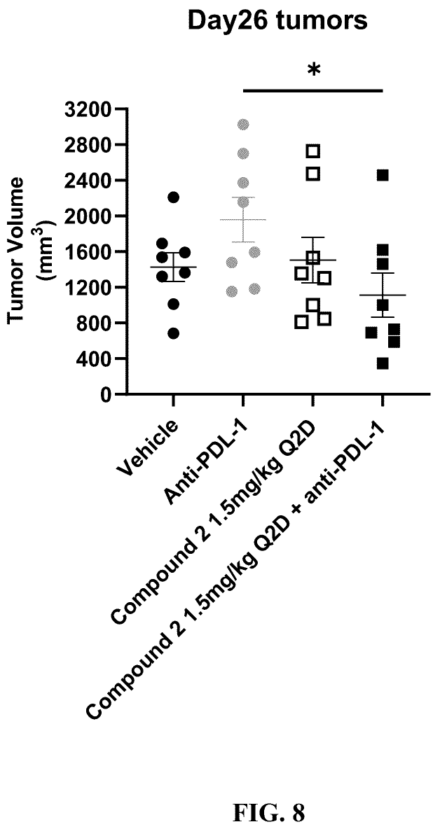

The in vivo effect of Compound 2 plus Anti-PD-L1 (atezolizumab) was assessed in 8 to 10 weeks old human PD-1/human PD-L1 dual Knock-In mice (GemPharmatech, Shanghai, China) using CT26 syngeneic tumors overexpressing human PD-L1 (Clone299, Synthego, California). Compound 2 was suspended in 5% N,N-dimethylacetamide (DMAC)+50 mM Citrate buffer in 0.5% methyl cellulose for oral administration. Briefly, the left flank of the mice was shaved the day prior to subcutaneous inoculation with 1×10⁶ CT26 Clone299 cells suspended in PBS. On day 12, tumor dimensions were measured by Vernier calipers, and volume estimated by the formula Volume=[L (long dimension)×W² (short dimension)]/2. Mice were randomized into 6 groups of 7 mice of approximate mean volume (˜110 mm³). Tumors were measured 3 times per week for the duration of the study. Starting day 12, mice were dosed with (i) vehicle; (ii) Anti-PD-L1; (iii) 1.5 mg/kg of Compound 2 Q2D; or (iv) 1.5 mg/kg of Compound 2 Q2D and Anti-PD-L1. When combined with Anti-PD-L1 mAb, Compound 2 induced significant TGI at 1.5 mg/kg Q2D, when compared with the Anti-PD-L1 mAb control group (FIG. 8).

### Example G. Combinational Effect of Compound 3 with anti-PD-1 Results in Increased Tumor Growth Control In Vivo

### MC38 Model

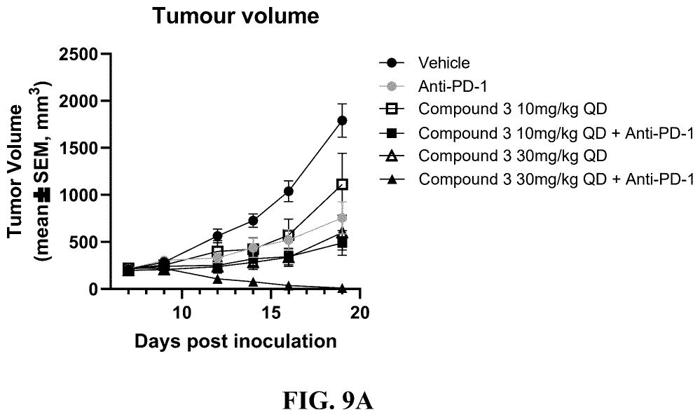

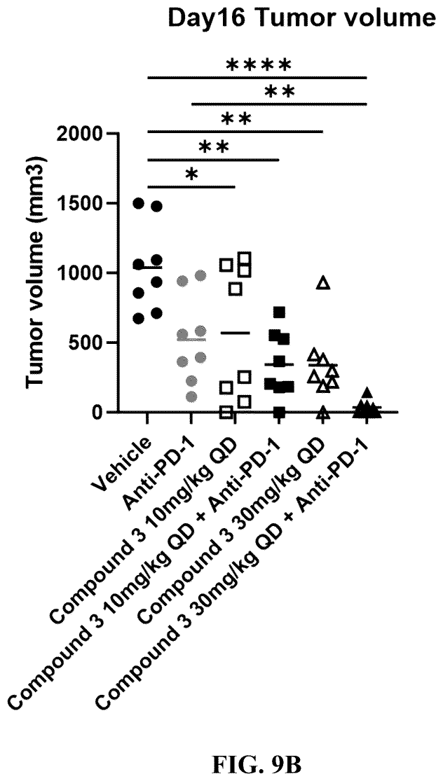

The in vivo effect of combining 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine (i.e., Compound 3) plus anti-PD-1 (RMP1-14, Bio X Cell) was assessed in the MC38 (CRL-1642, ATCC, Manassas, VA) syngeneic tumor model in 6 to 8 weeks old C57Bl/6 mice (Jackson laboratories). Compound 3 was suspended in 5% N,N-dimethylacetamide (DMAC)+50 mM Citrate buffer in 0.5% methyl cellulose for oral administration and RMP1-14 was suspended in saline for intraperitoneal administration. Briefly, the left flank of the mice was shaved the day prior to subcutaneous inoculation with 2×10⁶ MC38 cells suspended in PBS. On day 7, tumor dimensions were measured by Vernier calipers, and volume estimated by the formula Volume=[L (long dimension)×W² (short dimension)]/2. Mice were randomized into 6 groups of 10 mice of approximate mean volume (˜200 mm³). Tumors were measured 3 times per week for the duration of the study. Starting day 7, mice were dosed with (i) vehicle; (ii) 250 μg/mouse of RMP1-14 (Anti-PD-1); (iii) 10 mg/kg of Compound 3 QD; (iv) 10 mg/kg of Compound 3 QD and 250 μg/mouse of RMP1-14; (v) 30 mg/kg of Compound 3 QD; or (vi) 30 mg/kg of Compound 3 QD and 250 μg/mouse of RMP1-14. All treated groups with Compound 3 provided statistically significant TGI compared with vehicle control group (see FIGS. 9A-9B). However, when combined with anti-PD-1 mAb, Compound 3 induced significant TGI at 30 mg/kg when compared with the anti-PD-1 control group.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating cancer in a subject, comprising administering to the subject:
(i) a DGK inhibitor; and
(ii) an inhibitor of PD-1/PD-L1;
wherein the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof.

2. The method of claim 1, wherein DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof.

3. The method of claim 1, wherein the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof.

4. The method of claim 1, wherein the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

5. The method of claim 1, wherein the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof.

6. The method of claim 1, wherein the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof.

7. The method of claim 1, wherein the DGK inhibitor is selected from:
4-((2S,5R)-4-((3,3-difluorocyclobutyl)(4-(trifluoromethyl)phenyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((R)-2,2-difluorocyclopropyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)((S)-2,2-difluorocyclopropyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(1-(4-chlorophenyl)-3-methylbutyl)-2,5-dimethylpiperazin-1-yl)-1-(((S)-tetrahydrofuran-2-yl)methyl)-1H-[1,2,4]triazolo[3,4-b]purine;
4-((2S,5R)-4-(1-(4-chloro-3-fluorophenyl)-3-methylbutyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-5-ethyl-2-methylpiperazin-1-yl)-1-(((S)-tetrahydrofuran-2-yl)methyl)-1H-[1,2,4]triazolo[3,4-b]purine;
4-((2S,5R)-4-(2-fluoro-4-(trifluoromethyl)benzyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((S)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-5-ethyl-2-methyl-4-((R)-1-(4-(trifluoromethyl)phenyl)ethyl)piperazin-1-yl)-2-methyl-1-(((S)-tetrahydrofuran-2-yl)methyl)-1H-[1,2,4]triazolo[3,4-b]purine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((4-chlorophenyl)((S)-2,2-difluorocyclopropyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine;
4-((2S,5R)-4-((4-chlorophenyl)(3,3-difluorocyclobutyl)methyl)-5-ethyl-2-methylpiperazin-1-yl)-2-methyl-1-(((S)-tetrahydrofuran-2-yl)methyl)-1H-imidazo[4,5-e][1,2,4]triazolo[4,3-a]pyridine;
4-((2S,5R)-4-((S)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((R)-1-(4-chlorophenyl)propyl)-2,5-dimethylpiperazin-1-yl)-1-(((S)-tetrahydrofuran-2-yl)methyl)-1H-[1,2,4]triazolo[3,4-b]purine;
4-((2S,5R)-4-((4-chlorophenyl)(3,3-difluorocyclobutyl)methyl)-2,5-dimethylpiperazin-1-yl)-1-(((S)-tetrahydrofuran-2-yl)methyl)-1H-imidazo[4,5-e][1,2,4]triazolo[4,3-a]pyridine; and
4-((2S,5R)-4-((4-chlorophenyl)(3,3-difluorocyclobutyl)methyl)-2,5-dimethylpiperazin-1-yl)-2-methyl-1-(((S)-tetrahydrofuran-2-yl)methyl)-1H-imidazo[4,5-e][1,2,4]triazolo[4,3-a]pyridine;
or a pharmaceutically acceptable salt thereof.

8. The method of at claim 1, wherein the inhibitor of PD-1/PD-L1 is selected from:
(S)-1-((7-chloro-2-(2′-chloro-3′-(5-(((2-hydroxyethyl)amino)methyl)picolinamido)-2-methyl-[1,1′-biphenyl]-3-yl)benzo[d]oxazol-5-yl)methyl)piperidine-2-carboxylic acid;
(S)-1-((7-chloro-2-(3′-(7-chloro-5-(((S)-3-hydroxypyrrolidin-1-yl)methyl)benzo[d]oxazol-2-yl)-2,2′-dimethylbiphenyl-3-yl)benzo[d]oxazol-5-yl)methyl)pyrrolidine-3-carboxylic acid;
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid;
(R)-1-((7-cyano-2-(2,2′-dimethyl-3′-(4,5,6,7-tetrahydrothiazolo[5,4-c]pyridin-2-yl)biphenyl-3-yl)benzo[d]oxazol-5-yl)methyl)pyrrolidine-3-carboxylic acid;
(R)-1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)pyrrolidine-3-carboxylic acid;
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid;
(S)-1-((2-(2′-chloro-3′-(1,5-dimethyl-4,5,6,7-tetrahydro-1H-imidazo[4,5-c]pyridine-2-carboxamido)-2-methylbiphenyl-3-yl)-7-cyanobenzo[d]oxazol-5-yl)methyl)pyrrolidine-3-carboxylic acid;
(R)-1-((2-(2′-chloro-3′-(6-isopropyl-4,5,6,7-tetrahydro-2H-pyrazolo[3,4-c]pyridin-2-yl)-2-methylbiphenyl-3-yl)-7-cyanobenzo[d]oxazol-5-yl)methyl)pyrrolidine-3-carboxylic acid;
(S)—N-(2-chloro-3′-(5-(2-hydroxypropyl)-1-methyl-4,5,6,7-tetrahydro-1H-imidazo[4,5-c]pyridine-2-carboxamido)-2′-methylbiphenyl-3-yl)-5-isopropyl-1-methyl-4,5,6,7-tetrahydro-1H-imidazo[4,5-c]pyridine-2-carboxamide;
cis-4-((2-((2,2′-dichloro-3′-(1-methyl-5-(tetrahydro-2H-pyran-4-yl)-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid;
trans-4-(2-(2-((2,2′-dichloro-3′-(5-(2-hydroxyethyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
trans-4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid;
(R)-1-((7-cyano-2-(3′-(7-((3-hydroxypyrrolidin-1-yl)methyl)-2-methylpyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(7-(((S)-1-hydroxypropan-2-ylamino)methyl)-2-methylpyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-N,N-dimethylpiperidine-4-carboxamide;
(R)-1-((7-cyano-2-(3′-(2-cyclopropyl-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(3-(((R)-3-hydroxypyrrolidin-1-yl)methyl)-6-methyl-1,7-naphthyridin-8-ylamino)-2,2′-dimethylbiphenyl-3-yl)benzo[d]oxazol-5-yl)methyl)pyrrolidine-3-carboxylic acid;
4-(2-(2-((2,2′-dichloro-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((3′-(5-((1H-pyrazol-3-yl)methyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid);
4-(2-(2-((2-chloro-2′-methyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2,2′-dimethyl-3′-(1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((3′-(5-(trans-4-carboxy-4-methylcyclohexyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((2-chloro-3′-((2-(difluoromethyl)-7-((3-hydroxy-3-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(pyrrolidin-1-ylmethyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(R)-4-(2-(2-((2-chloro-3′-((2-(difluoromethyl)-7-((2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((((1R,2S)-2-hydroxycyclopentyl)amino)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2S,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4R)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-(((2R,4S)-4-hydroxy-2-methylpyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxyazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4-(2-(2-((2-chloro-3′-((2-(difluoromethyl)-7-((3-hydroxy-3-methylazetidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
(S)-4-(2-(2-((3′-((2-(difluoromethyl)-7-((3-hydroxypyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid; and
(S)-4-(2-(2-((3′-((2-(difluoromethyl)-7-((3-hydroxy-3-methylpyrrolidin-1-yl)methyl)pyrido[3,2-d]pyrimidin-4-yl)amino)-2,2′-dimethyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
or a pharmaceutically acceptable salt thereof.

9. The method of claim 1, wherein the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

10. The method of claim 1, wherein the inhibitor of PD-1/PD-L1 is 4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid), or a pharmaceutically acceptable salt thereof.

11. The method of claim 1, wherein the inhibitor of PD-1/PD-L1 is (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid, or a pharmaceutically acceptable salt thereof.

12. The method of claim 1, wherein the inhibitor of PD-1/PD-L1 is selected from retifanlimab, nivolumab, and pembrolizumab.

13. The method of claim 1, wherein the inhibitor of PD-1/PD-L1 is retifanlimab.

14. The method of claim 1, wherein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof; and
(ii) the inhibitor of PD-1/PD-L1 is selected from:
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
or a pharmaceutically acceptable salt thereof.

15. The method of claim 1, wherein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

16. The method of claim 1, wherein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

17. The method of claim 1, wherein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is (R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid, or a pharmaceutically acceptable salt thereof.

18. The method of claim 1, wherein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(Bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof; and
(ii) the inhibitor of PD-1/PD-L1 is selected from retifanlimab, nivolumab, pembrolizumab, atezolizumab, and RMP1-14.

19. The method of claim 1, wherein:
(i) the DGK inhibitor is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine;
or a pharmaceutically acceptable salt thereof; and
(ii) the inhibitor of PD-1/PD-L1 is selected from retifanlimab, nivolumab, and pembrolizumab.

20. The method of claim 1, wherein:
(i) the DGK inhibitor is 4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine, or a pharmaceutically acceptable salt thereof; and
(ii) the inhibitor of PD-1/PD-L1 is retifanlimab.

21. The method of claim 1, wherein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof; and
(ii) the inhibitor of PD-1/PD-L1 is retifanlimab.

22. The method of claim 1, wherein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
(ii) the inhibitor of PD-1/PD-L1 is pembrolizumab.

23. The method of claim 1, wherein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
(ii) the inhibitor of PD-1/PD-L1 is RMP1-14.

24. The method of claim 1, wherein:
(i) the DGK inhibitor is 4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine; and
(ii) the inhibitor of PD-1/PD-L1 is atezolizumab.

25. The method of claim 1, wherein:
(i) the DGK inhibitor is 4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine, or a pharmaceutically acceptable salt thereof, and
(ii) the inhibitor of PD-1/PD-L1 is retifanlimab.

26. The method of claim 1, wherein:
(i) the DGK inhibitor is 2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine, or a pharmaceutically acceptable salt thereof; and
(ii) the inhibitor of PD-1/PD-L1 is RMP1-14.

27. The method of claim 1, wherein the cancer is colon cancer, non-small cell lung cancer, bladder urothelial carcinoma, esophageal carcinoma, stomach adenocarcinoma, mesothelioma, liver hepatocellular carcinoma, diffuse large B cell lymphoma, kidney renal clear cell carcinoma, head and neck squamous cell carcinoma, cholangiocarcinoma, cervical squamous cell carcinoma, endocervical adenocarcinoma, and melanoma.

28. The method of claim 27, wherein the melanoma is metastatic melanoma.

29. The method of claim 27, wherein the cancer is colon cancer.

30. The method of claim 1, wherein the cancer is a tumor.

31. The method of claim 30, wherein the tumor comprises high microsatellite instability (MSI-H), mismatch repair deficient (MMRd), high tumor mutational burden (TMB-H), or mismatch repair deficient (MMRd) and high tumor mutational burden (TMB-H).

32. The method of claim 1, wherein the DGK inhibitor and the inhibitor of PD-1/PD-L1 are administered simultaneously.

33. The method of claim 1, wherein the DGK inhibitor and the inhibitor of PD-1/PD-L1 are administered sequentially.

34. A method of treating cancer in a subject, comprising administering to the subject a compound of Formula I: 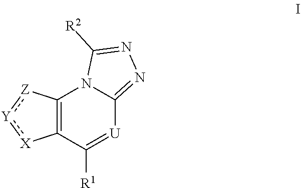
or a pharmaceutically acceptable salt thereof, wherein:
each [symbol] is a single or double bond, wherein at least one [symbol] is a double bond;
U is CH or N;
X is CR⁴, N, NR⁴, S, or O;
Y is CR⁵ or N;
Z is CR⁶, N⁶, or S;
R¹ is Cy¹ or L-Cy¹;
L is NR^{c7}, O, C₁₋₃ alkyl, C₂₋₃ alkenyl, or C₂₋₃ alkynyl;
Cy¹ is a C₃₋₁₀ cycloalkyl, 5-15 membered heteroaryl, or 4-15 membered heterocycloalkyl, wherein the C₃₋₁₀ cycloalkyl, 5-15 membered heteroaryl or 4-15 membered heterocycloalkyl are each optionally substituted with 1, 2, 3, or 4 independently selected R^{1A} substituents;
each R^{1A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1A} are each optionally substituted with 1, 2, 3, or 4 independently selected R^{1B} substituents;
each R^{1B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, and OR^{a12}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{1B} are each optionally substituted with 1, 2, 3, or 4 independently selected R^{1C} substituents;
each R^{1C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, and OR^{a13};
each R^{a13} is independently selected from H, C₁₋₆ alkyl, and C₁₋₆ haloalkyl;
R² is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from H, halo, C₁₋₆ alkyl, C₁₋₆ haloalkyl, C₂₋₆ alkenyl, and C₂₋₆ alkynyl;
R⁵ is selected from H, D, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R⁵ are each optionally substituted with 1, 2, 3, or 4 independently selected R^{5A} substituents;
each R^{5A} is independently selected from halo, C₁₋₆ alkyl, C₁₋₆ haloalkyl, C₂₋₆ alkenyl, C₂₋₆ alkynyl, and CN;
R⁶ is H, halo, C₁₋₆ alkyl, C₁₋₆ haloalkyl, C₂₋₆ alkenyl, C₂₋₆ alkynyl, C₃₋₁₀ cycloalkyl-C₁₋₆ alkyl-, or (4-10 membered heterocycloalkyl)-C₁₋₆ alkyl-, wherein the C₁₋₆ alkyl, C₁₋₆ haloalkyl, C₂₋₆ alkenyl, C₂₋₆ alkynyl, C₃₋₁₀ cycloalkyl-C₁₋₆ alkyl-, and (4-10 membered heterocycloalkyl)-C₁₋₆ alkyl- are each optionally substituted by 1, 2, 3, or 4 independently selected R^{6A} substitutents;
each R^{6A} is independently selected from halo, C₁₋₆ alkyl, C₁₋₆ haloalkyl, C₂₋₆ alkenyl, C₂₋₆ alkynyl, CN, and NR^{c61}R^{d61};
each R^{c61} and R^{d61} is independently selected from H and C₁₋₆ alkyl; and
R^{c7} is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
wherein the cancer is a tumor comprising high microsatellite instability (MSI-H), mismatch repair deficient (MMRd), high tumor mutational burden (TMB-H), or mismatch repair deficient (MMRd) and high tumor mutational burden (TMB-H).

35. The method of claim 34, wherein the compound of Formula I, or a pharmaceutically acceptable salt thereof, is administered as a monotherapy.

36. The method of claim 34, wherein the compound of Formula I is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((3-chloro-4-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-((4-chloro-3-fluorophenyl)(3,3-difluorocyclobutyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof.

37. The method of claim 34, wherein the compound of Formula I is selected from:
4-((2S,5R)-4-(bis(4-fluorophenyl)methyl)-2,5-dimethylpiperazin-1-yl)-1-(((R)-tetrahydrofuran-2-yl)methyl)-1H-[1,2,3]triazolo[4,5-e][1,2,4]triazolo[4,3-a]pyrimidine;
4-((2S,5R)-4-((3,3-difluorocyclobutyl)(4-(trifluoromethyl)phenyl)methyl)-2,5-dimethylpiperazin-1-yl)-2-methyl-1-(((S)-tetrahydrofuran-2-yl)methyl)-1H-[1,2,4]triazolo[3,4-b]purine;
4-((2S,5R)-4-(bis(4-chlorophenyl)methyl)-2,5-dimethylpiperazin-1-yl)-2-methyl-1-(((S)-tetrahydrofuran-2-yl)methyl)-1H-[1,2,4]triazolo[3,4-b]purine; and
2-(4-((2S,5R)-4-(bis(4-fluorophenyl)methyl)-2,5-dimethylpiperazin-1-yl)-1H-[1,2,4]triazolo[3,4-b]purin-1-yl)-N,N-dimethylethane-1-amine;
or a pharmaceutically acceptable salt thereof.


SEQUENCE LISTING (11 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 445 aa, synthetic construct
  REGION 1..445: note = source = /note="Description of Artificial Sequence: Synthetic polypeptide"
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 218 aa, synthetic construct
  REGION 1..218: note = source = /note="Description of Artificial Sequence: Synthetic polypeptide"
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 119 aa, synthetic construct
  REGION 1..119: note = source = /note="Description of Artificial Sequence: Synthetic polypeptide"
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 5 - protein, 111 aa, synthetic construct
  REGION 1..111: note = source = /note="Description of Artificial Sequence: Synthetic polypeptide"
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 6 - protein, 5 aa, synthetic construct
  REGION 1..5: note = source = /note="Description of Artificial Sequence: Synthetic peptide"
SYWMN

SEQ ID NO: 7 - protein, 17 aa, synthetic construct
  REGION 1..17: note = source = /note="Description of Artificial Sequence: Synthetic peptide"
VIHPSDSETWLDQKFKD

SEQ ID NO: 8 - protein, 10 aa, synthetic construct
  REGION 1..10: note = source = /note="Description of Artificial Sequence: Synthetic peptide"
EHYGTSPFAY

SEQ ID NO: 9 - protein, 16 aa, synthetic construct
  REGION 1..16: note = source = /note="Description of Artificial Sequence: Synthetic peptide"
RASESVDNYGMSFMNW

SEQ ID NO: 10 - protein, 7 aa, synthetic construct
  REGION 1..7: note = source = /note="Description of Artificial Sequence: Synthetic peptide"
AASNQGS

SEQ ID NO: 11 - protein, 9 aa, synthetic construct
  REGION 1..9: note = source = /note="Description of Artificial Sequence: Synthetic peptide"
QQSKEVPYT